SEQUENCE LISTING (37 sequences)

SEQ ID NO: 1 - protein, 5 aa, Rattus norvegicus
YGGFM

SEQ ID NO: 2 - protein, 5 aa, Rattus norvegicus
YPVEP

SEQ ID NO: 3 - protein, 8 aa, Rattus norvegicus
YPVEPEEE

SEQ ID NO: 4 - protein, 10 aa, Rattus norvegicus
SPQLEDEAKE

SEQ ID NO: 5 - protein, 12 aa, Rattus norvegicus
SPQLEDEAKELQ

SEQ ID NO: 6 - protein, 11 aa, Rattus norvegicus
VGRPEWWMDYQ

SEQ ID NO: 7 - protein, 5 aa, Rattus norvegicus
YGGFL

SEQ ID NO: 8 - protein, 9 aa, Rattus norvegicus
YSKEVPEME

SEQ ID NO: 9 - protein, 26 aa, Rattus norvegicus
RKGPGPGGPGGAGGARGGAGGGPSGD

SEQ ID NO: 10 - protein, 22 aa, Rattus norvegicus
KGPGPGGPGGAGGARGGAGGGP

SEQ ID NO: 11 - protein, 25 aa, Rattus norvegicus
KGPGPGGPGGAGGARGGAGGGPSGD

SEQ ID NO: 12 - protein, 21 aa, Rattus norvegicus
GPGPGGPGGAGGARGGAGGGP

SEQ ID NO: 13 - protein, 22 aa, Rattus norvegicus
GPGPGGPGGAGGARGGAGGGPS

SEQ ID NO: 14 - protein, 24 aa, Rattus norvegicus
GPGPGGPGGAGGARGGAGGGPSGD

SEQ ID NO: 15 - protein, 22 aa, Rattus norvegicus
GPGGPGGAGGARGGAGGGPSGD

SEQ ID NO: 16 - protein, 17 aa, Rattus norvegicus
ADTGTTDEFIEAGGDIR

SEQ ID NO: 17 - protein, 16 aa, Rattus norvegicus
DTGTTDEFIEAGGDIR

SEQ ID NO: 18 - protein, 10 aa, Rattus norvegicus
EFIEAGGDIR

SEQ ID NO: 19 - protein, 9 aa, Rattus norvegicus
SPVPDLVPG

SEQ ID NO: 20 - protein, 11 aa, Rattus norvegicus
SQLQEGPPEWK

SEQ ID NO: 21 - protein, 19 aa, Rattus norvegicus
LVQTQAATDSDKVDLSIAR

SEQ ID NO: 22 - protein, 12 aa, Rattus norvegicus
TTDSDKVDLSIA

SEQ ID NO: 23 - protein, 12 aa, Rattus norvegicus
TDSDKVDLSIAR

SEQ ID NO: 24 - protein, 18 aa, Rattus norvegicus
IAQDNEPEKPVAKSETKM

SEQ ID NO: 25 - protein, 16 aa, Rattus norvegicus
QDNEPEKPVADSETKM

SEQ ID NO: 26 - protein, 15 aa, Rattus norvegicus
DNEPEKPVADSETKM

SEQ ID NO: 27 - protein, 13 aa, Rattus norvegicus
EPEKPVADSETKM

SEQ ID NO: 28 - protein, 21 aa, Rattus norvegicus
AKAPAPAAPAAEPQAEAPVAS

SEQ ID NO: 29 - protein, 30 aa, Rattus norvegicus
AKAPAPAAPAAEPQAEAPVASSEQSVAVKE

SEQ ID NO: 30 - protein, 269 aa, Rattus norvegicus
MAQFLRLCIWLLALGSCLLATVQADCSQDCAKCSYRLVRPGDINFLACTLECEGQLPSFKIWETCKDLLQVSKPEFPWDNIDMYKDSSKQDESHLLAKKYGGFMKRYGGFMKKMDELYPVEPEEEANGGEILAKRYGGFMKKDADEGDTLANSSDLLKELLGTGDNRAKDSHQQESTNNDEDSTSKRYGGFMRGLKRSPQLEDEAKELQKRYGGFMRRVGRPEWWMDYQKRYGGFLKRFAESLPSDEEGESYSKEVPEMEKRYGGFMRF

SEQ ID NO: 31 - protein, 19 aa, Rattus norvegicus
MDELYPVEPEEEANGEILA

SEQ ID NO: 32 - protein, 22 aa, Rattus norvegicus
FAESLPSDEEGESYSKEVPEME

SEQ ID NO: 33 - protein, 266 aa, Rattus norvegicus
MAQFLRLCIWLLALGSCLLATVQADCSQDCAKCSYRLVRPGDINFLACTLECEGQLPSFKIWETCKDLLQVSKPEFPWDNIDMYKDSSKQDESHLLAKKYGGFMKRYGGFMKKMDELYPVEPEEEANGEILAKRYGGFMKKDADEGDTLANSSDLLKELLGTGDNRAKDSHQQESTNNDEDSTSKRYGGFMRGLKRSPQLEDEAKELQKRYGGFMRRVGPEWWMDYQKRYGGFLKRFAESLPSDEEGESYSKEVPEMEKRYGGFMR

SEQ ID NO: 34 - protein, 99 aa, Rattus norvegicus
KKMDELYPVEPEEEANGEILAKRYGGFMKKDADEGDTLANSSDLLKELLGTGDNRAKDSHQQESTNNDEDSTSKRYGGFMRGLKRSPQLEDEAKELQKR

SEQ ID NO: 35 - protein, 16 aa, Rattus norvegicus
KRSPQLEDEAKELQKR

SEQ ID NO: 36 - protein, 66 aa, Rattus norvegicus
KRSPQLEDEAKELQKRYGGFMRRVGPEWWMDYQKRYGGFLKRFAESLPSDEEGESYSKEVPEMEKR

SEQ ID NO: 37 - protein, 33 aa, Rattus norvegicus
KRYGGFLKRFAESLPSDEEGESYSKEVPEMEKR

(19) United States
(12) Patent Application Publication
(10) Pub. No.: US 2004/0014143 A1
(43) Pub. Date: Jan. 22, 2004

(54) Method and apparatus for detecting and monitoring peptides, and peptides identified therewith

(76) Inventors: William E. Haskins, Gainesville, FL (US); Robert T. Kennedy, Ann Arbor, MI (US); David H. Powell, Gainesville, FL (US); Christopher J. Watson, Ann Arbor, MI (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/448,163

(22) Filed: May 29, 2003

Related U.S. Application Data
(60) Provisional application No. 60/384,447, filed on May 29, 2002.
(60) Provisional application No. 60/384,874, filed on May 30, 2002.

(51) Int. Cl. G01N 33/53; C07K 14/47; C07K 7/08; C07K 7/06

(52) U.S. Cl. 435/7.1; 530/324; 530/326; 530/327; 530/328; 530/329; 530/330

(57) ABSTRACT

The present invention concerns an apparatus and method for the rapid separation, detection, and characterization of molecules, such as biomolecules, within a sample. The present invention is particularly useful for the separation, detection, and characterization of peptides, such as neuropeptides, within a biological sample. The present invention also concerns neuropeptides identified using the apparatus and method of the subject invention.

### CROSS-REFERENCE TO RELATED APPLICATIONS

This application claims the benefit of provisional patent application Ser. No. 60/384,447, filed May 29, 2002, and provisional patent application Ser. No. 60/384,874, filed May 30, 2002, which are hereby incorporated by reference in their entirety, including all figures, tables, and drawings.

The subject invention was made with government support under a research project supported by the National Institutes of Health Grant No. NS38476.

### BACKGROUND OF THE INVENTION

Neuropeptides are an important and diverse class of interneuron-signaling molecules that act as hormones, neuromodulators, and neurotransmitters. Peptide signaling in the central nervous system has been implicated in many physiological and behavioral functions, including learning, memory, appetite regulation, sleep, sensory perception, immunity, and disease (Krieger, D. T. Science, 1983, 222:975-985).

Neurons produce neuropeptides by synthesizing protein precursors, which are packed into vesicles for storage and secretion. Proteases cleave precursors within the vesicles to form a collection of peptides that are released by exocytosis when the neurons are depolarized. After secretion from neurons, the peptides can be further processed by peptidases in the extracellular space. Such processing can both activate and deactivate secreted peptides. It has recently been shown that proteolytic processing produces neuropeptides that are not predicted from known protease cleavage sites (Zhang, H., et al. J of Mass Spec., 1999, 34:377-383; Fricker, L. D., et al. J. Biol. Chem., 1996, 271:30619-30624; Bures, E. J., et al. Proteomics, 2001, 1:79-92; Che, F. Y., et al. Proc. Nat. Acad. Sci. U.S., 2001, 98:9971-9976; Skold, K., et al. Proteomics, 2002, 2:447-454). In addition to neuropeptides, fragments of other proteins may be expected to be in the extracellular space and have biological significance. For example, extracellular accumulation of β-amyloid peptide, derived from the amyloid precursor protein, is observed in Alzheimer' disease (Mills, J. & Reiner, P. B. J. of Neurochem., 1999, 72:443-460).

Proteolytic processing is a complex type of post-translational modification (PTM) that generates biologically active peptides with important cell signaling properties. Tissue-specific processing (Liston, D. et al. Science, 1984, 225:734-737) of large precursor proteins is performed by proteases, endo- and exopeptidases, during intracellular transport from the rough endoplasmic reticulum where the proteins are folded by molecular chaperones to the Golgi apparatus where various PTMs are performed (Brooks, D. A. Febs Letters, 1997, 409:115-120). Further proteolytic processing occurs during stimulation and exocytotic release from a heterogenous population of secretory vesicles (SVs) into the ECF. Understanding the cascades of limited proteolytic steps by several highly specific proteases as a mechanism to increase biological diversity is important in cell signaling and in the production of recombinant proteins (Seidah, N. G. and M. Chretien Curr. Opin. Biotechn., 1997, 8:602-607).

Differential processing by various proteases produces a complex mixture of peptide intermediates and bioactive peptides. Proteases typically cleave at mono- and di-basic amino acid residues (e.g., R, K, KK and RK) flanking the N- and C-terminus of the peptide sequence within the protein precursor. The general mechanism for proteolytic processing is: 1) endopeptidase cleavage at the C-terminal side of mono- and di-basic amino acid residues, 2) carboxypeptidase cleavage of the newly exposed C-terminal mono- and di-basic amino acid residues, 3) peptidyl-amidating monooxygenase (PAM) amidation of C-terminal glycine residues and 4) aminopeptidase cleavage of one or more N-terminal residues to yield the shortened mature peptides (Rouille, Y. et al. Frontiers in Neuroendocrinology, 1995, 16:322-361).

Prediction of protease cleavage sites and proteolytic processing products (Devi, L. Febs Letters, 1991, 280:189-194) is precluded by a number of unknowns. This includes the consensus sequences and subcellular locations of the proteases involved, the kinetics of proteolysis and the tertiary and quaternary structures of the proteases and substrate. Therefore, experimental approaches are required to identify peptide products and intermediates in the various proteolytic processing pathways.

Proteolytic processing of preproenekphalin A (PEA) has been extensively studied in brain tissue (Liston, D. et al. Science, 1984; 225:734-737; Cupo, A. et al. Neuropeptides, 1984, 4:389-401; Patey, G. et al. Neuroscience, 1985, 15:1035-1044; Patey, G. et al. Life Sciences, 1983, 33:117-120; Patey, G. et al. Neuroscience Letters, 1983, 37:267-271; Fayada, C. et al. Neuropeptides, 1987, 9:9-17) and purified chromaffin granules of bovine adrenal medullas (BAM) (Wang, H. P. and Dass,C. Peptides, 2002, 23:2143-2150; Hook, V. and Metz-Boutigue,M. H. Chromaffin Cell: Trnsmitter Biosynthesis, Storage, Release, Actions, and Informatics, 2002, 971:397-405; Lembo, P. M. C. et al. Nature Neuroscience, 2002, 5:201-209; Goumon, Y. et al. Journal of Biological Chemistry, 2000, 275:38355-38362; Condamine, E. et al. Peptides, 1999, 20:865-871; Hook, V. Y. H. et al. Endocrinology, 1999, 140:3744-3754; Yasothornsrikul, S. et al. Biochemistry, 1999, 38:7421-7430; Goumon, Y. et al. Journal of Biological Chemistry, 1998, 273:29847-29856; Krieger, T. J. et al. Journal of Neurochemistry, 1992, 59:26-31; Wilson, S. P. et al. Journal of Neurochemistry, 1991, 57:870-875; Mindroiu, T. et al. Biochimica et Biophysica Acta, 1991, 1076:9-14; Ase, A. et al. Medicina-Buenos Aires, 1987, 47:606-607; Birch, N. P. and Christie, D. L. Journal of Biological Chemistry, 1986, 261:2213-2221; Watkinson, A. and Dockray, G. J. Regulatory Peptides, 1987, 18:350; Dumont, M. and Lemaire, S. Federation Proceedings, 1987, 46:1448; Birch, N. P. and Christie, D. L. Journal of Biological Chemistry, 1986, 261:2213-2221). These studies revealed that PEA-processing occurs via successive proteolytic events by various proteases such as preprotein convertase 1/3 and 2 (PC1/3 and PC2) to generate biologically active peptides such as the neuropeptides Met- and Leu-enkephalin. However, a problem with these studies is that no discrimination was made between intra- and extracellular-peptides.

More recently, in vivo microdialysis has enabled PEA processing to be studied in the extracellular fluid (ECF) (Shen, H. et al. J. Chromatography B., 1997, 704:43-52; Zhang, H. et al.i J. Mass Spectro., 1999, 34:377-383; Emmett, M. R. et al. Journal of Neuroscience Methods, 1995, 62:141-147; Bergasa, N. V. et al. Life Sciences, 1997, 61:1169-1175; Haskins, W. E. et al. Analytical Chemistry, 2001, 73:5005-5014; Lapeyre, S. et al. Naunyn-Schmiedebergs Archives of Pharmacology, 2001, 363:399-406). In vivo microdialysis enables dynamic measurements of the ECF in discrete regions with minimal damage to the surrounding tissue (Ungerstedt, U. and Hallstrom, A. Life Sciences, 1987, 41:861-864). The microdialysis probe mimics the action of a capillary blood vessel by passively sampling the ECF for molecules involved in cell signaling. Alternatively, it can be used to administer compounds in order to determine pharmacological effects.

One route to study processing of neuropeptides is by analysis of brain tissue. Although direct measurement of extracellular peptide levels in vivo is invaluable in elucidating peptide function, more indirect methods such as the effect of pharmacological agents or the postmortem analysis of tissue for peptides or mRNA are typically used in studies of neuropeptides. These indirect methods are popular because sampling techniques for in vivo monitoring, such as microdialysis, produce microliter samples with attomole quantities of peptide. However, such small sample volumes make analytical measurements difficult.

When in vivo peptide monitoring is performed, a popular analytical technique is radioimmunoassay (RIA) because it has excellent specificity and, with appropriate optimization, can achieve detection limits of 100 attomoles (amol) in a microliter fraction (Maidment, N. T. et al., J. Neuroscience, 1989, 33:549-557). Despite the high specificity, RIA results can be ambiguous due to cross-reactivity of the labeled antibody with similar peptides (Lovelace, J. L. et al., J. Chromatogr., 1991, 562:573-584; Nilsson, C. L. et al., Peptides, 1998, 19:781-789). When RIA is combined with high performance liquid chromatography (HPLC), specificity limitations can be minimized; however, this combination is a cumbersome procedure that is usually avoided. Furthermore, RIAs are usually limited to measurement of only a single peptide per animal. Thus, relationships between different peptides and stimuli must be inferred from data collected from different animals. Finally, RIA generally does not allow monitoring or discovery of novel neuropeptides. In the past, discovery of novel neuropeptides was accomplished by preparative-scale isolation of bioactive components, purification, and Edman sequencing (Strand, F. L. Neuropeptides: Regulators of Physiological Processes, MIT Press: Cambridge, Mass. 1999).

Newer approaches to detection of endogenous neuropeptides in dialysate, including capillary liquid chromatography (CLC) with electrochemical detection (CLC-EC) (Shen, H. et al., J. Chromatogr., 1997, 704:43-52; Shen, H. et al., Anal. Chem., 1999, 71:987-994) and tandem mass spectrometry (MS²) (Emmett, M. R. et al., J. Neurosci. Methods, 1995, 62:141-147; Andren, P. E. et al., Brain Res., 1999, 845:123-129), have been reported recently. MS² is attractive because it offers the possibility of detecting peptides with sequence specificity and can be used, in principle, for any peptide. In the seminal work on MS² for in vivo peptide detection, 10-μL microdialysis samples were preconcentrated and desalted on 50-μm i.d. columns coupled to a triple-quadrupole mass spectrometer by a microelectrospray interface (Emmett, M. R. et al., J. Neurosci. Methods, 1995, 62:141-147; Andren, P. E. et al., Brain Res., 1999, 845:123-129). Endogenous Met-enkephalin (Emmett, M. R. et al., J. Neurosci. Methods, 1995, 62:141-147) and neurotensin (Andren, P. E. et al., Brain Res., 1999, 845:123-129) have been measured in microdialysate fractions using the selected reaction monitoring (SRM) scan mode, where only one or a few precursor-to-product ion transitions are monitored over a narrow scan range (e.g., 2 m/z). This method was revolutionary in its use, but step gradients and SRM precluded multianalyte monitoring or characterization of unknown peptides.

The advent of proteomics has greatly expanded the power of brain tissue analysis. Using proteomics techniques, peptides formed by tissue proteases are isolated from the sample and then analyzed by CLC-MS². The resulting MS² spectra can be correlated to protein precursors by database searching. This method is similar to the “shot-gun” proteomics method in which a protein mixture is pretreated by selective proteolysis (e.g., trypsin digestion) to form a collection of peptides characteristic of the proteins and the protease used for proteolysis prior to CLC-MS² analysis and database searching. Application of this method to peptides in tissue is complicated by the lack of control over the proteases used for digestion, i.e., not all of the peptides are tryptic peptides. This creates at least two difficulties. Tryptic peptides have basic sites resulting in good ionization efficiencies and efficient cleavage along the peptide backbone promoting a high yield of b- and y-type ions (Yates, J. R., J. Mass Spect., 1998, 33:1-19). Peptides produced by other proteases may be less sensitively detected. In addition, without a priori knowledge of the protease specificity, a greater library of peptides must be searched, thus increasing the probability of finding a random match. Despite these limitations, many new peptides have been found by this approach (Bures, E. J., et al. Proteomics, 2001, 1:79-92; Che, F. Y., et al. Proc. Nat. Acad. Sci. U.S., 2001, 98:9971-9976; Skold, K., et al. Proteomics, 2002, 2:447-454).

A problem with tissue analysis for studying neuropeptides is that no discrimination is made between intracellular peptides and those that are actually released into the extracellular space. In addition, tissue analysis could not be used for correlating behavior to a given set of released peptides. Analysis of peptides in the extracellular space of live animals is required for such studies. Recently, peptides in the extracellular compartment of live rats have been detected by microdialysis sampling coupled with CLC-MS² or CE-MS; however, these studies have relied on detecting just a few known peptides or infusing a known peptide at high levels to follow peptide processing in vivo. Such approaches preclude identification of novel endogenous species.

It would be advantageous to have available a method and apparatus that provide full-scan MS² at a sensitivity comparable to SRM experiments on triple-quadrupole instruments (Tiller, P. R. et al., Rapid Commun. Mass Spectrom., 1997, 11:1151-1153). In addition to improving confidence in the measurement of known peptides (Lily, Y. T. et al., Anal. Chem., 1996, 68:3397-3404), full-scan MS² offers the possibility of characterizing novel peptides by sequencing at attomole levels (Hunt, D. F. et al., Science, 1992, 255:1261-1263; Henderson, R. A. et al., Proc. Natl. Acad. Sci. U.S.A., 1993, 90:10275-10279; den Haan, J. M. et al., Science, 1998, 279:1054). For example, it would be advantageous to have available a method and apparatus for exploring endogenous peptide content and the proteome of brain extracellular fluid in living humans and animals that would accommodate extremely low concentrations of endogenous peptides within a sample (e.g., 1-100 pM) and small sample volumes (1-100 μL). Development of such a method and apparatus would allow many novel studies such as identification of processing patterns after release, correlation of peptides released into the extracellular fluid with different behavioral and physiological states, and identification of potentially novel neurotransmitters.

### BRIEF DESCRIPTION OF THE INVENTION

The present invention concerns an apparatus and method for the rapid separation, detection, and characterization of molecules, such as biomolecules, within a sample. The present invention is particularly useful for the separation, detection, and characterization of peptides, such as neuropeptides, within a biological sample. Detection of multiple known peptides at low-attomole levels (low picomolar concentrations) in complex samples has been achieved using the apparatus and method of the present invention.

The apparatus and method of the subject invention have been demonstrated to operate at a high flow rate of 370 nL/min. during sample loading and a low flow rate of 10 nL/min. during chromatographic separation. Advantageously, the high flow rate during sample loading minimizes sample preconcentration and desalting time. In addition, the low flow rate during separation results in improved sensitivity due to the increase in separation, ionization, and ion transfer efficiency.

In one aspect, the invention pertains to an apparatus for collecting and analyzing a sample, such as a biological sample. The apparatus is a multi-pump system that allows high-flow rates for sample precencentration and desalting and low flow rates for separation and electrospray. The apparatus of the subject invention includes a means for collecting and delivering a sample to means for chromatographic separation, such as a chromatographic separation column, which is in fluid communication with the sampling means. The collecting means is preferably a microdialysis probe. Preferably, the chromatographic separation means is in operable communication with a means for detecting the sample separated by the chromatographic separation means, such as a mass spectrometer. The chromatographic separation column is preferably a liquid chromatography column having an integrated electrospray emitter. The detection means is preferably a spectrometer. More preferably, the mass spectrometer is a quadrupole ion trap mass spectrometer. The integrated electrospray emitter is interfaced with the mass spectrometer.

In another aspect, the subject invention concerns an integrated separation column-electrospray emitter including a capillary tube with an inlet end and an outlet end. The outlet end of the capillary tube integrally forms an electrospray emitter. The inner diameter and outer diameter of the integrated electrospray emitter taper and terminate in a spray orifice. Preferably, the spray orifice of the electrospray emitter has an inner diameter within the range of about 2 μm to about 5 μm. More preferably, the spray orifice of the electrospray emitter has an inner diameter of about 3 μm. The capillary tube defines a separation channel and contained within the separation channel is a frit. The frit is positioned upstream from the orifice of the integrated electrospray emitter and is preferably positioned about 3 cm or less from the emitter spray orifice. More preferably, the frit is positioned about 1 cm upstream from the emitter spray orifice.

The frit is preferably a macroporous frit. More preferably, the frit is a macroporous polymer frit formed by in situ photopolymerization. The integrated separation column-emitter can further include a separation medium capable of separating a sample into its components when the sample is passed through the separation medium within the capillary tube. The capillary tube has an internal diameter from about 20 μm to about 30 Mm, and is defined by a capillary tube wall. Preferably, the internal diameter of the capillary tube is about 25 μm.

In another aspect, the subject invention concerns a method for identifying and monitoring peptides in vivo by collecting and analyzing a sample using the apparatus of the present invention. Detection of multiple known peptides at low-attomole levels (low-picomolar concentrations) in complex samples was achieved by using the apparatus and method of the subject invention. The invention allows online and offline monitoring of endogenous neuropeptides at basal and stimulated levels with at least 30-min. temporal resolution in microdialysis samples. Unknown peptides can be sequenced at attomole levels, using time-segmented and data-dependent MS² and MS³ scans, and optionally correlated to neuropeptide precursors using database searching. Validation of peptide sequences was achieved by comparison of retention time, MS² spectra, and MS³ spectra with that of a synthetic peptide and by de novo sequence interpretation. The method of the subject invention is particularly useful for monitoring known peptides, studying processing of endogenous peptides, characterizing novel peptides that are potentially neuroactive, and identifying the localized neurochemical changes and biomarkers that occur in response to any physiological state.

In a further aspect, the present invention relates to peptides identified using the method and apparatus of the subject invention, and nucleic acids encoding such peptides. These peptides represent cleavage products of proenkephalin A (PEA), neurogranin, fibrinogen alpha chain precursor, fibrinogen beta chain precursor, excitatory amino acid transporter 1, and brain acidic membrane protein. The peptides of the subject invention include YGGFM (SEQ ID NO: 1) (Met-enkephalin), YPVEP (SEQ ID NO: 2), YPVEPEEE (SEQ ID NO: 3), SPQLEDEAKE (SEQ ID NO: 4), SPQLEDEAKELQ (SEQ ID NO: 5), VGRPEWWMDYQ (SEQ ID NO: 6), YGGFL (SEQ ID NO: 7) (Leu-enkephalin), YSKEVPEME (SEQ ID NO: 8), RKGPGPGGPGGAGGARGGAGGGPSGD (SEQ ID NO: 9), KGPGPGGPGGAGGARGGAGGGP (SEQ ID NO: 10), KGPGPGGPGGAGGARGGAGGGPSGD (SEQ ID NO: 11), GPGPGGPGGAGGARGGAGGGP (SEQ ID NO: 12), GPGPGGPGGAGGARGGAGGGPS (SEQ ID NO: 13), GPGPGGPGGAGGARGGAGGGPSGD (SEQ ID NO: 14), GPGGPGGAGGARGGAGGGPSGD (SEQ ID NO: 15), ADTGTTDEFIEAGGDIR (SEQ ID NO: 16), DTGTTDEFIEAGGDIR (SEQ ID NO: 17), EFIEAGGDIR (SEQ ID NO: 18), SPVPDLVPG (SEQ ID NO: 19), SQLQEGPPEWK (SEQ ID NO: 20), LVQTQAATDSDKVDLSIAR (SEQ ID NO: 21), TTDSDKVDLSIA (SEQ ID NO: 22) (Fibrinopeptide B₁₋₁₃), TDSDKVDLSIAR (SEQ ID NO: 23) (Fibrinopeptide B₂₋₁₄), IAQDNEPEKPVAKSETKM (SEQ ID NO: 24), QDNEPEKPVADSETKM (SEQ ID NO: 25), DNEPEKPVADSETKM (SEQ ID NO: 26), EPEKPVADSETKM (SEQ ID NO: 27), AKAPAPAAPAAEPQAEAPVAS (SEQ ID NO: 28), AKAPAPAAPAAEPQAEAPVASSEQSVAVKE (SEQ ID NO: 29), MDELYPVEPEEEANGEILA (SEQ ID NO: 31), FAESLPSDEEGESYSKEVPEME (SEQ ID NO: 32), MAQFLRLCIWLLALGSCLLATVQADCSQDCAKCSYRLVRPGDINFLACTLECEGQLP SFKIWETCKDLLQVSKPEFPWDNIDMYKDSSKQDESHLLAKKYGGFMKRYGGFMK KMDELYPVEPEEEANGEILAKRYGGFMKKDADEGDTLANSSDLLKELLGTGDNRA KD SHQQESTNND EDSTSKRYGGFMRGL KRSPQLEDEAKELQKR YGGFMRRVGPEWWMDYQKRYGGFLKRFAESLPSDEEGESYSKEVPEME KR YGGFMR (SEQ ID NO: 33), KKMDELYPVEPEEEANGEILAKRYGGFMKKDADEGDTLANS SDLLKELLGTGDNR AKD SHQQESTNND EDSTSKRYGGFMRGLKRSPQLEDEAKELQKR (SEQ ID NO: 34), KRSPQLEDEAKELQKR (SEQ ID NO: 35), KRSPQLEDEAKELQKRYGGFMRRVGPEWWMDYQKRYGGFLKRFAESLPSDEEGES YSKEVPEMEKR (SEQ ID NO: 36), and KRYGGFLKRFAESLPSDEEGESYSKEVPEMEKR (SEQ ID NO: 37), and fragments and variants of any of the foregoing.

Met-enkephalin and Leu-enkephalin represent known cleavage products of PEA; however, the other peptides represent novel cleavage products of the above protein precursors. Specifically, peptides of SEQ ID NOs: 2-6 and 8 are cleavage products of PEA. Peptides of SEQ ID NOs: 9-15 are cleavage products of neurogranin. Peptides of SEQ ID NOs: 16-20 are cleavage products of fibrinogen alpha chain precursor. Peptides of SEQ ID NOs: 21-23 are cleavage products of fibrinogen beta chain precursor. Peptides of SEQ ID NOs: 24-27 are cleavage products of excitatory amino acid transporter 1. Peptides of SEQ ID NOs: 28 and 29 are cleavage products of brain acidic membrane protein.

The subject invention also concerns methods for modulating the level and/or activity of neurotransmitters within the brain. In particular, the subject invention concerns methods for increasing the endogenous levels of gamma-aminobutyric acid (GABA) and/or aspartate in vivo or in vitro by administration of a peptide of the present invention to a subject. In one embodiment, PEA or a biologically active fragment or variant of PEA is administered to a subject. Preferably, a cleavage product of PEA is administered to the subject, such as those of SEQ ID NOs. 1-8. Alternatively, a nucleotide sequence encoding a peptide of the subject invention can be administered to a subject and expressed.

### BRIEF DESCRIPTION OF THE DRAWINGS

FIGS. 1A-1E show block diagrams of an embodiment of the automated two-pressure CLC-MS² system of the subject invention. Valve 1 is used for pump selection and valve 2 is the injection valve. S1 and S2 are splitter capillaries (1 m×50 μm i.d.) that can be shut off with valves (hexagons) as shown. W is a waste port. HV is the stainless steel union where high voltage is applied to generate electrospray. The apparatus set up for sample loading, preconcentration, desalting, separation/electrospray, and tuning/electrospray is shown in FIG. 1A-1E, respectively.

FIGS. 2A-2D show CLC columns with integrated electrospray emitters. FIG. 2A shows a scanning electron micrograph of the column upstream of the frit. FIG. 2B shows a scanning electron micrograph of a macroporous flit formed by in situ photopolymerization (scale bar 10 μm). After the frit was prepared, the capillary was cleaved to expose the inner column. FIG. 2C shows a bright-field optical image of the end of the LC column with an emitter tip. FIG. 2D shows a SEM of an electrospray emitter with end-on view (scale bar 5 μm).

FIG. 3 shows the effect of the gradient steepness parameter on resolution (R) and sensitivity (peak height) as a function of flow rate. Dashed line is 20 nL/min, and solid line is 70 nL/min. R given by ▪ for 20 nL/min. and ♦ for 70 nL/min. Peak height is given by ● for 20 nL/min. and ▴ for 70 nL/min. Resolution was calculated for 370-nL injections of 18 nM Met- and Leu-enkephalin (6.6 fmol injected on-column). Peak height was determined for Leu-enkephalin. Data are averages from three columns (all 2 cm long), and error bars represent 1 standard deviation. The gradient was from 5 to 90% B with 0.1≦b≦100 and 0.1<t_{G}≦12.5 minutes.

FIG. 4 shows the effect of the gradient steepness parameter (b) on resolution (R) for different column lengths and flow rates (▪, 2-cm-long column at 20 nL/min.; ●, 10-cm-long column at 50 nL/min.; ♦, 2-cm-long column at 70 nL/min.). Chromatographic conditions are the same as described for FIG. 3. Data are the average from three columns and error bars are 1 standard deviation.

FIGS. 5A-5C show high-sensitivity CLC-MS² measurement of a mixture of Met-enkephalin and Leu-enkephalin at 33 pM (1.8 μL injected corresponding to 59 amol of each peptide loaded onto the column) each in aCSF using a 2-cm column. FIG. 5A shows time-segmented total ion chromatogram, showing peaks for Met-enkephalin (7.8 min) and Leu-enkephalin (8.5 min). FIG. 5B shows a reconstructed ion chromatogram for Leu-enkephalin, monitoring the 556→397+425 m/z transition. FIG. 5C shows full-scan MS² for Leu-enkephalin obtained from the chromatographic peak showing all of the expected b- and and y-type ions. The gradient was from 5 to 90% B with b=2.0, t_{G}=5 min, and flow rate 20 nL/min.

FIGS. 6A-6D show an illustration of peptide carry-over in the injection valve and carry-over prevention, using deuterated standards. Reconstructed ion chromatograms from injection of 1.8 μL of 600 pM YGGF_{D5}L (FIG. 6A) followed by injection of 1.8 μL of 60 pM YGGFL (FIG. 6B). In FIGS. 6A and 6B, the upper trace is current for the 556→397+425 m/z transition of YGGFL and the lower trace is for the 561→402+430 m/z transition of YGGF_{D5}L. FIG. 6C shows MS² from the peak of the chromatogram shown in FIG. 6A, illustrating the a₄ and b₄ ions of YGGF_{D5}L (402 and 430 m/z). FIG. 6D shows MS² from the peak of the chromatogram in FIG. 6B, illustrating resolution of a₄ and b₄ ions for YGGF_{D5}L and YGGFL (397 and 425 m/z).

FIGS. 7A-7F shows in vivo detection of Met- and Leu-enkephalin during basal and K⁺-stimulated conditions. FIG. 7A shows a total ion chromatogram (top panel) and reconstructed ion chromatogram (bottom panel) for a globus pallidus dialysate sample (2.0 μL) collected during basal conditions. The reconstructed ion chromatogram is time-segmented and shows the current for the 574→397+425 (8.0-9.6 min) and 556→397+425 (9.6-11.0 min) m/z transitions of Met- and Leu-enkephalin, respectively. Met-enkephalin elutes at 9.3 min and Leu-enkephalin at 9.9 min. FIGS. 7C and 7E show the mass spectra obtained at the elution time of Met-enkephalin and Leu-enkephalin, respectively, for the chromatogram shown in FIG. 7A. FIGS. 7D and 7F show the mass spectra obtained at the elution time of Met-enkephalin and Leu-enkephalin, respectively, for the chromatogram shown in 7B.

FIG. 8 shows in vivo monitoring results for Met-enkephalin (▪), leu-enkephalin(●), and an unknown peptide (▴) for a single rat. Each data point is from a 2-μL sample collected at the time indicated. The bar indicates application of 150 mM K⁺ to the microdialysis probe.

FIGS. 9A-9D show in vivo identification of peptides by data-dependent LC-MS². FIG. 9A shows a total ion chromatogram for data-dependent MS² of a dialysate sample collected from the globus pallidus during K⁺ stimulation. FIGS. 9B, 9C, and 9D show MS² of SPQLEDEAKE (SEQ ID NO: 4), Met-enkephalin (SEQ ID NO: 1), and Leu-enkephalin (SEQ ID NO: 7), respectively.

FIGS. 10A-10C show sequencing of a novel peptide by time-segmented MS² from sample collected in vivo during K⁺-induced depolarization. FIG. 10A a total ion chromatogram (top panel) and reconstructed ion chromatogram (bottom panel) for the 574→472+529 m/z transition. FIGS. 10B and 10C show MS² of SPQLEDEAKE (SEQ ID NO: 4) obtained in vivo and from synthetic peptide, respectively. Synthetic peptide was injected at 600 pM.

FIGS. 11A-11C shows confirmation of the sequence of a novel peptide by time-segmented MS³ from sample collected in vivo during K⁺-induced depolarization. FIG. 11A shows a total ion chromatogram for the 574→833→ transition. FIGS. 11B and 11C show MS³ of the SPQLEDEAKE (SEQ ID NO: 4) peptide obtained in vivo and from synthetic peptide (600 pM), respectively.

FIG. 12 shows the amino acid sequence of preproenkephalin A (SEQ ID NO: 30). Sequences matching Met-enkephalin (SEQ ID NO: 1) and Leu-enkephalin (SEQ ID NO: 7) are underlined; however, these peptides are not necessarily released by preproenkephalin A processing. Peptide I, which has been observed in previous work on tissues (Stern, A. S. et al., Proc. Natl. Acad. Sci. U.S.A., 1981, 78:1962-1966), is in boldface type. A peptide identified using the apparatus and method of the subject invention (referred to herein as peptide I₁₋₁₀) is shown in the box (SPQLEDEAKE (SEQ ID NO: 4)).

FIG. 13 shows a scheme for monitoring and discovering neuropeptides using the apparatus and method of the subject invention.

FIGS. 14A-14I show triplicate, 0.4 μL, injections of a tryptic digest of lysozyme, carbonic anhydrase, and conalbumin at 10 nM with 1 nM YGGFD₅L (400 amol injected on-column). Base peak reconstructed ion chromatograms (RIC) show the most abundant precursor ions observed in vitro (FIGS. 14A-14C) and the product ions for the YGGF_{D5}L precursor ion at 561 m/z (FIGS. 14D-14F). Data-dependent MS² spectra of the precursor ion at 561 m/z in (FIGS. 14G-14I) show the characteristic a₄ and b₄ product ions (402 m/z and 430 m/z, respectively) of YGGF_{D5}L. Peaks are labeled by the most abundant product ion of the data-dependent MS² spectrum (e.g., 402 m/z for YGGF_{D5}L).

FIGS. 15A-15D show in vivo microdialysis-CLC-MS² analysis of CSF collected from the rat striatum during K⁺-induced release conditions. FIG. 15A shows a total ion chromatogram. FIG. 15B shows the base peak RIC for the most abundant product ion and (FIG. 15C) RIC for the 556→397+425 transition of YGGFL. Peaks are labeled by the most abundant product ion of the data-dependent MS² spectrum (FIGS. 15B and 15C) (e.g., 397 m/z for YGGFL (SEQ ID NO: 7)). FIG. 15D shows data-dependent MS² spectrum of the precursor ion at 556 m/z. The characteristic a₄ and b₄ product ions (397 m/z and 425 m/z, respectively) of YGGFL (SEQ ID NO: 7) (FIG. 15B).

FIGS. 16A-16D show a comparison of total ion chromatograms (TICs) and reconstructed ion chromatograms (RICs) for data-dependent MS² of dialysate collected from rat striatum during basal (FIGS. 16B and 16D, respectively) and depolarizing (FIGS. 16A and 16C, respectively) conditions showing the most abundant precursor ions observed in vivo. Peaks are labeled by retention time in total ion chromatograms (FIGS. 16A and 16B) and by the most abundant product ion of the MS² spectrum in reconstructed ion chromatograms (FIGS. 16C and 16D).

FIGS. 17A-17F show representative MS² spectra and sequences for peptides produced from proteolytic processing of PEA (FIG. 17A), neurogranin (FIG. 17B), fibrinogen α (FIG. 17C), fibrinogen β (FIG. 17D), excitatory amino acid transporter (FIG. 17E), and brain acidic membrane protein (FIG. 17F). The number assigned to the peptide in Table 2 is listed below the sequence for clarity.

FIGS. 18A-18C show MS² spectra (FIGS. 18A and 18C) from the in vivo library and the difference MS² spectrum (FIG. 18B) for SPQLEDEAKE (SEQ ID NO: 4) observed in two rats. Database-searching programs incorrectly assigned the sequence AIKNGWLSEE to the MS² spectrum in (C).

FIG. 19 shows a summary of the in vivo results illustrating the six-step data-reduction strategy described in the experimental section. The number of MS² spectra remaining and the % of the total number of MS² spectra remaining are plotted vs. the data-reduction step (see experimental section).

FIGS. 20A-20H show MS² spectra and sequences for peptides produced from proteolytic processing of PEA.

FIGS. 21A-21G show MS² spectra and sequences for peptides produced from proteolytic processing of neurogranin.

FIGS. 22A-22H show MS² spectra and sequences for peptides produced from proteolytic processing of fibrinogen α (FIGS. 22A-22D) and fibrinogen β (FIGS. 22E-22H).

FIGS. 23A-23F show MS² spectra and sequences for peptides produced from proteolytic processing of excitatory amino acid transporter (FIGS. 23A-23D) and brain acidic membrane protein (FIGS. 23E-23F). The number assigned to the peptide in Table 3 is listed below the sequence for clarity.

FIGS. 24A-24F show in vivo microdialysis-CLC-MS² analysis of CSF collected from the rat striatum during depolarization-induced release of peptides into the ECF. FIGS. 24A-24C illustrate the separation of peptides and product ions during CLC-MS² analysis of dialysate. FIG. 24A shows a MS² contour plot of the most abundant product ions observed during a data-dependent MS² experiment. FIG. 24B shows the total ion chromatogram (TIC) and FIG. 24C shows the base peak reconstructed ion chromatogram (RIC) for the most abundant product ions. An arrow indicates the peak corresponding to YGGFL (SEQ ID NO: 7). FIGS. 24D-24F illustrate how data-dependent MS² spectra were collected in the ‘triple play’ scan mode (MS, zoom, MS²). FIG. 24D shows a MS scan where precursor ion for YGGFL (SEQ ID NO: 7) at 556 m/z exceeded the threshold for data-dependent MS² analysis. FIG. 24E shows a zoom scan for charge state determination of the 556 m/z precursor ion in (FIG. 24D). A Δm/z=1.0 between the ¹²C and ¹³C isotopes yields z=+1 and a [M+H]⁺ precursor ion. FIG. 24F shows the data-dependent MS² spectrum of the singly charged precursor ion at 556 m/z in (FIG. 24D) with the characteristic a₄ and b₄ product ions (397 m/z and 425 m/z, respectively) of YGGFL (SEQ ID NO: 7).

FIG. 25 shows a non-exhaustive depiction of selected PEA-derived peptides by amino acid position. Unshaded peptides contain the sequence YGGFX, where X is M or L, while shaded peptides do not. The peptide length in amino acids is also shown. Hypothetical intermediates (HIs) with mono- or di-basic sites (listed by animal number) and without (SPQLEDEAKELQ (SEQ ID NO: 5), MDELYPVEPEEEANGEILA and FAESLPSDEEGESYSKEVPEME) are shown for the 7 of 13 animals where PEA processing was observed. Peptides observed in this work are indicated by an arrow and HIs are indicated by a dashed arrow.

FIG. 26 shows hypothetical intermediates (HIs) of PEA processing and novel PEA-processing patterns observed in vivo. HIs with and without (underlined) mono- or di-basic sites for all of the PEA-derived peptides observed in this work are shown for the 7 of 13 animals where PEA processing was observed (i.e., animals 1,2,4 and 10-13 in Table 2). Predicted mono- and di-basic cleavage sites (i.e., K, R, KR, KK and RR), peptides observed (numbered by amino acid position according to Table 6) and selected other amino acids comprising the hypothetical intermediate sequences (e.g., Q for peptides 4-7 (SEQ ID NOs: 4-7)) are shown. His are relatively large peptides containing all of the peptide sequences observed in each animal (not to scale).

FIGS. 27A-27B shows in vivo effect of peptide I₁₋₁₀ (SEQ ID NO: 4) on neurotransmitter levels. In vivo microdialysis-capillary electrophoresis with laser-induced fluorescence detection revealed a 3-4 fold increase in aspartate (Asp) and gamma-amino butyric acid (GABA) levels during perfusion of 10 μM peptide I₁₋₁₀ through the microdialysis probe. An example electropherogram is shown in (FIG. 27A) and the temporal response of Asp, GABA, and Glu are shown in (FIG. 27B).

FIGS. 28A-28B show that in vivo microdialysis-CLC-MS² base peak reconstructed ion chromatograms (RICs) show the most abundant product ions observed during sleeping (FIG. 28A) and prolonged-wakefulness (FIG. 28B) states. The MS² spectrum for the doubly charged unknown precursor ion at 1282 m/z and its most abundant product ion at 1373 m/z is labeled with an asterisk (FIG. 28B). The MS² spectrum for the unknown precursor ion at 1282 m/z was observed only in the forced-wakefulness state (FIG. 28B). The microdialysis probe was implanted in the hypothalamus of a male rat.

### BRIEF DESCRIPTION OF SEQUENCES

SEQ ID NO: 1 is Met-enkephalin, a peptide of preproenkephalin A (PEA).

SEQ ID NOs: 2-6 are peptides and cleavage products of PEA.

SEQ ID NO: 7 is Leu-enkephalin, a peptide of PEA.

SEQ ID NO: 8 is a peptide and cleavage product of PEA.

SEQ ID NOs: 9-15 are peptides and cleavage products of neurogranin.

SEQ ID NOs: 16-20 are peptides and cleavage products fibrinogen alpha chain precursor.

SEQ ID NOs: 21-23 are peptides and cleavage products of fibrinogen beta chain precursor.

SEQ ID NOs: 24-27 are peptides and cleavage products of excitatory amino acid transporter 1.

SEQ ID NOs: 28-29 are peptides and cleavage products of brain acidic membrane protein.

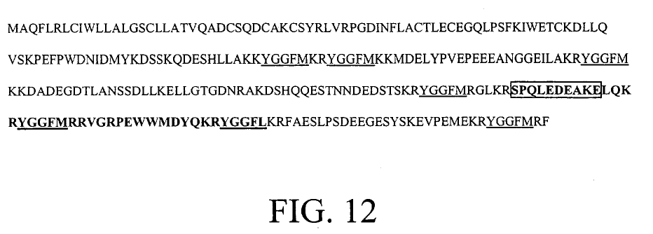

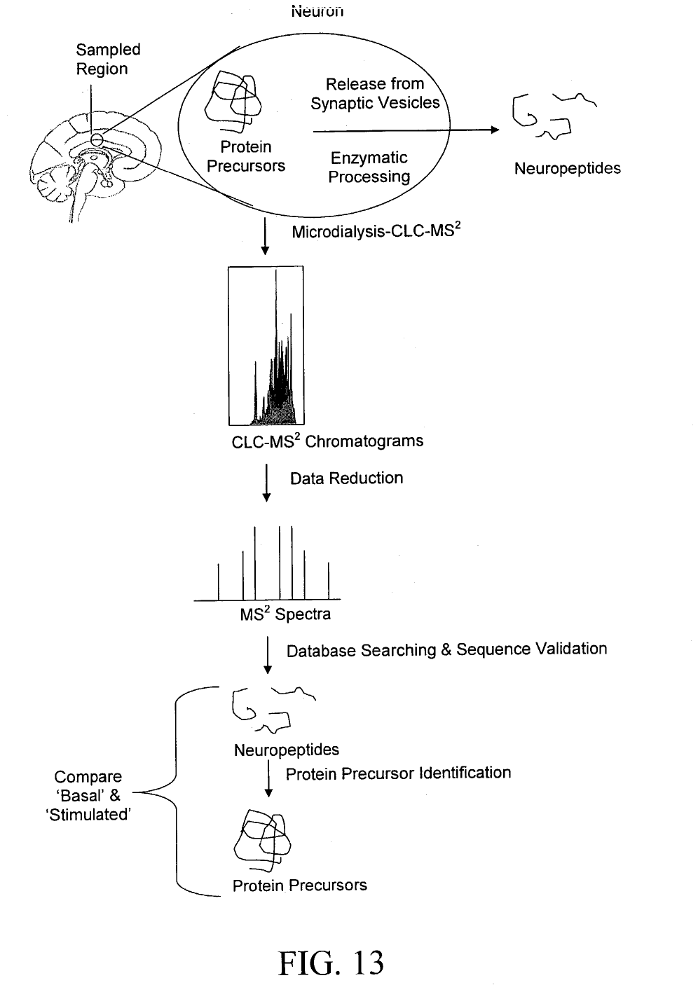

SEQ ID NO: 30 is the amino acid sequence of PEA, shown in FIG. 12.

SEQ ID NOs: 31-37 are hypothetical intermediates, shown in FIG. 26.

### DETAILED DISCLOSURE OF THE INVENTION

The subject invention concerns a new and efficient apparatus for the rapid separation, detection, and characterization of molecules, such as biomolecules, within a sample. The apparatus of the present invention is particularly useful for the separation, detection, and characterization of peptides, such neuropeptides, within a biological sample. Detection of multiple known peptides at low-attomole levels (low picomolar concentrations) in complex samples has been achieved using the apparatus and method of the present invention.

The apparatus of the subject invention has been demonstrated to operate at a high flow rate of 370 nL/min. during sample loading and a low flow rate of 10 nL/min. during chromatographic separation. The high flow rate during sample loading minimizes sample preconcentration and desalting time. The low flow rate during separation results in improved sensitivity due to the increase in separation, ionization, and ion transfer efficiency.

In one aspect, the invention pertains to an apparatus for collecting and analyzing a sample, such as a biological sample. Advantageously, the apparatus is a multi-pump system that allows high-flow rates for sample loading and low flow rates for elution. The apparatus of the subject invention includes a means for collecting and delivering a sample to a means for chromatographic separation, such as a chromatographic separation column, which is in fluid communication with the collecting means. The collecting means is preferably a microdialysis probe. The chromatographic separation means is in operable communication with a means for detecting the sample separated by the chromatographic separation means, such as a mass spectrometer. The chromatographic separation means is preferably a liquid chromatography column having an integrated electrospray emitter. The detection means is preferably a spectrometer. More preferably, the mass spectrometer is a quadrupole ion trap mass spectrometer. The integrated electrospray emitter is interfaced with the mass spectrometer.

In another aspect, the subject invention concerns an integrated separation column-electrospray emitter including a capillary tube with an inlet end and an outlet end. The outlet end of the capillary tube integrally forms an electrospray emitter. The inner diameter and outer diameter of the integrated electrospray emitter taper and terminate in a spray orifice. Preferably, the spray orifice of the electrospray emitter has an inner diameter within the range of about 2 μm to about 5 μm. More preferably, the spray orifice of the electrospray emitter has an inner diameter of about 3 μm. The capillary tube defines a separation channel and contained within the separation channel is a frit. The frit is positioned upstream from the orifice of the integrated electrospray emitter and is preferably positioned about 3 cm or less from the emitter spray orifice. More preferably, the frit is positioned about 1 cm upstream from the emitter spray orifice.

The frit is preferably a macroporous frit. More preferably, the frit is a macroporous polymer frit formed by in situ photopolymerization. Most preferably, the frit is a macroporous frit formed by in situ photopolymerization of glycidyl methacrylate and trimethylolpropane trimethacryalte. The integrated separation column-emitter can further include a separation medium capable of separating a sample into its components when the sample is passed through the separation medium within the capillary tube. The capillary tube has an internal diameter from about 20 μm to about 30 μm, and is defined by a capillary tube wall. Preferably, the internal diameter of the capillary tube is about 20 μm.

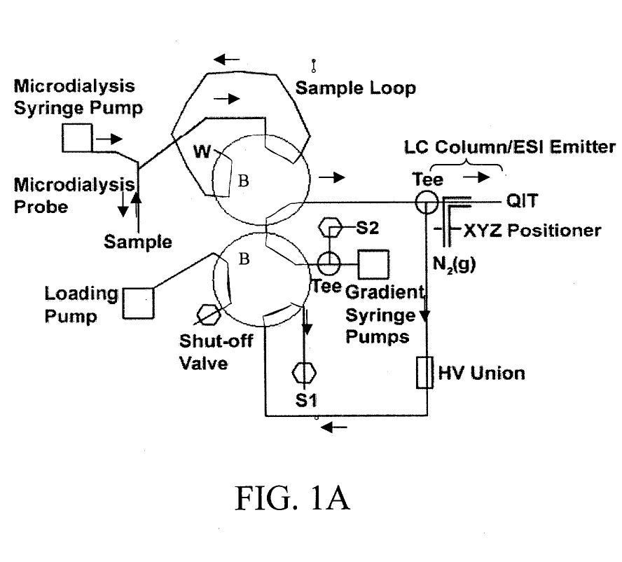

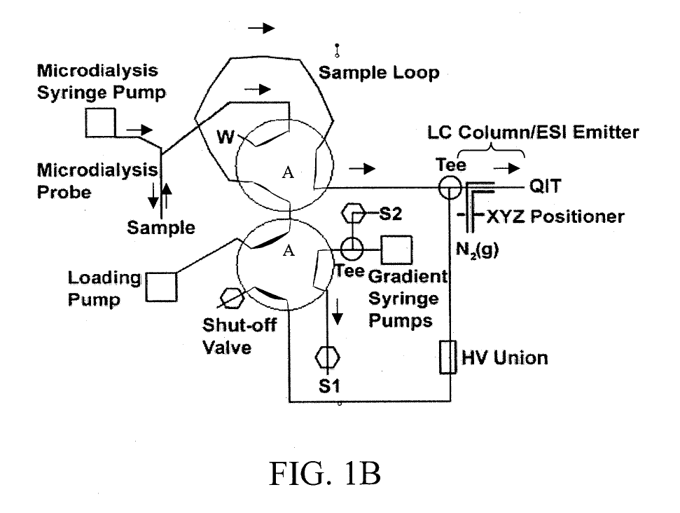

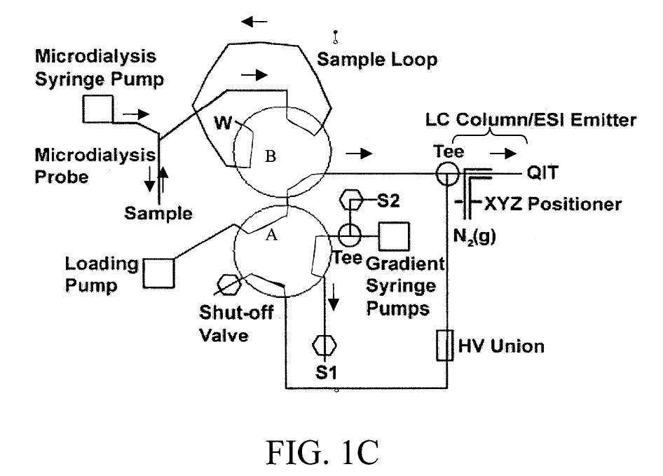

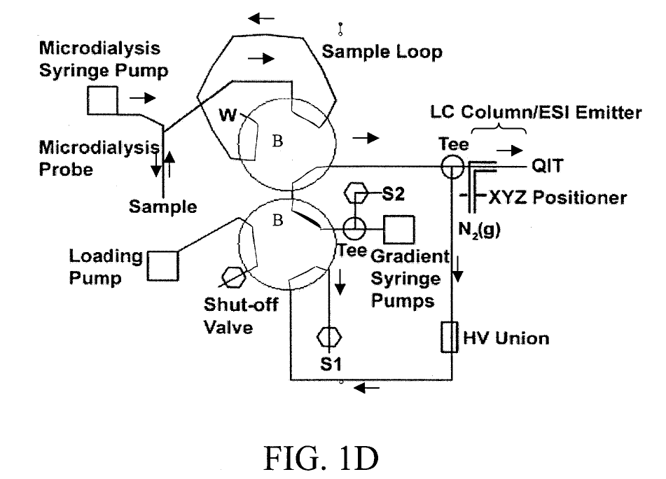

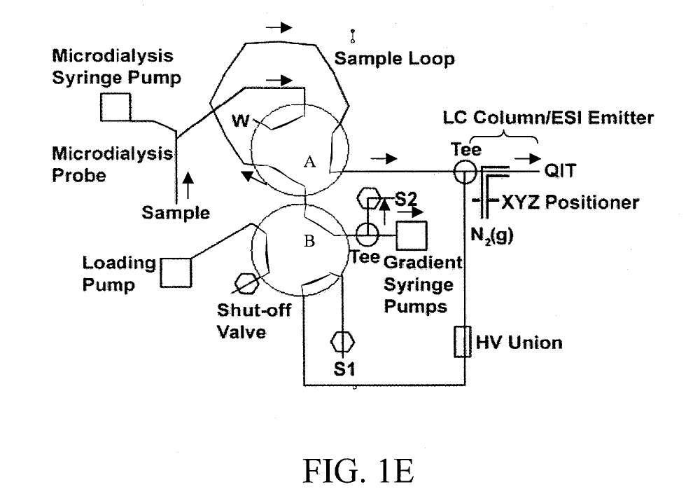

The apparatus can further include two or more valves. A first valve (valve 1) can be positioned as shown schematically in FIGS. 1A-1D and used to select the pump for separation or sample loading, and a second valve (valve 2) can serve as an injection valve. To perform injection of a sample, such as a dialysate, samples are loaded onto the sample loop of the injector valve (valve 2) either by a conventional syringe port or from the microdialysis probe, as shown in FIG. 1A. After the sample loop is filled, valve 2 is actuated to allow sample to be preconcentrated onto the integrated column. The preconcentrating pump is selected by valve 1 (shown in FIG. 1B) and sample is pumped onto the integrated column. Preferably, the sample is pumped at a rate of about 350 nL/min. to about 400 nL/min. More preferably, the sample is pumped at a rate of about 370 nL/min. After the sample is loaded, valve 2 can be actuated to remove the sample loop from the flow path and the column desalted (preferably at the preconcentration flow rate) (shown in FIG. 1C). Valve 1 can then be used to select the gradient syringe pump, and gradient elution can be initiated with a post-valve split. Splitter 1 is preferably open and splitter 2 is preferably closed during chromatographic separation to minimize the dwell time of the gradient.

Preferably, the apparatus further includes an emitter positioning means. The orifice of the integrated separation column-electrospray emitter can be automatically positioned at the inlet of the mass spectrometer by the positioning means. The positioning means can be any actuating apparatus that provides sufficient force to direct and move the integrated separation column-electrospray emitter such that the orifice of the emitter is adjacent to the inlet of the mass spectrometer, as shown schematically by the XYZ positioner shown in FIG. 1D. The distance between the orifice of the emitter and the inlet of the mass spectrometer is about 0.1 mm to about 5.0 mm at a voltage of 1.5 kv. Preferably, the distance is 0.5 mm from the emitter orifice to the mass spectrometer outlet. Preferably, the positioning means does not have the emitter positioned at the mass spectrometer inlet during desalting of the column and/or removal of salt droplets from the emitter orifice.

Optionally, the apparatus of the subject invention can include automating means, such as a computer, which controls the timing of the apparatus via a computer program or programs. The automating means can control one or more operations (e.g., valve control, desalting of integrated column-electrospray emitter) to influence one or more of the following parameters: the preconcentration time, desalting time, separation/electrospray time, and reequilibration time, for example.

In another aspect, the subject invention concerns a method for identifying and monitoring peptides in vivo by collecting and analyzing a sample using the apparatus of the present invention. Detection of multiple known peptides at low-attomole levels (low-picomolar concentrations) in complex samples was achieved by using the apparatus and method of the subject invention. The invention allows monitoring of endogenous neuropeptides at basal and stimulated levels with at least 30-min. temporal resolution in microdialysis samples. Unknown peptides can be sequenced at attomole levels, using time-segmented and data-dependent MS² and MS³ scans, and optionally correlated to neuropeptide precursors using database searching (Yates, J. R et al., Anal. Chem. 1998, 70, 3557-65; Perkins, D. N. et al., Electrophoresis 1999, 20, 3551-67.). Validation of peptide sequences was achieved by comparison of retention time, MS² spectra, and MS³ spectra with that of a synthetic peptide and by de novo sequence interpretation (Taylor, J. A. et al. A,nal. Chem. 2001, 73, 2594-04). The method of the subject invention is particularly useful for monitoring known peptides, studying processing of endogenous peptides, and characterizing novel peptides that are potentially neuroactive. The method of the subject invention includes collecting a dialysate sample, preconcentrating components (e.g., peptides) from the dialysate into an integrated separation column-electrospray emitter, chromatographically separating the components using the integrated separation column-electrospray emitter, and characterizing one or more of the components using a mass spectrometer that is in operable communication with the integrated separation column-electrospray emitter. The components (e.g., peptides) from dialysate can be preconcentrated and desalted on the integrated column-emitter using a first pump and, by switching to a second pump that is equilibrated to a lower pressure after preconcentration and desalting, separation and electrospray can be performed at a lower flow rate than the loading flow rate. In vivo monitoring of known peptides can carried out using the procedure described in Example 3. In vivo monitoring identification of previously unknown peptides can be carried out using the procedure described in Example 4. Optionally, mass spectra can be generated and correlated with protein databases for automated peptide sequencing and protein precursor identification.

Fused-silica capillary LC columns (25-μm i.d.) with 3-μm i.d. integrated electrospray emitters interfaced to a quadrupole ion trap mass spectrometer were evaluated for high-sensitivity LC-MS². Column preparation involved constructing frits by in situ photopolymerization of glycidyl methacrylate and trimethylolpropane trimethacrylate, preparing the electrospray emitter by pulling the column outlet to a fine tip with a CO₂ laser puller, and slurry-packing the column with 5-μm reversed-phase particles. Large-volume injections were facilitated by an automated two-pump system that allowed high-flow rates for sample loading and low-flow rates for elution. Small electrospray emitters, low elution flow rates, and optimization of gradient steepness allowed a detection limit of 4 amol, corresponding to 2 pM for 1.8 μL injected on-column, for a mixture of peptides dissolved in artificial cerebral spinal fluid. The system was coupled on-line to microdialysis sampling and was used to monitor and discover endogenous neuropeptides from the globus pallidus of anesthetized male Sprague-Dawley rats. Time-segmented MS² scans enabled simultaneous monitoring of Met-enkephalin, Leu-enkephalin, and unknown peptides. Basal dialysate levels of Met-enkephalin and Leu-enkephalin were 60±30 and 70±20 pM while K⁺-stimulated levels were 1900±500 and 1300±300 pM, respectively (n=7). Data-dependent and time-segmented MS² scans revealed several unknown peptides that were present in dialysate. One of the unknowns was identified as peptide I₁₋₁₀ (SPQLEDEAKE) (SEQ ID NO. 4), a novel product of preproenkephalin A processing, using MS², MS³, and database searching.

Using microdialysis, low-molecular weight compounds can be sampled from the extracellular space where the turnover of neurotransmitters is very fast. Using the method of the subject invention, a dialysate can be collected and analyzed from various anatomical regions of an organism. The method of the subject invention is particularly well suited for collecting and analyzing a dialysate from various regions of the brain, such as the striatum, hippocampus, and substantia nigra.

In a further aspect, the present invention relates to isolated or purified peptides that have been identified within the extracellular fluid of the rat brain using the method and apparatus of the subject invention. These peptides represent cleavage products of proenkephalin A (PEA), neurogranin, fibrinogen alpha chain precursor, fibrinogen beta chain precursor, excitatory amino acid transporter 1, and brain acidic membrane protein. The peptides of the subject invention include YGGFM (SEQ ID NO: 1) (Met-enkephalin), YPVEP (SEQ ID NO: 2), YPVEPEEE (SEQ ID NO: 3), SPQLEDEAKE (SEQ ID NO: 4), SPQLEDEAKELQ (SEQ ID NO: 5), VGRPEWWMDYQ (SEQ ID NO: 6), YGGFL (SEQ ID NO: 7) (Leu-enkephalin), YSKEVPEME (SEQ ID NO: 8), RKGPGPGGPGGAGGARGGAGGGPSGD (SEQ ID NO: 9), KGPGPGGPGGAGGARGGAGGGP (SEQ ID NO: 10), KGPGPGGPGGAGGARGGAGGGPSGD (SEQ ID NO: 1 1), GPGPGGPGGAGGARGGAGGGP (SEQ ID NO: 12), GPGPGGPGGAGGARGGAGGGPS (SEQ ID NO: 13), GPGPGGPGGAGGARGGAGGGPSGD (SEQ ID NO: 14), GPGGPGGAGGARGGAGGGPSGD (SEQ ID NO: 15), ADTGTTDEFIEAGGDIR (SEQ ID NO: 16), DTGTTDEFIEAGGDIR (SEQ ID NO: 17), EFIEAGGDIR (SEQ ID NO: 18), SPVPDLVPG (SEQ ID NO: 19), SQLQEGPPEWK (SEQ ID NO: 20), LVQTQAATDSDKVDLSIAR (SEQ ID NO: 21), TTDSDKVDLSIA (SEQ ID NO: 22) (Fibrinopeptide B₁₋₁₃), TDSDKVDLSIAR (SEQ ID NO: 23) (Fibrinopeptide B₂₋₁₄), IAQDNEPEKPVAKSETKM (SEQ ID NO: 24), QDNEPEKPVADSETKM (SEQ ID NO: 25), DNEPEKPVADSETKM (SEQ ID NO: 26), EPEKPVADSETKM (SEQ ID NO: 27), AKAPAPAAPAAEPQAEAPVAS (SEQ ID NO: 28), and AKAPAPAAPAAEPQAEAPVASSEQSVAVKE (SEQ ID NO: 29), SEQ ID NO: 31, SEQ ID NO: 32, SEQ ID NO: 33, SEQ ID NO: 34, SEQ ID NO: 35, SEQ ID NO: 36, and SEQ ID NO: 37, and biologically active or non-biologically fragments and variants of any of the foregoing (including homologues, e.g., mammalian homologues).

Met-enkephalin and Leu-enkephalin represent known cleavage products of PEA; however, the other peptides represent novel cleavage products of the above protein precursors. Specifically, peptides of SEQ ID NOs: 2-6 and 8 are cleavage products of PEA. Peptides of SEQ ID NOs: 9-15 are cleavage products of neurogranin. Peptides of SEQ ID NOs: 16-20 are cleavage products of fibrinogen alpha chain precursor. Peptides of SEQ ID NOs: 21-23 are cleavage products of fibrinogen beta chain precursor. Peptides of SEQ ID NOs: 24-27 are cleavage products of excitatory amino acid transporter 1. Peptides of SEQ ID NOs: 28 and 29 are cleavage products of brain acidic membrane protein.

The peptides of the subject invention that are cleavage products of preproenkephalin A (SEQ ID NO: 2, SEQ ID NO: 3, SEQ ID NO: 4, SEQ ID NO: 5, SEQ ID NO: 6, and SEQ ID NO: 8) have been observed to be co-released with the known neuropeptides YGGFM (SEQ ID NO: 1) (Met-enkephalin) and YGGFL (SEQ ID NO: 7) (Leu-enkephalin). Met- and leu-enkephalins compete with and mimic the effects of opiate drugs, playing a role in a number of physiological functions, including pain perception and responses to stress.

The subject invention further pertains to methods for increasing the levels of gamma-aminobutyric acid (GABA) and/or aspartate in vivo or in vitro by administration of either a peptide of the subject invention or a nucleotide sequence encoding the peptide, or a biologically active fragment or variant thereof. According to the methods of the present invention, nucleotide sequences encoding peptides of the invention can be administered to a host in vivo or in vitro as naked DNA, or within a recombinant construct, such as a viral vector or non-viral vector. Optionally, the recombinant construct can include regulatory sequences operably linked to the nucleotide sequences of the present invention, such as a promoter sequence capable of driving expression of the operably linked nucleotide sequence within a host cell in vitro and within a subject in vivo. Examples of host cells include prokaryotic and eukaryotic host cells, and particularly vertebrate cells, such as mammalian cells (e.g., human cells). The data disclosed herein establish that administration of the I11lo peptide (SEQ ID NO: 4) to a rat causes increases in concentrations of GABA, aspartate, and several other unknown compounds. Without being bound by theory, the I₁₋₁₀ peptide may cause an increase in the endogenous levels of these compounds in vivo by promoting their production, promoting their release, inhibiting their degradation, or a combination thereof. GABA is the primary inhibitory neurotransmitter in the central nervous system (CNS). By gating negative chloride (Cl⁻) ions into the interior of nerve cells, GABA inhibits the presynaptic release of neurotransmitter due to a positive voltage polarization pulse (Whiting, P. J., et al., Structure and pharmacology of vertebrate GABA_{A} receptor subtypes. In: Bradley, R. J., and Harris, R. A., eds. International Review of Neurobiology. San Diego: Academic Press, 1995, p. 95). GABA is capable of inducing relaxation, analgesia, and sleep in an individual. Barbiturates and benzodiazepines are known to stimulate GABA receptors, and thereby induce relaxation. Several neurological disorders, such as epilepsy, sleep disorders, and Parkinson's disease are affected by GABA. GABA is biosynthesized in the brain from glutamate and vitamin B6. Because GABA is a zwitterion (doubly-ionized amino acid), it cannot easily cross the blood-brain barrier, and effective methods of raising brain levels of GABA are desired. Novel GABA drugs (such as GABA receptor agonists and GABA facilitators) represent one of the most active areas of psychotropic research. For example, depakote (valproic acid) inhibits GABA-aminotransferase (GABA-T), the enzyme responsible for degrading GABA in the synapse, and seems to act on nerve membranes to reduce susceptibility to seizure. Gabapentine is an anti-epileptic (NEURONTIN) that is finding psychiatric application as a mood stabilizer, and may encourage production, or discourage degradation, of GABA.

The data disclosed herein indicate that the amino acid sequence of peptide I₁₋₁₀ (SEQ ID NO: 4) is contained within the human enkephalin precursor protein, NCBI Accession: EQHUA (Comb, M. et al., Nature, 1982, 295(5851):663-666), which differs by only two amino acids from the homologous enkephalin rat (Rattus norvegicus) precursor protein, NCBI Accession: EQRTA (EQRTA and PENK_rat refer to the same protein) (Rosen, H. et al., J. Biol. Chem., 1984, 259(22):14309-14313).

In another aspect, the present invention includes a host cell, or other recombinant construct, such as viral or non-viral vector, containing a heterologous nucleotide encoding a peptide of the present invention. The host cell can be a prokaryotic or eukaryotic cell that is transformed to express a peptide of the present invention. The terms “transform” and “genetically modify” are used interchangeably herein to refer to introduction of an exogenous polynucleotide sequence into a prokaryotic or eukaryotic cell by any means known in the art (including, for example, direct transmission of a polynucleotide sequence from a cell or virus particle, as well as transmission by infective virus particles and transmission by any other known means for introducing a polynucleotide into a cell), resulting in a permanent or temporary alteration of genotype and in an immortal or non-immortal cell line. The transformed hosts can be used, for example, to deliver nucleotide sequences of the present invention to a host in order to be expressed and raise endogenous levels of GABA and/or aspartate. Alternatively, the transformed hosts can be used as “biofactories” to produce the peptides of the present invention in large quantities.

The present invention also includes isolated or purified fragments of the peptides identified using the methods and apparatus of the present invention. As used herein, the term “fragments” means fragments comprising at least 5, 10, 15, 20, 25, 30, 35, 40, 50, 75, 100, or 150 consecutive amino acids of a peptide of the present invention to the extent that fragments of these lengths are consistent with the lengths of the particular peptides being referred to.

The present invention also provides for the exclusion of any fragments specified by N-terminal and C-terminal positions or by size in amino acid residues as described above. Any number of fragment species specified by N-terminal and C-terminal positions or sub-genus of fragments specified by size in amino acid residues as described above may be excluded from the invention. Fragments of the peptides of the present invention can be immediately envisioned using the above description and are therefore not individually listed solely for the purpose of not unnecessarily lengthening the specification.

The peptides of the present invention need not be biologically active since they would be useful, for example, in immunoassays, in epitope mapping, epitope tagging, as vaccines, to raise antibodies, stimulate an immune response in a heterologous species, and as molecular weight markers. The peptides of the present invention may be used to generate antibodies to a particular portion of the peptide. These antibodies can then be used in immunoassays well known in the art to distinguish between human and non-human cells and tissues or to determine whether cells or tissues in a biological sample are, or are not, of the same type which express the peptide of the present invention. The antibodies can be used to detect and/or isolate peptides within a sample, or larger epitope-containing sequences, including the full-length sequences, such as preproenkephalin A (PEA), neurogranin, fibrinogen alpha chain precursor, fibrinogen beta chain precursor, excitatory amino acid transporter 1, and brain acidic membrane protein, depending upon the particular peptide used to generate the antibody. Optionally, the antibody is polyclonal or monoclonal. Preferably, the peptide is an epitope-containing fragment of at least 8, 10, 12, 15, 20, 25, or 30 amino acids. As used herein, the term “antibody” refers to a polypeptide or group of polyeptides which are comprised of at least one binding domain, where an antibody binding domain is formed from the folding of variable domains of an antibody molecule to form three-dimensional binding spaces with an internal surface shape and charge distribution complementary to the features of an antigenic determinant of an antigen, which allows an immunological reaction with the antigen. Antibodies include recombinant proteins comprising the binding domains, as well as fragments, including Fab, Fab′, F(ab)₂, and F(ab)₂ fragments.

As used herein, the term “antigenic determninant” is the portion of an antigen molecule that determines the specificity of the antigen-antibody reaction. An “epitope” refers to an antigenic determinant of a peptide. An epitope can comprise as few as 3 amino acids in a spatial conformation which is unique to the epitope. Generally, an epitope consists of at least 6 such amino acids, and more usually at least 8-10 such amino acids. Methods for determining the amino acids which make up an epitope include x-ray crystallography, 2-dimensional nuclear magnetic resonance, and epitope mapping, e.g., Pepscan method, described by H. Mario Geysen et al., 1984, Proc. Natl. Acad. Sci. U.S.A., 81:3998-4002; PCT Publication Nos. WO 84/03564 and WO 84/03506.

Another aspect of the subject invention concerns an isolated nucleic acid sequence, e.g., DNA or mRNA, encoding a peptide of the subject invention, or fragment or variant thereof. In one embodiment, the nucleic acid sequence encodes a peptide selected from the group consisting of YGGFM (SEQ ID NO: 1) (Met-enkephalin), YPVEP (SEQ ID NO: 2), YPVEPEEE (SEQ ID NO: 3), SPQLEDEAKE (SEQ ID NO: 4), SPQLEDEAKELQ (SEQ ID NO: 5), VGRPEWWMDYQ (SEQ ID NO: 6), YGGFL (SEQ ID NO: 7) (Leu-enkephalin), YSKEVPEME (SEQ ID NO: 8), RKGPGPGGPGGAGGARGGAGGGPSGD (SEQ ID NO: 9), KGPGPGGPGGAGGARGGAGGGP (SEQ ID NO: 10), KGPGPGGPGGAGGARGGAGGGPSGD (SEQ ID, NO: 11), GPGPGGPGGAGGARGGAGGGP (SEQ ID NO: 12), GPGPGGPGGAGGARGGAGGGPS (SEQ ID NO: 13), GPGPGGPGGAGGARGGAGGGPSGD (SEQ ID NO: 14), GPGGPGGAGGARGGAGGGPSGD (SEQ ID NO: 15), ADTGTTDEFIEAGGDIR (SEQ ID NO: 16), DTGTTDEFIEAGGDIR (SEQ ID NO: 17), EFIEAGGDIR (SEQ ID NO: 18), SPVPDLVPG (SEQ ID NO: 19), SQLQEGPPEWK (SEQ ID NO: 20), LVQTQAATDSDKVDLSIAR (SEQ ID NO: 21), TTDSDKVDLSIA (SEQ ID NO: 22) (Fibrinopeptide B-₁₋₁₃), TDSDKVDLSIAR (SEQ ID NO: 23) (Fibrinopeptide B₂₋₁₄), IAQDNEPEKPVAKSETKM (SEQ ID NO: 24), QDNEPEKPVADSETKM (SEQ ID NO: 25), DNEPEKPVADSETKM (SEQ ID NO: 26), EPEKPVADSETKM (SEQ ID NO: 27), AKAPAPAAPAAEPQAEAPVAS (SEQ ID NO: 28), AKAPAPAAPAAEPQAEAPVASSEQSVAVKE (SEQ ID NO: 29), MDELYPVEPEEEANGEILA (SEQ ID NO: 31), FAESLPSDEEGESYSKEVPEME (SEQ ID NO: 32), MAQFLRLCIWLLALGSCLLATVQADCSQDCAKCSYRLVRPGDINFLACTLECEGQLP SFKIWETCKDLLQVSKPEFPWDNIDMYKDSSKQDESHLLAKKYGGFMKRYGGFMK KMDELYPVEPEEEANGEILAKRYGGFMKKDADEGDTLANSSDLLKELLGTGDNRA KD SHQQESTNND EDSTSKRYGGFMRGL KRSPQLEDEAKELQKR YGGFMRRVGPEWWMDYQKRYGGFLKRFAESLPSDEEGESYSKEVPEME KR YGGFMR (SEQ ID NO: 33), KKMDELYPVEPEEEANGEILAKRYGGFMKKDADEGDTLANS SDLLKELLGTGDNR AKD SHQQESTNND EDSTSKRYGGFMRGLKRSPQLEDEAKELQKR (SEQ ID NO: 34), KRSPQLEDEAKELQKR (SEQ ID NO: 35), KRSPQLEDEAKELQKRYGGFMRRVGPEWWMDYQKRYGGFLKRFAESLPSDEEGES YSKEVPEMEKR (SEQ ID NO: 36); and KRYGGFLKRFAESLPSDEEGESYSKEVPEMEKR (SEQ ID NO: 37), and biologically active or non-biologically fragments and variants of any of the foregoing (including homologues, e.g., mammalian homologues).

The peptides of the present invention also include the following hypothetical intermediates (HIs): MDELYPVEPEEEANGEILA (SEQ ID NO: 31); FAESLPSDEEGESYSKEVPEME (SEQ ID NO: 32); MAQFLRLCIWLLALGSCLLATVQADCSQDCAKCSYRLVRPGDINFLACTLECEGQLP SFKIWETCKDLLQVSKPEFPWDNIDMYKDS SKQDESHLLAKKYGGFMKRYGGFMK KMDELYPVEPEEEANGEILAKRYGGFMKKDADEGDTLANS SDLLKELLGTGDNRA KD SHQQESTNND EDSTSKRYGGFMRGL KRSPQLEDEAKELQKR YGGFMRRVGPEWWMDYQKRYGGFLKRFAESLPSDEEGESYSKEVPEME KR YGGFMR (SEQ ID NO: 33); KKMDELYPVEPEEEANGEILAKRYGGFMKKDADEGDTLANS SDLLKELLGTGDNR AKD SHQQESTNND EDSTSKRYGGFMRGLKRSPQLEDEAKELQKR (SEQ ID NO: 34); KRSPQLEDEAKELQKR (SEQ ID NO: 35); KRSPQLEDEAKELQKRYGGFMRRVGPEWWMDYQKRYGGFLKRFAESLPSDEEGES YSKEVPEMEKR (SEQ ID NO: 36); KRYGGFLKRFAESLPSDEEGESYSKEVPEMEKR (SEQ ID NO: 37), and fragments or variants of any of the foregoing. The peptides of SEQ ID NO: 31 and SEQ ID NO: 32 are intermediates without mono- and di-basic amino acids, as shown in FIG. 26. The peptides of SEQ ID NOs: 33-37 are intermediates with mono- and di-basic amino acids, as shown in FIG. 26.

The nucleic acid sequences can consist of the same nucleotides (and codons) present in the relevant portion of the native coding sequence of the corresponding protein precursor (e.g., PEA, neurogranin, fibrinogen alpha chain precursor, fibrinogen beta chain precursor, excitatory amino acid transporter 1, brain acidic membrane protein). However, it should be understood that nucleic acid sequences encoding peptides of the present invention can vary due to the degeneracy of the genetic code.

Nucleotide sequence, polynucleotide or nucleic acid are understood to mean, according to the present invention, either a double-stranded DNA, a single-stranded DNA or products of transcription of the said DNAs (e.g., RNA molecules). The nucleic acid, polynucleotide, or nucleotide sequences of the invention have been isolated, purified (or partially purified), by separation methods including, but not limited to, ion-exchange chromatography, molecular size exclusion chromatography, affinity chromatography, or by genetic engineering methods such as amplification, cloning or subcloning.

As used herein, the term “homology” refers to comparisons between protein and/or nucleic acid sequences and is evaluated using any of the variety of sequence comparison algorithms and programs known in the art. Both peptide and nucleic acid sequence homologies may be evaluated using such algorithms and programs. Such algorithms and programs include, but are by no means limited to, TBLASTN, BLASTP, FASTA, TFASTA, and CLUSTALW (Pearson and Lipman, 1988, Proc. Natl. Acad. Sci. USA 85(8):2444-2448; Altschul el al. [1990] J. Mol. Biol. 215(3):403-410; Thompson et al. [1994] Nucleic Acids Res. 22(2):4673-80; Higgins et al. [1996] Methods Enzymnol. 266:383-402; Altschul et al. [1990] J. Mol. Biol. 215(3):403-410; Altschul et al. [1993] Nature Genetics 3:266-272). In a particularly preferred embodiment, peptide and nucleic acid sequence are evaluated using the Basic Local Alignment Search Tool (“BLAST”), which is well known in the art (see, e.g., Karlin and Altschul, 1990, Proc. Natl. Acad. Sci. U.S.A. 87:2267-2268; Altschul et al., 1990, J. Mol. Biol. 215:403-410; Altschul et al., 1993, Nature Genetics 3:266-272; Altschul et al., 1997, Nuc. Acids Res. 25:3389-3402). In particular, five specific BLAST programs are used to perform the following task:

(1) BLASTP and BLAST3 compare an amino acid query sequence against a protein sequence database;

(2) BLASTN compares a nucleotide query sequence against a nucleotide sequence database;

(3) BLASTX compares the six-frame conceptual translation products of a query nucleotide sequence (both strands) against a protein sequence database;

(4) TBLASTN compares a query protein sequence against a nucleotide sequence database translated in all six reading frames (both strands); and

(5) TBLASTX compares the six-frame translations of a nucleotide query sequence against the six-frame translations of a nucleotide sequence database.

The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., 1992, Science 256:1443-1445; Henikoff and Henikoff, 1993, Proteins 17:49-61). Less preferably, the PAM or PAM250 matrices may also be used (see, e.g., Schwartz and Dayhoff, eds., 1978, Matrices for Detecting Distance Relationships: Atlas of Protein Sequence and Structure, Washington: National Biomedical Research Foundation). The BLAST programs evaluate the statistical significance of all high-scoring segment pairs identified, and preferably selects those segments which satisfy a user-specified threshold of significance, such as a user-specified percent homology. Preferably, the statistical significance of a high-scoring segment pair is evaluated using the statistical significance formula of Karlin (see, e.g., Karlin and Altschul, 1990, Proc. Natl. Acad. Sci. USA 87:2267-2268).

A homologous nucleotide sequence, for the purposes of the present invention, encompasses a nucleotide sequence having a percentage identity with the bases of the nucleotide sequences of between at least (or at least about) 20.00% to 99.99% (inclusive). The aforementioned range of percent identity is to be taken as including, and providing written description and support for, any fractional percentage, in intervals of 0.01%, between 20.00% and, up to, including 99.99%. These percentages are purely statistical and differences between two nucleic acid sequences can be distributed randomly and over the entire sequence length.

In various embodiments, homologous sequences exhibiting a percentage identity with the bases of the nucleotide sequences of the present invention can have 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identity with the polynucleotide sequences of the instant invention.

The subject invention also provides nucleotide sequences complementary to the sequences disclosed herein. Thus, the invention is understood to include any DNA whose nucleotides are complementary to those of the sequence of the invention, and whose orientation is reversed (e.g., anti-sense sequences).

The present invention further comprises fragments of the sequences of the instant invention as well as fragments of the gene products contained within the polynucleotide sequences provided herein. Representative fragments of the polynucleotide sequences according to the invention will be understood to mean any nucleotide fragment having at least 8 successive nucleotides, preferably at least 12 successive nucleotides, and still more preferably at least 15 or at least 20 successive nucleotides of the sequence from which it is derived. The upper limit for such fragments is the total number of polynucleotides found in the full length sequence (or, in certain embodiments, of the full length open reading frame (ORF) identified herein). It is understood that such fragments refer only to portions of the disclosed polynucleotide sequences that are not listed in a publicly available database.

In some embodiments, the subject invention includes those fragments capable of hybridizing under stringent conditions with a nucleotide sequence according to the invention. Hybridization under conditions of high or intermediate stringency, are defined below. Thus, conditions are chosen such that they allow hybridization to be maintained between two complementary DNA fragments.

Various degrees of stringency of hybridization can be employed. The more severe the conditions, the greater the complementarity that is required for duplex formation. Severity of conditions can be controlled by temperature, probe concentration, probe length, ionic strength, time, and the like. Preferably, hybridization is conducted under moderate to high stringency conditions by techniques well known in the art, as described, for example, in Keller, G. H., M. M. Manak (1987) DNA Probes, Stockton Press, New York, N.Y., pp. 169-170.

Examples of various stringency conditions are provided herein. Hybridization of immobilized DNA on Southern blots with 32P-labeled gene-specific probes can be performed by standard methods (Maniatis et al. (1982) Molecular Cloning: A Laboratory Manual, Cold Spring Harbor Laboratory, New York.). In general, hybridization and subsequent washes can be carried out under moderate to high stringency conditions that allow for detection of target sequences with homology to the exemplified polynucleotide sequence. For double-stranded DNA gene probes, hybridization can be carried out overnight at 20-25° C. below the melting temperature (Tm) of the DNA hybrid in 6×SSPE, 5× Denhardt's solution, 0.1% SDS, 0.1 mg/ml denatured DNA. The melting temperature is described by the following formula (Beltz et al., 1983, Methods of Enzymology, R. Wu, L. Grossman and K. Moldave [eds.] Academic Press, New York 100:266-285).

Tm=81.5° C.+16.6 Log[Na+]+0.41(% G+C)−0.61(% formamide)−600/length of duplex in base pairs.

Washes are typically carried out as follows:

(1) twice at room temperature for 15 minutes in 1×SSPE, 0.1% SDS (low stringency wash);

(2) once at Tm−20° C. for 15 minutes in 0.2×SSPE, 0.1% SDS (moderate stringency wash).

For oligonucleotide probes, hybridization can be carried out overnight at 10-20° C. below the melting temperature (Tm) of the hybrid in 6×SSPE, 5×Denhardt's solution, 0.1% SDS, 0.1 mg/ml denatured DNA. Tm for oligonucleotide probes can be determined by the following formula: Tm (0° C.)=2(number T/A base pairs) +4(number G/C base pairs) (Suggs et al. [1981] ICN-UCLA Symp. Dev. Biol. Using Purified Genes, D. D. Brown [ed.], Academic Press, New York, 23:683-693).

Washes can be carried out as follows:

(1) twice at room temperature for 15 minutes 1×SSPE, 0.1% SDS (low stringency wash;

(2) once at the hybridization temperature for 15 minutes in 1×SSPE, 0.1% SDS (moderate stringency wash).

In general, salt and/or temperature can be altered to change stringency. With a labeled DNA fragment >70 or so bases in length, the following conditions can be used:

| | |
|---|---|
| Low: | 1 or 2X SSPE, room temperature |
| Low: | 1 or 2X SSPE, 42° C. |
| Moderate: | 0.2X or 1X SSPE, 65° C. |
| High: | 0.1X SSPE, 65° C. |

Duplex formation and stability depend on substantial complementarity between the two strands of a hybrid and, as noted above, a certain degree of mismatch can be tolerated.

Another aspect of the invention provides vectors for the cloning and/or the expression of a polynucleotide sequence taught herein. Vectors of this invention can also comprise elements necessary to allow the expression and/or the secretion of the nucleotide sequences in a given prokaryotic or eukaryotic host cell. The vector can contain a promoter, signals for initiation and for termination of translation, as well as appropriate regions for regulation of transcription. In certain embodiments, the vectors can be stably maintained in the host cell and can, optionally, contain signal sequences directing the secretion of translated protein. These different elements are chosen according to the host cell used. Vectors can integrate into the host genome or, optionally, be autonomously-replicating vectors.

The subject invention also provides for the expression of a polypeptide, peptide, derivative, or analog disclosed herein. The disclosed sequences can also be regulated by a second nucleic acid sequence so that the protein or peptide is expressed in a host transformed with the recombinant DNA molecule. For example, expression of a protein or peptide may be controlled by any promoter/enhancer element known in the art. Promoters which may be used to control expression include, but are not limited to, the CMV promoter, the SV40 early promoter region (Bernoist and Chambon [1981] Nature 290:304-310), the promoter contained in the 3′ long terminal repeat of Rous sarcoma virus (Yamamoto et al. [1980] Cell 22:787-97), the herpes thymidine kinase promoter (Wagner et al. [1981] Proc. Natl. Acad. Sci. U.S.A. 78:1441-1445), the regulatory sequences of the metallothionein gene (Brinster et al. [1982] Nature 296:39-42); prokaryotic vectors containing promoters such as the β-lactamase promoter (Villa-Kamaroff et al. [1978] Proc. Natl. Acad. Sci. U.S.A. 75:3727-3731), or the tac promoter (DeBoer et al. [1983] Proc. Natl. Acad. Sci. U.S.A. 80:21-25); see also, “Useful proteins from recombinant bacteria” [1980] Scientific American 242:74-94; plant expression vectors comprising the nopaline synthetase promoter region (Herrera-Estrella et al. [1983] Nature 303:209-213) or the cauliflower mosaic virus 35S RNA promoter (Gardner et al. [1981] Nucl. Acids Res. 9:2871), and the promoter of the photosynthetic enzyme ribulose biphosphate carboxylase (Herrera-Estrella et al. [1984] Nature 310:115-120); promoter elements from yeast or fungi such as the Gal 4 promoter, the ADC (alcohol dehydrogenase) promoter, PGK (phosphoglycerol kinase) promoter, and/or the alkaline phosphatase promoter

The vectors according to the invention are, for example, vectors of plasmid or viral origin. In a specific embodiment, a vector is used that comprises a promoter operably linked to a protein or peptide-encoding nucleic acid sequence contained within the disclosed polynucleotide sequences, one or more origins of replication, and, optionally, one or more selectable markers (e.g., an antibiotic resistance gene). Expression vectors comprise regulatory sequences that control gene expression, including gene expression in a desired host cell. Exemplary vectors for the expression of the polypeptides of the invention include the pET-type plasmid vectors (PROMEGA) or pBAD plasmid vectors (INVITROGEN) or those provided in the examples below. Furthermore, the vectors according to the invention are useful for transforming host cells so as to clone or express the nucleotide sequences of the invention.

As indicated above, the invention also encompasses the host cells transformed by a vector according to the invention. These cells may be obtained by introducing into host cells a nucleotide sequence inserted into a vector as defined above, and then culturing the cells under conditions allowing the replication and/or the expression of the transfected nucleotide sequence.

The host cell may be chosen from eukaryotic or prokaryotic systems, such as for example bacterial cells, (Gram negative or Gram positive), yeast cells, animal cells (such as Chinese hamster ovary (CHO) cells), plant cells, and/or insect cells using baculovirus vectors. In some embodiments, the host cells for expression of the polypeptides include, and are not limited to, those taught in U.S. Pat. Nos. 6,319,691; 6,277,375; 5,643,570; or 5,565,335, each of which is incorporated by reference in its entirety, including all references cited within each respective patent.

Furthermore, a host cell strain may be chosen which modulates the expression of the inserted sequences, or modifies and processes the gene product in the specific fashion desired. Expression from certain promoters can be elevated in the presence of certain inducers; thus, expression of the genetically engineered polypeptide may be controlled. Furthermore, different host cells have characteristic and specific mechanisms for the translational and post-translational processing and modification (e.g., glycosylation, phosphorylation) of proteins. Appropriate cell lines or host systems can be chosen to ensure the desired modification and processing of the foreign protein expressed. For example, expression in a bacterial system can be used to produce an unglycosylated core protein product. Expression in yeast will produce a glycosylated product. Expression in mammalian cells can be used to ensure “native” glycosylation of a heterologous protein. Furthermore, different vector/host expression systems may effect processing reactions to different extents.

In other specific embodiments, the polypeptides, peptides or derivatives, or analogs thereof may be expressed as a fusion, or chimeric protein product (comprising the protein, fragment, analog, or derivative joined via a peptide bond to a heterologous protein sequence (e.g., a different protein)). Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer.

The peptides disclosed herein, or nucleotide sequences encoding the peptides, may be administered individually or in the form of a “cocktail” comprising at least two or more peptides according to the invention. The composition administered to the subject may, optionally, contain an adjuvant and may be delivered to the subject in any manner known in the art for the delivery of an active agent to a subject. Compositions may be formulated in any carriers, including for example, carriers described in E. W. Martin's Remington's Pharmaceutical Science, Mack Publishing Company, Easton, Pa.

The subject invention further concerns antibodies sensitive to the peptides of the subject invention, and the use of such antibodies in diagnostic and therapeutic applications.

Compositions comprising the subject peptides can include a pharmaceutically acceptable carrier, e.g., saline. The pharmaceutically acceptable carriers are well known in the art and also are commercially available. For example, such acceptable carriers are described in E. W. Martin's Remington's Pharmaceutical Science, Mack Publishing Company, Easton, Pa.

The apparatus and method of the subject invention can be utilized to obtain and analyze a biological sample from a wide variety of organisms (subjects). Likewise, the nucleic acids and peptides of the subject invention can be administered to a wide variety of organisms (subjects) to increase endogenous levels of GABA and/or aspartate. Mammalian species that can be the subjects of the methods and apparatus of the subject invention include, and are not limited to, apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. In addition, non-mammalian vertebrates (such as reptiles, avians, and amphibians) and invertebrate organisms can also serve as subjects for the methods and apparatus of the subject invention, providing the necessary biological sample or serving as the subject for in vivo or in vitro increase of GABA and/or aspartate, for example.

As used herein, “biologically active” peptides refers to those peptides capable of modulating (increasing or decreasing) the levels and/or activities of endogenous neurotransmitters when administered to an organism in effective amounts. For example, those peptides capable of increasing the levels and/or activity of GABBA and/or aspartate when administered to an organism in effective amounts are biologically active.

As used herein, the terms “peptide”, “polypeptide”, and “protein” are used interchangeably to refer to an amino acid sequence (i.e., a polymer of amino acids) of any length. These terms do not specify or exclude chemical or post-expression modifications of the polymer of amino acids, although chemical or post-expression modifications of these amino acid sequences may be included or excluded as specific embodiments. For example, peptides that include covalent attachment of glycosyl groups, acetyl groups, phosphate groups, lipid groups, and the like, are expressly encompassed by the terms “peptide”, “polypeptide”, and “protein”. Also included within the definition are peptides which contain one or more analogs of an amino acid (including, for example, non-naturally occurring amino acids, amino acids which only occur naturally in an unrelated biological system, modified amino acids from mammalian systems, etc.), peptides with unsubstituted linkages, as well as other modifications known in the art, both naturally occurring and non-naturally occurring.

As used herein, the term “isolated” requires that the material be removed from its original environment (e.g., the natural environment, if it is naturally occurring).

The term “purified” does not require absolute purity; rather, it is intended as a relative definition. Purification of starting material or natural material to at least one order of magnitude, preferably two or three orders, and more preferably four or five orders of magnitude is expressly contemplated.

The term “purified” is used herein to describe a polynucleotide of the invention, polynucleotide vector of the invention, or peptide of the invention, which has been separated from other compounds including, but not limited to, nucleic acids, lipids, carbohydrates, and proteins (such as the enzymes used in the synthesis of the polynucleotide, or the separation of covalently closed polynucleotides from linear polynucleotides). A polynucleotide is substantially pure when at least about 50%, preferably 60% to 75% of a sample exhibits a single polynucleotide sequence and conformation (linear versus covalently closed). A substantially pure polynucleotide typically comprises about 50%, preferably 60% to 90% weight/weight of a nucleic acid sample, more usually about 95%, and preferably is over about 99% pure. Polynucleotide purity or homogeneity is indicated by a number of means well known in the art, such as agarose or polyacrylamide gel electrophoresis of a sample, followed by visualizing a single polynucleotide band upon staining the gel. For certain purposes, higher resolution can be provided by using HPLC or other means well known in the art. A polypeptide is substantially pure when at least about 50%, preferably 60% to 75%, of a sample exhibits a single polypeptide sequence. A substantially pure polypeptide typically comprises about 50%, preferably 60% to 90% weight/weight of a protein sample, more usually about 95%, and preferably is over about 99% pure. Polypeptide purity or homogeneity is indicated by a number of means well known in the art, such as agarose or polyacrylamide gel electrophoresis of a sample, followed by visualizing a single polypeptide band upon staining the gel. For certain purposes, high resolution can be provided by using HPLC or other means well known in the art.

The terms “comprising”, “consisting of”, and “consisting essentially of” are defined according to their standard meaning. The terms may be substituted for one another throughout the instant application in order to attach the specific meaning associated with each term.

As used herein, the term “biological sample” is intended to mean any fluid of biological origin that can be separated and characterized using the methods and apparatus of the invention. The sample can be collected and immediately separated and characterized in a real-time fashion, or can be stored prior to analysis, for example. Preferably, the sample is extracellular fluid. More preferably, the sample is extracellular fluid obtained from the brain of a subject. A “standard” for a biological sample is a synthetic or naturally occurring fluid that can be used as a control or standard for a particular type of sample.

As used herein, the term “sample loading” including loading the sample loop of the injection valve of the apparatus.

As used herein, the term “preconcentration” refers to the action of preconcentrating components (e.g., peptides) from dialysate onto the separation means (e.g., CLC column). For example, peptides preconcentrate in weak (low organic) mobile phases by hydrophobic interactions with the C18 stationary phase (reverse-phase gradient elution).

As used herein, the term “desalt” refers to the action of removing weakly bound molecules (e.g., salts) from the separation means (e.g., CLC column) that could potentially interfere with the analysis of strongly bound molecules (e.g., peptides) by rinsing. For example, salts may be removed while peptides are retained during rinsing with a weak (low organic) mobile phase (reverse-phase gradient elution).

As used herein, the term “separating” refers to eluting strongly bound molecules (e.g., peptides) from the separation means (e.g., CLC column). For example, peptides of varying hydrophobicity may be separated and eluted from the CLC column by slowly increasing the strength (organic content) of the mobile phase (reverse-phase gradient-elution).

General concepts concerning mass spectrometry, particularly with regard to characterization of peptides and proteins are described in Russell, D. H. and Edmondson, R. D. J. Mass. Spect., 1997, 32:263-276), the contents of which is incorporated herein by reference in its entirety.

### Materials and Methods

Chemicals and Reagents. CLC solvents were purchased from BURDICK & JACKSON (Muskegon, Mich.). Peptides were from SIGMA (St. Louis, Mo.). Acetic acid and hydrofluoric acid were purchased from FISHER SCIENTIFIC (Pittsburgh, Pa.). Ethanol was purchased from J. T. Baker (Phillipsburg, N.J.). The following chemicals were purchased from ALDRICH (Milwaukee, Wis.) to prepare macroporous photopolymer frits: isooctane, toluene, trimethlolpropane trimethacrylate, glycidyl methacrylate, and benzoin methylether. Artificial cerebral spinal fluid (aCSF) used for microdialysis perfusion was composed of 145 mM NaCl, 2.68 mM KCl, 1.10 mM MgSO₄, and 1.22 mM CaCl₂. The high-K⁺, low-Na⁺ perfusate solution for depolarization experiments consisted of 145 mM KCl and 2.62 mM NaCl with other salts the same as aCSF. Mobile phases and aCSF were prepared weekly and were filtered with 20-nm-pore size aluminum oxide filters (FISHER) to remove particulates. Tryptic digests were purchased from MICHROM BIORESOURCES (Auburn, Calif.).

In Vivo Microdialysis. Commercial microdialysis probes (CMA/10 with 20,000 molecular weight cutoff, CMA, Solna, Sweden) with 4-mm active lengths were used (Maidment, N. T. et al., J. Neuroscience, 1989, 33:549-557; Shen, H. et al., J. Chromatogr., 1997, 704:43-52; Shen, H. et al., Ansl. Chem., 1999, 71:987-994; Emmett, M. R. et al., i J. Neurosci. Methods, 1995, 62:141-147; Andren, P. E. et al., Brain Res., 1999, 845:123-129; Maidment, N. T. et al., Microdialysis in the Neurosciences, Robinson, T. E. et al., Eds, Elsevier: Amsterdam, 1991, pp. 275-304). The outlet tubing of the microdialysis probe was replaced with fused-silica capillary (50-μm i.d., 360-μm o.d., POLYMICRO TECHNOLOGIES, Phoenix, Ariz.) to minimize dead volumes. Prior to use, microdialysis probes were rinsed with 70% ethanol at a flow rate of 0.6 μL/min. per the manufacturer's instructions. Deuterated Leu-enkephalin, where five nonexchangeable deuterium atoms were incorporated into the phenylalanine ring, was used to calculate the relative recovery through the microdialysis probe. In vitro relative recovery of 600 pM deuterated Leu-enkephalin in aCSF was 51±16% (n=10) at 37° C. Microdialysis probes were implanted and targeted to the globus pallidus using procedures described elsewhere (Shen, H. et al., J. Chromatogr., 1997, 704:43-52). The microdialysis probe was implanted into the striatum region of the rat brain with the following coordinates relative to the bregma region: anterior-posterior=−1.0 cm, medial-lateral=+0.17 cm and dorsal-ventral=−0.91 cm. Correct placement of the microdialysis probe was confirmed by inspection of cresyl violet-stained tissue sections cut by a cryomicrotome.

Preparation of CLC Columns with Integrated Electrospray Emitters. To prepare columns, a 1-cm section of polyimide was removed from a 20-cm length of 25 μm i.d. fused silica with a flame. The capillary was loaded with a solution of glycidyl methacrylate (7% v/v) and trimethylolpropane trimethacrylate (16% v/v), using He pressure and was placed inside PEEK sleeves (UPCHURCH SCIENTIFIC, Oak Harbor, Wash.) that formed a mask that exposed a ˜100-μm length of the bare fused silica. Polymerization was achieved by illuminating the exposed region of the capillary with a UV lamp (Spectronics, Westbury, N.Y.) for 30 minutes (Viklund, C. et al., Chem. Mater., 1997, 9:463-467; Chen, J. R. et al., Anal. Chem., 2000, 72(6):1224-1227). (In practice, 10 capillary frits were prepared simultaneously). Frits were flushed with acetone and were dried for storage by passing He through the capillaries for 5 minutes at 500 psi. A P-2000 CO₂ laser puller (SUTTER INSTRUMENTS, Novato, Calif.) was used to create integrated electrospray emitters of <1 cm beyond the frit. The settings used on the P-2000 were heat 350, filament 0, velocity 20, delay 135, and pull 125. The program was cycled 3-4 times to generate pulled capillary columns with <1-μm orifices. The resulting pulled columns were etched with 50% hydrofluoric acid (use extreme caution and neutralize with CaCl₂) (Valaskovic, G. A. et al., Anal. Chem., 1995, 67(20):3802-3805) for 30 seconds to create sharp-edged electrospray emitters with ˜3-μm i.d. Pulled columns were packed with an acetone slurry (10 mg/mL) of 5-μm Alltima C18 reversed-phase particles (ALLTECH, Deerfield, Ill.) at 1000 psi as described elsewhere (Valaskovic, G. A. et al., Anal. Chem., 1995, 67(20):3802-3805). Only 2 cm of the total 13-cm length of fused silica was packed, unless stated otherwise. The void at the head of the column did not contribute to extracolumn band broadening because all samples were injected in weak mobile phases to allow the analytes to stack at the head of the column. The CLC-MS² apparatus and its operation are described in Example 1 and FIGS. 1A-1E.

Spectral Reproducibility. Spectral reproducibility describes the % of the MS² spectra within any two CLC-MS² datasets (n and n+1) that are the same for a given precursor ion mass tolerance (e.g., 2.5 Da) and product ion threshold (e.g., 38% correlation). Spectral reproducibility was determined by the following formula:

Spectral Reproducibility=100(n−[(n+1)−n]/n)

where n is the number of MS² spectra for the 1^{st} dataset, (n+1) is the number of MS² spectra for the 2^{nd} dataset and [(n+1)−n] is the number of MS² spectra remaining following subtractive analysis of the two datasets.

Subtractive Analysis. Subtractive analysis is a method whereby the difference MS² spectra between any two CLC-MS² datasets can be determined for a given precursor ion mass tolerance and product ion threshold. Subtractive analysis was performed by the SEQUEST program IONQUEST (a binning algorithm) (Yates J. R. et al., Analytical Chemistry 70:3557-3565, 1998). This algorithm is usually used to subtract known contaminants (e.g., trypsin autolysis products and plasticizers) from CLC-MS² datasets prior to peptide sequencing and protein precursor identification by SEQUEST. In this work, a precursor ion mass tolerance of 2.5 Da and a product ion threshold of 38% correlation (i.e., the default values) were used to 1) determine spectral reproducibility and 2) determine MS² spectra observed only during depolarization.

Peptide Sequencing and Protein Precursor Identification. The SEQUEST Xcorr (cross correlation) score is a measure of the similarity between the m/z of the product ions observed in the MS² spectrum and the product ions predicted for peptide sequences generated by in silico proteolysis of the protein database. The normalized difference between the 1^{st}-ranked and 2^{nd}-ranked peptide sequences is the SEQUEST ΔCn (delta normalized correlation) score. Xcorr scores greater than 2.0 and ΔCn scores greater than 0.1 are considered significant (Ducret A. et al., Protein Science 7:706-719, 1998). However, it was recently shown that SEQUEST Xcorr scores below 2.5 for doubly-charged precursor ions and below 3.0 for triply-charged precursor ions suggest that false-positives will be present (Peng J. M. et al., Journal of Proteome Research 2:43-50, 2003; MacCross M. J. et al., Analytical Chemistry 74:5593-5599, 2002). Low Xcorr values were expected for non-tryptic peptides at attomole levels.

In order to minimize false-positives, a six step data-reduction strategy incorporating database searching, automated (cle novo sequencing and library searching was investigated for peptide sequencing and protein precursor identification. The data-reduction strategy in vivo and definitions characterizing the MS² spectra remaining after each step of data-reduction (1-6) are summarized below. The strategy involves sequential application of criteria, each set of which provides increasing selectivity.

Six Step Data-Reduction Strategy.

1. MS² spectra collected with a total ion current greater than 5e+5.

2. MS² spectra remaining from step 1 following subtraction of MS² spectra collected under basal conditions (with artificial CSF perfusing the probe) from MS² spectra collected during localized depolarization evoked by infusion of high K⁺, low Na⁺ solution through the dialysis probe. These MS² spectra were considered depolarization-specific.

a. Subtractive analysis was performed by the SEQUEST program IONQUEST (a binning algorithm), as described above.

b. Database searching programs (SEQUEST ver. 1.2 and Mascot ver. 1.8 (Perkins D. N. et al., Electrophoresis 20:3551-3567, 1999) correlated uninterpreted MS² spectra with the NCBI nonredundant protein database for Rattus norvegicus (12670 sequences). Precursor ion and product ion mass tolerances were 1.4 and 0.0 for Sequest and 2.0 and 0.8 for Mascot, respectively, as suggested by the manufacturers (the programs have different scoring systems). No protease specificity was selected. Post-translational modifications (PTMs) were investigated by performing error tolerant searches with Mascot.

3. MS² spectra remaining from step 2 with a SEQUEST ΔCn score greater than 0.1 were considered significant.

4. MS² spectra remaining from step 3 that met the following criteria were considered validated by database searching.

a. The 1^{st} ranked SEQUEST-derived sequence matched the 1^{st} ranked Mascot-derived sequence with a SEQUEST ΔCn score greater than 0.1 and a Mascot score indicating homology with greater than 95% probability.

b. More than one peptide was observed from each protein precursor.

5. MS² spectra remaining from step 4 were evaluated by automated de novo sequencing using the LUTEFISK program (ver. 1.3.2) (Taylor J. A. et al., Analytical Chemistry 73:2594-2604 (2001). A precursor ion tolerance of 2.0 and a product ion tolerance of 0.8 were employed (consistent with Mascot). If the 1^{st} ranked de novo-derived partial sequence matched the 1^{st} ranked database-derived sequence with greater homology than it matched the 2^{nd} ranked database-derived sequence peptide sequence, then the MS² spectrum was considered validated by de novo sequencing.

6. MS² remaining from step 3 were examined for incorrect assignments by the database searching programs. This done by searching the MS² spectra from step 4 against a library of MS² spectra made from step 3. Manual inspection of the difference spectrum (subtraction of matching MS² spectra regardless of precursor ion mass or product ion threshold) was used to confirm a high degree of similarity between matching MS² spectra. Library searching was performed using XCALIBUR (ver. 1.2) in conjunction with NIST MS Search (ver. 1.7). A simple similarity search was selected with the default NIST MS Search options.

Following are examples that illustrate procedures for practicing the invention. These examples should not be construed as limiting. All percentages are by weight and all solvent mixture proportions are by volume unless otherwise noted.

### Example 1

Multi-Pressure Capillary Liquid Chromatography Apparatus with Integrated Electrospray Emitters and its Operation

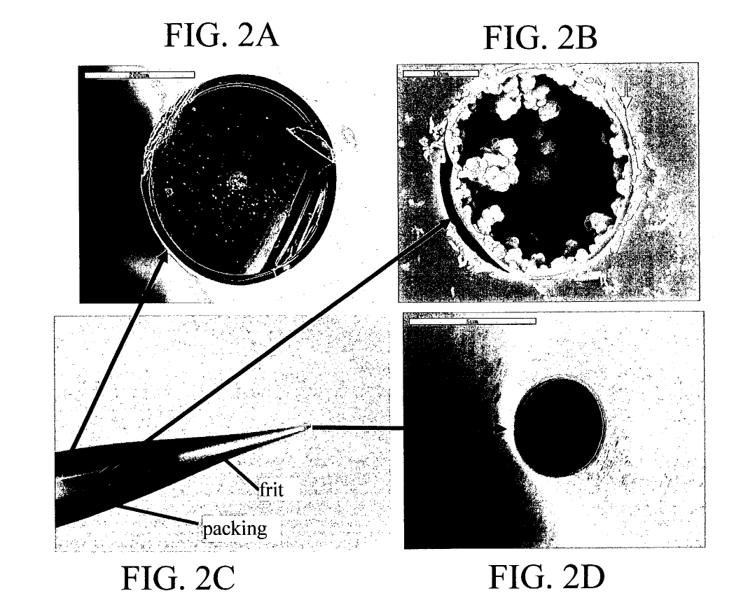

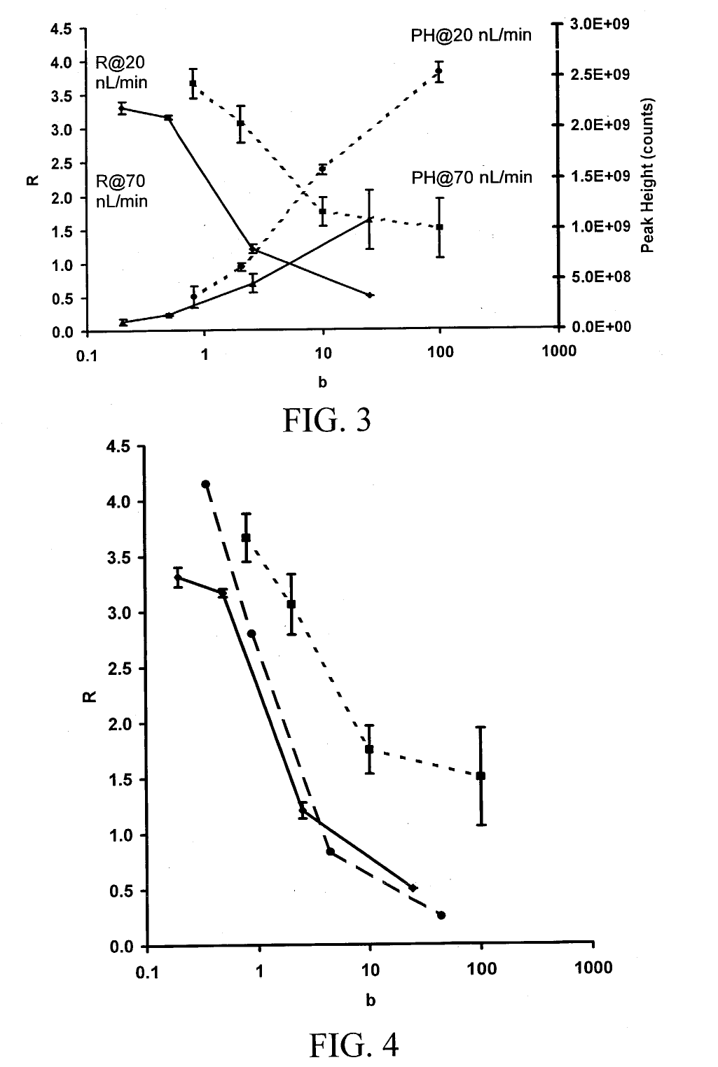

Previous work for high-sensitivity detection of neuropeptides in dialysates utilized 50-μm i.d. integrated electrospray emitters operated at flow rates of 200-800 nL/min. (Emmett, M. R. et al., J. Neurosci. Methods, 1995, 62:141-147; Andren, P. E. et al., Brain Res., 1999, 845:123-129). As described below, reducing the column/emitter dimensions and the separation/electrospray flow rate contributed to improved separation efficiency, mass sensitivity, and concentration sensitivity. Columns (25-μm i.d.) packed with 5-μm particles and 3-μm i.d. electrospray emitters were used to gain substantial miniaturization but retained routine preparation and operation. FIGS. 2A-2C show a column with integrated electrospray emitter (FIG. 2A), a macroporous frit (FIG. 2B), and the orifice at the electrospray emitter tip (FIG. 3C). The emitters could be reproducibly made with a 3.1±0.2-μm i.d. (n=5). Frits were prepared by in situ photopolymerization (Viklund, C. et al., Chem. Mater., 1997, 9:463-467; Chen, J. R. et al., Anal. Chem., 2000, 72(6):1224-1227), instead of sintering particles (Kennedy, R. T. et al., Anal. Chem., 1989, 61:1128-1135), because this procedure allowed the frits to be readily placed near the end of the column to minimize extracolumn band broadening. (Only a 2% loss in resolution was observed for frits placed ˜1 cm from the electrospray emitter orifice in 25-μm i.d. columns operated at separation flow rates 20 nL/min.). In addition, such frits were found to be more reliable than sintered particle frits in that they eliminated the problem of unsintered particles escaping the frit and clogging the electrospray emitter. Because of the small size of the tips, after ˜25 injections of high-volume (1.8 μL) aCSF solutions, clogging of the electrospray emitter by salt deposits caused a decrease in flow rate. Preparing columns daily circumvented clogging problems.

In addition to the column/tip dimensions, a second important feature was an automated two-pressure LC system, which greatly reduced analysis time. This system enabled use of high-flow rates to minimize the sample loading time and column rinse time. Thus, 1.8-μL samples could be loaded in 5 minutes at 370 nL/min. (3200 psi). By switching to a second pump that was already equilibrated to a lower pressure after loading, the separation could be performed at a lower flow rate (20 nL/min at 150 psi or 10 nL/min at 100 psi). Although a single pump could be used to perform these operations, the long time required to stabilize the pressure between flow rate changes would significantly increase the total analysis time.

The operation of the multi-pressure apparatus of the present invention is provided in Table 1.

Table 1. Steps in the operation of the multi-pressure CLC-MS² system of the invention are shown during analysis (e.g., on-line microdialysis) and tuning (off-line, loop injection of a standard).

| | Valve 1 | Valve 2 | | | XYZ | | | |
|---|---|---|---|---|---|---|---|---|
| Analysis Steps | Position | Position | S1 | S2 | Position | N₂(g) | HV | QIT |
| 0.Sample loading | B | B | Open | Closed | 7 mm | Off | Off | Off |
| | | | | | below | | | |
| | | | | | QIT | | | |
| 1.Preconcentrate | A | A | Open | Closed | 7 mm | Off | Off | Off |
| | | | | | below | | | |
| | | | | | QIT | | | |
| 2.Desalt | A | B | Open | Closed | 7 mm | Off | Off | Off |
| | | | | | below | | | |
| | | | | | QIT | | | |
| 3.Trigger gradient | B | B | Open | Closed | 7 mm | Off | Off | Off |
| pumps | | | | | below | | | |
| | | | | | QIT | | | |
| 4.Trigger XYZ | B | B | Open | Closed | at QIT | Off | Off | Off |
| positioner | | | | | | | | |
| 5.Trigger N₂(g) & QIT | B | B | Open | Closed | at QIT | 10 s | Off | On |
| | | | | | | pulse | | |
| 6.Separate/electrospray | B | B | Open | Closed | at QIT | Off | On | On |
| 7.Re-equilibrate | B | B | Open | Closed | 7 mm | Off | Off | Off |
| | | | | | below | | | |
| | | | | | QIT | | | |
| | Valve 1 | Valve 2 | | | XYZ | | | |
| Tuning Steps | Position | Position | S1 | S2 | Position | N₂(g) | HV | QIT |
| 0.Sample loading | B | B | Closed | Open | 7 mm | Off | Off | Off |
| | | | | | below | | | |
| | | | | | QIT | | | |
| 1.Iinfuse tuning | B | A | Closed | Open | 7 mm | Off | Off | Off |
| solution | | | | | below | | | |
| | | | | | QIT | | | |
| 2.Trigger gradient | B | A | Closed | Open | 7 mm | Off | Off | Off |
| pumps | | | | | below | | | |
| | | | | | QIT | | | |
| 3.Trigger XYZ | B | A | Closed | Open | at QIT | Off | Off | Off |
| positioner | | | | | | | | |
| 4.Trigger N₂(g) & QIT | B | A | Closed | Open | at QIT | 10 s | Off | On |
| | | | | | | pulse | | |
| 5.Tune/electrospray | B | A | Closed | Open | at QIT | Off | On | On |

To achieve rapid switching between high-flow rates desired for sample preconcentration and desalting and low-flow rates desired for separation, ionization, and ion transfer efficiency, a two-pressure system was used, as shown in FIGS. 1A-1E. As used herein, position “A” also refers to the first position, and position “B” also refers to the second position. The system utilized two six-port valves (C2 valves, Valco, Houston, Tex.): valve 1 was used to select the pump for separation/electrospray or sample preconcentration/desalting, and valve 2 was the injection valve. To perform an injection, samples were loaded onto the sample loop of the injector valve (valve 2) either by a conventional syringe port or from the microdialysis probe, as shown in FIG. 1A. After the sample loop was filled, the splitter at the waste port (W) was closed and valve 2 actuated to allow sample to be preconcentrated onto the column. The loading pump (model DSFH-151, HASKEL, Burbank, Calif.) was selected by valve 1 (flow path shown in FIG. 1B) and sample pumped onto the column at ˜370 nL/min. After the sample was preconcentrated, valve 2 was actuated to remove the sample loop from the flow path and the column desalted for 5 minutes at 370 nL/min. to desalt the column, as shown in FIG. 1C. Valve 1 was then used to select the gradient syringe pump (model 100DM, Isco, Lincoln, Nebr.), and gradient elution was initiated with a postvalve split from 4 μL/min. to 20 nL/min. unless stated otherwise. Splitter 1 was open and splitter 2 (FIG. 1D) was closed during separation to minimize the dwell time of the gradient. Mobile phase A contained 1% acetic acid in water, and mobile phase B contained 1% acetic acid in methanol. The gradients were changed as described in the text. The solvent reservoir of the sample-loading pump contained 1% acetic acid in water.

A personal computer (model E-4200, Gateway, North Sioux City, S. Dak.) and I/O board (AT-M10-16XE-50, NATIONAL INSTRUMENTS, Austin, Tex.) was used to control the timing of the system via a LABVIEW (NATIONAL INSTRUMENTS) program written in-house. The program independently controlled the injection time, column rinse time, separation/electrospray time, and reequilibration time. All measurements were made with the following CLC parameters, unless specified otherwise: preconcentration time 5 minutes (1.8 μL), desalting time 5 minutes (1.8 μL), separation/electrospray time 15 minutes, and reequilibration time 5 minutes. In addition to valve control, the program triggered a pulse of N₂ gas to remove salt droplets from the electrospray emitter before the emitter was positioned at the entrance of the mass spectrometer. The pulse of N₂ prevented salt contamination of the ion optics by aCSF solutions that accumulated on the tip of the column during sample loading. The tip of the column was automatically positioned at the inlet of the mass spectrometer by a servomotor-driven translation stage and motion controller/driver after injection and rinsing (CMA 12CCCL and ESP 300, Newport). Once the emitter was in position, the gradient syringe pumps and the QIT (LCQ-Deca, THERMOFINNIGAN, San Jose, Calif.) were triggered to begin separation and clectrospray. Electrospray voltage (1.5 kV) was applied at a liquid junction downstream of the gradient splitter tee to prevent contamination by oxidant products. PEEK spacers were constructed in-house to electrically insulate the valves from the actuators (VALCO).

All measurements were made with the following QIT parameters, unless specified otherwise: automatic gain control (AGC) on, maximum AGC time=300 msec, q=0.25, isolation width 3 m/z, normalized collision energy 35%, activation time 0.25 msec, and the default number of microscans and target count values. SEQUEST software was used to correlate uninterpreted MS² data with protein databases for automated peptide sequencing and protein precursor identification. Automatic tuning of the QIT ion optics was accomplished by loop injection of an 18 nM solution of neurotensin 1-11 (qLYENKPRRPYIL, where q=pyroglutamate) in 50% methanol/1% acetic acid after manual positioning of the emitter at <0.5 mm from the entrance to the heated capillary (150° C.). A video microscope (VZM 1000, EDMUND SCIENTIFIC, Barrington, N.J.) was used to monitor the position of the emitter and electrospray stability at 800× magnification. Once tuning was completed, excess neurotensin 1-11 was eluted from the column by ramping the gradient from 50% to 90% B. Splitter 1 was closed and splitter 2 (S1 and S2, respectively, as shown in FIG. 1E) was open during tuning to prevent splitting the sample while maintaining a flow rate equivalent to the separation flow rate.

### Example 2

Gradient Steepness Parameter

To achieve good separation and sensitivity, it was necessary to optimize the gradient steepness parameter (b), which is given by Equation (1)

b=ΔφSV₀t_{G}F=1/1.15 k* (1)

where Δφ is the volume fraction change in organic content during the gradient, S is the solvent strength parameter (the slope of a plot of k′ vs. φ), V₀ is the column dead volume, t_{G} is the gradient ramp time, F is the volumetric flow rate, and k* is the average capacity factor (Snyder, L. R. et al., Anal. Chem., 1988, 55:1412A-1430A). It has previously been observed that an increase in b will decrease resolution and increase sensitivity (Snyder, L. R. et al., J. Chromatogr., 1979, 165:3-30).

To investigate these competing effects for the current system, the resolution and sensitivity for Met-enkephalin and Leu-enkephalin were evaluated as a function of b at 70 nL/min as shown in FIG. 3. The data illustrate the compromise that must be made for sensitivity and resolution with gradient elution. The data also show, however, that decreasing the flow rate to 20 nL/min improved sensitivity and resolution at a given b. (To maintain similar b at lower flow rate, the tG must be increased as shown in Equation 1). This improvement may be explained by a combination of effects. The resolution is improved by higher efficiency at lower flow rates. The sensitivity is improved by the increase in ionization and ion-transfer efficiency that is expected with lower flow rates for electrospray ionization (Wilm, M. et al., Anal. Chem., 1996, 68:1-8). These data illustrate the significant advantage of using low-flow rates for LC-MS with an ESI. Such low-flow rates can be utilized with reasonable b and analysis time if the t_{G} is appropriate and column inner diameter small. The main compromise that was made to maintain similar b values at lower flow rates is the increase in analysis time. Thus, the separation time increased from 1.3 to 5.3 minutes for b of 2.0 when going from 70 to 20 nL/min.

For all further experiments, b of 2.0 (20 nL/min. flow rate) was used; that b value is considerably higher than the b of 0.2 that is usually considered a good compromise among resolution, sensitivity, and analysis time (Snyder, L. R. et al., J. Chromatogr., 1979, 165:3-30). The use of a higher b was dictated by the need to achieve high sensitivity for the trace level detection of neuropeptides while maintaining good chromatographic resolution of Met-enkephalin and Leu-enkephalin. Previous high-sensitivity work on neuropeptides used step gradients (i.e., very high b values), which maximize sensitivity but allow no chromatographic resolution (Snyder, L. R. et al., J. Chromatogr., 1979, 165:3-30). Furthermore, overly high b values can result in peaks that are too narrow to accurately quantitate with the QIT. At the steepest gradient employed in this work (b=100), the peak width was 0.01 minute, which only allows a single scan to be acquired. Employing extremely steep gradients could be useful for qualitative analysis; however, for quantitative analysis it is preferable to collect 8-10 scans across a chromatographic peak. In addition, data-dependent operation of the QIT requires several seconds to complete some scan functions (e.g., full MS, zoom scan, and full MS²). Therefore, it is desirable to have peaks that are at least a few seconds wide. With the b value and flow rates used here, chromatographic peaks were 0.05 minute (3 or) wide at 0.607 of the peak height (1 σ); that width allowed 8-10 mass spectra to be collected over the width of a peak.

For these experiments, it was desirable to utilize short columns (2 cm) because a short column will allow faster loading of sample and therefore shorter analysis times, for a given pressure limit; however, it was also desirable to maintain chromatographic separation. FIG. 4 shows that, with manipulation of b and flow rate, it is possible to obtain similar resolution on 2- and 10-cm-long columns. Thus, resolution is similar over a wide range of b for a 10-cm-long column operated at 50 nL/min. and a 2-cm-long column operated at 70 nL/min. (FIG. 4). Furthermore, as discussed above, higher resolution can be obtained with lower flow rates (see 20 nL/min. data in FIG. 4). This effect, where resolution depends primarily on b and not L, occurs for molecules with large S values such as proteins (Snyder, L. R. et al., Anal. Chem., 1988, 55:1412A-1430A). These results suggest that, for the peptides used here, S is sufficiently large to achieve a similar independence of L. Therefore, the 2-cm capillary columns do not result in a compromise for separation relative to longer columns.

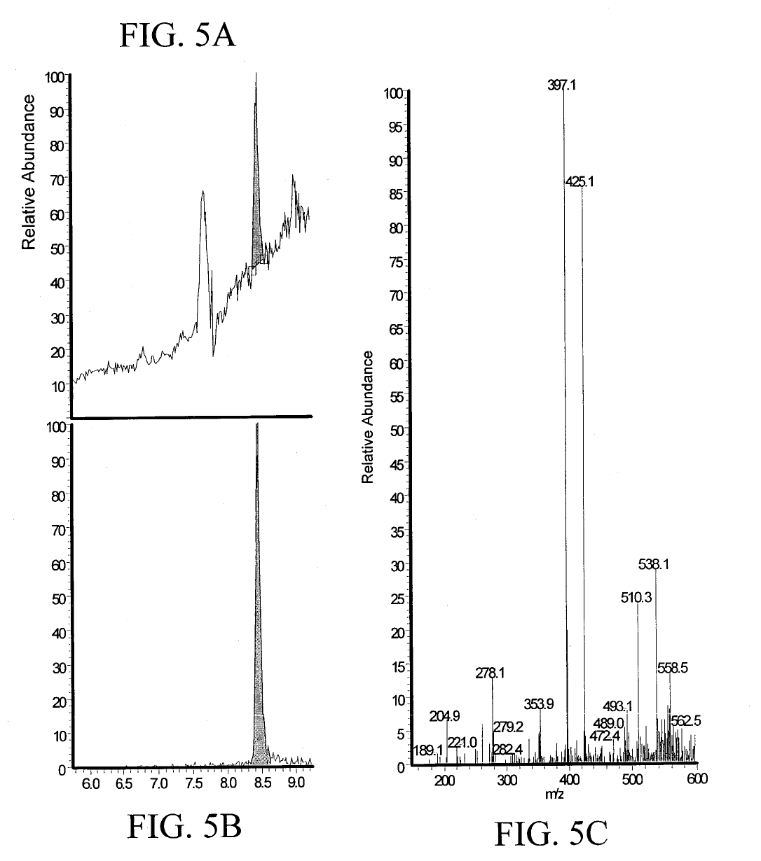

FIGS. 5A-5C illustrate separation with time-segmented MS² detection of 59 amol each (1.8 μL of 33 pM) of Met- and Leu-enkephalin injected on-column. The signal-to-rms noise ration (S/N) in the reconstructed ion chromatogram (RIC) was 65 (FIG. 5B), yielding a mass detection limit, based on the amount that gives a S/N of 3, or 4 amol injected on-column, corresponding to a concentration detection limit of 2 pM for a 1.8-μL injection. Linear calibration curves (R²>0.995) were obtained from 60 pM to 6 nM. RIC peak height relative standard deviations (RSD) were 20% at 60 pM and improved to 5% at 6 nM. Retention time RSD was 2%. Retention time precision was essential because, if a peak elutes outside of a time-segmented scan function, then it will not be detected.

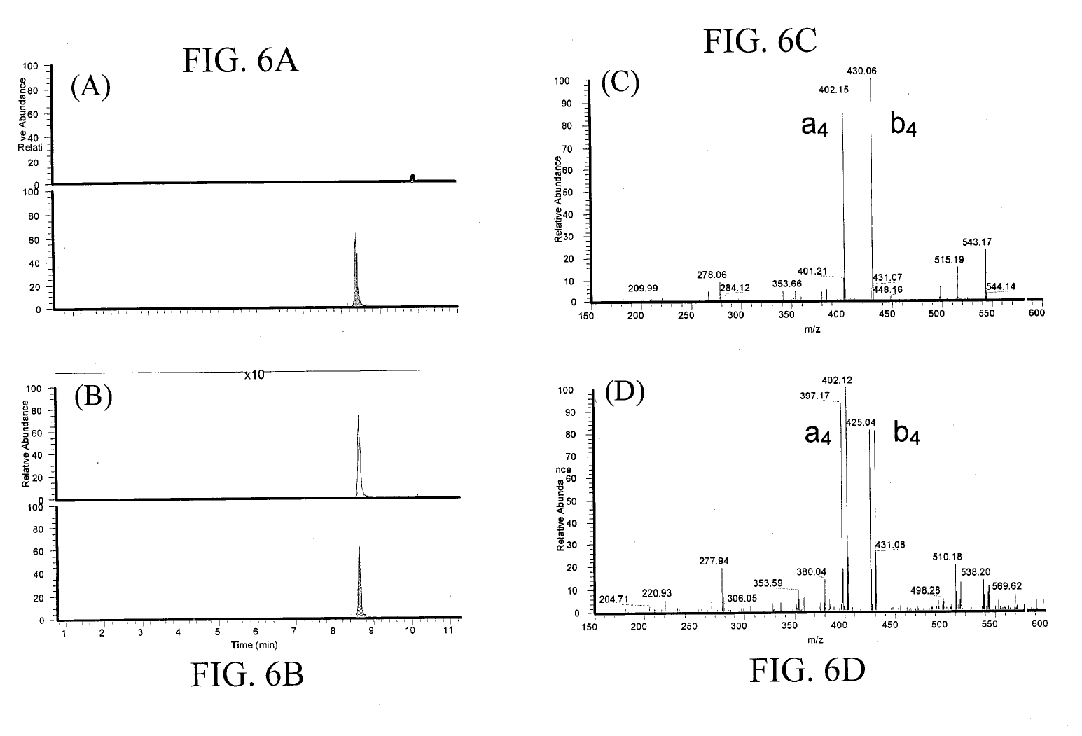

Adsorption of peptides within the injection valve (valve carry-over) can cause false positives. This problem is especially prevalent when injecting high concentrations for calibration prior to high-sensitivity experiments. To avoid this problem, deuterated Leu-enkephalin (YGGF_{D5}L) was used as a calibration standard. The effectiveness of this approach is illustrated in FIGS. 6A-6D, which show a RIC from injection of 600 pM YGGF_{D5}L (FIG. 6A), followed by injection of 60 pM YGGFL (FIG. 6B). (For these experiments, an isolation width of 9 m/z was centered at 558.5 m/z to fragment YGGFL and YGGF_{D5}L simultaneously). The data show that although signal due to carryover for YGGF_{D5}L is obtained in the second chromatogram (bottom panel of FIG. 6B), it does not interfere with the low concentration detection of YGGFL (top panel of FIG. 6B). Moreover, the a₄ and b₄ ions of YGGF_{D5}L at 402 and 430 m/z, respectively, (FIG. 6C) are clearly distinguished from the a₄ and b₄ ions of YGGFL at 397 and 425 m/z (FIG. 6D).

### Example 3

In vivo Monitoring of Known Endogenous Peptides

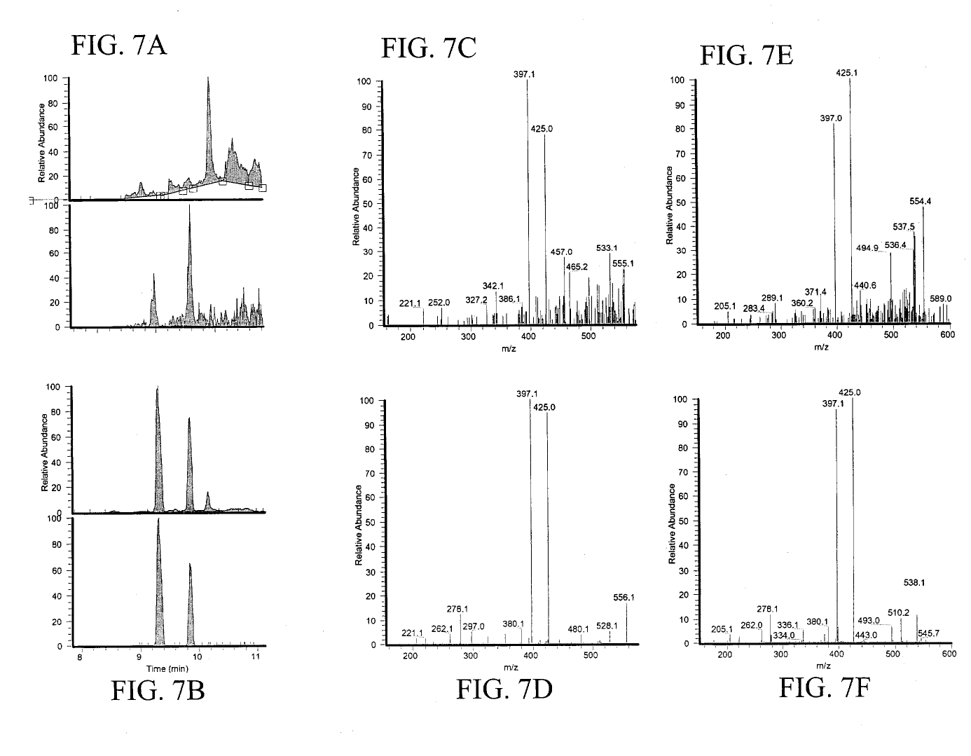

To demonstrate application of the high-sensitivity LC-MS² apparatus and method of the subject invention, dialysate from the rat globus pallidus was analyzed for Met- and Leu-enkephalin. TIC and RIC for Met- and Leu-enkephalin under basal and K⁺-stimulated conditions are shown in FIGS. 7A and 7B, respectively. Under basal conditions, no peaks are discernible for the peptides in the TIC (upper panel of FIG. 7A), although several other peaks are observed. In the time-segmented RIC (lower panel of FIG. 7A), peaks for the target peptides are readily observed because of the reduced background signal in this mode. During stimulation, the signals for the Met- and Leu-enkephalin dominate the RIC and TIC (FIG. 7B). The MS² mass spectrum acquired for Met- and Leu-enkephalin contains all of the expected b- and y-type ions necessary to sequence Met- and Leu-enkephalin at basal levels (FIGS. 7C and E, respectively) and K⁺-stimulated levels (FIGS. 7D and F, respectively). Sequence information, combined with retention times that match those of a standard to within 2%, gives confident identification of the detected peaks as the target peptides.

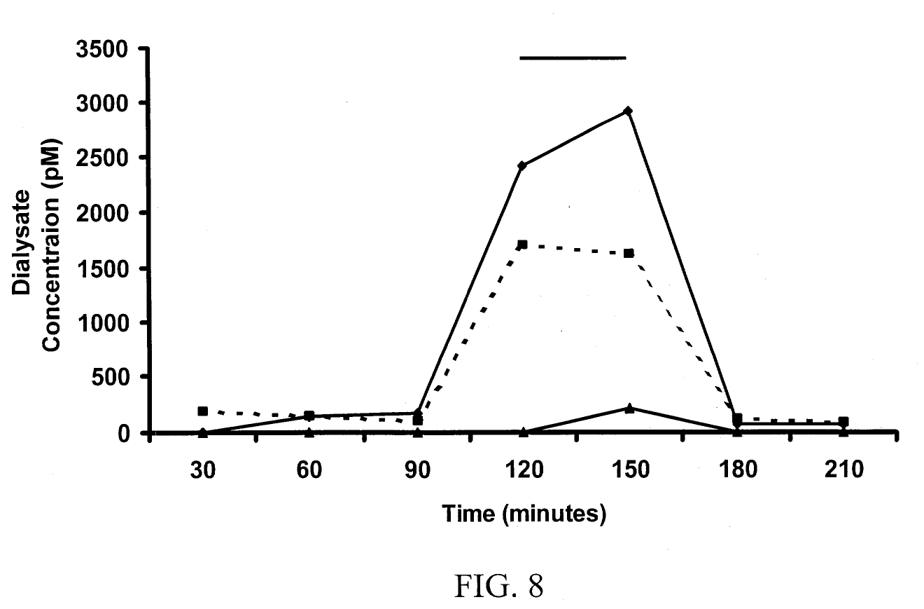

Simultaneous monitoring of Met-enkephalin, Leu-enkephalin, and an unknown peptide during a K⁺ stimulation is illustrated in FIG. 8. For monitoring experiments, 2.0-μL samples were collected on-line every 30 minutes and analyzed (1.8 μL actually injected), resulting in 30-minute temporal resolution. The sensitivity of the system would allow considerable improvements in temporal resolution if dynamic measurements were required. During a 30-minute period, 18 μL of dialysate is generated; however, only 2.0 μL of sample was collected for analysis because the dialysate was pumped through the injection loop to waste while one sample was analyzed by LC-MS². Therefore, the sensitivity of the system is sufficient to achieve at least 3.3-minute temporal resolution (2.0-μL fractions); however, this would require faster on-line analysis or off-line collection and analysis of fractions.

Basal dialysate levels of Met-enkephalin and Leu-enkephalin were 60±30 pM and 70±20 pM, respectively. The maximum K⁺-stimulated levels were 1900±500 pM and 1300±300 pM, respectively (n=7 rats). As reported previously (Maidment, N. T. et al., J. Neuroscience, 1989, 33:549-557), the levels of Met- and Leu-enkephalin are nearly equal even though their precursor protein, preproenkephalin A, contains six repetitions of the Met-enkephalin sequence and only one copy of the Leu-enkephalin sequence. Thus. in vivo processing of preproenkephalin A delivers equal levels to the extracellular fluid despite the presence of more copies of the Met-enkephalin sequence. Previous quantitative work by microdialysis with RIA reported that the total opioid peptide level under basal conditions in an anesthetized rat were ˜150 pM when correcting for an 8% in vitro recovery. These data give a value of 260 pM when correcting for in vitro recovery, indicating reasonable agreement. The slightly higher numbers obtained here could be due to several factors including use of an on-line system that prevented sample loss due to adsorption. In addition, since in vitro recovery does not accurately reflect in vivo recovery, errors could be introduced in such a comparison because of the difference in dialysis conditions. The K⁺-stimulated levels measured for these peptides are 2-5 fold higher than those previously reported (Maidment, N. T. et al., J. Neuroscience, 1989, 33:549-557; Strand, F. L. Neuropeptidies: Regulators of Physiological Processes, MIT Press: Cambridge, Mass. 1999; den Haan, J. M. et al., Science, 1998, 279:1054). The large difference may be due to use of a more aggressive stimulation (30-minute pulse of 150 mM K⁺ versus 2-minute pulse of 100 mM K⁺ in previous work), which may increase the amount released, and to the use of the on-line system. The variability of the measured in vivo levels was greater than that seen for in vitro measurements at the same concentration. This result suggests that a large part of the irreproducibility is due to variables in the in vivo measurement such as probe positioning or inter-animal differences.

### Example 4

In vivo Identification of Unknown Endogenous Peptides

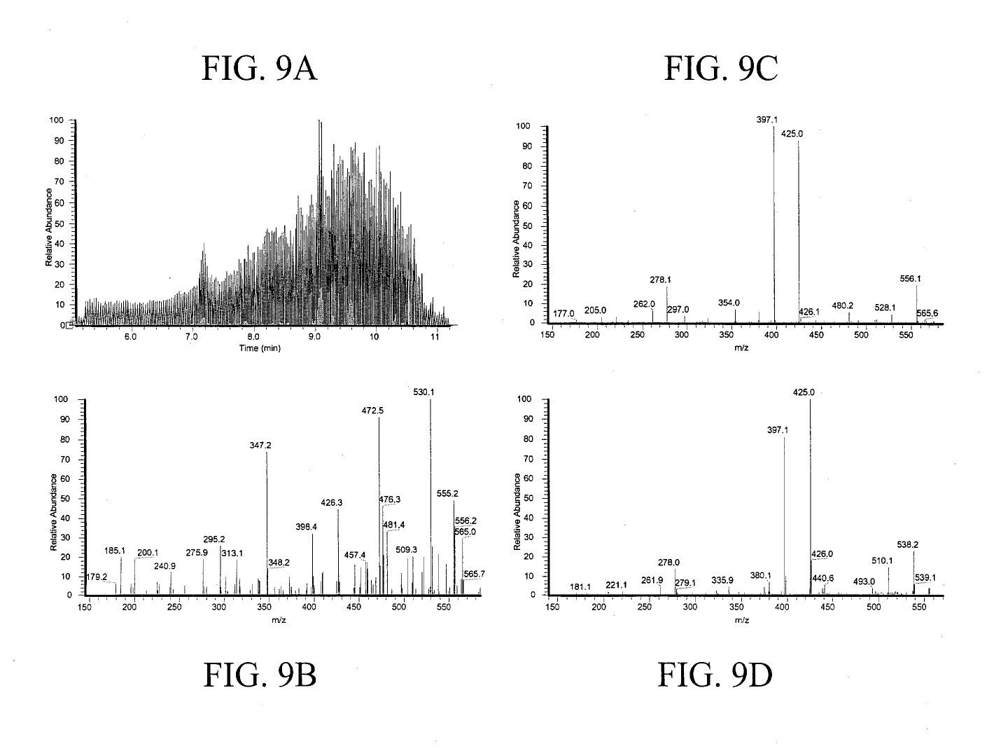

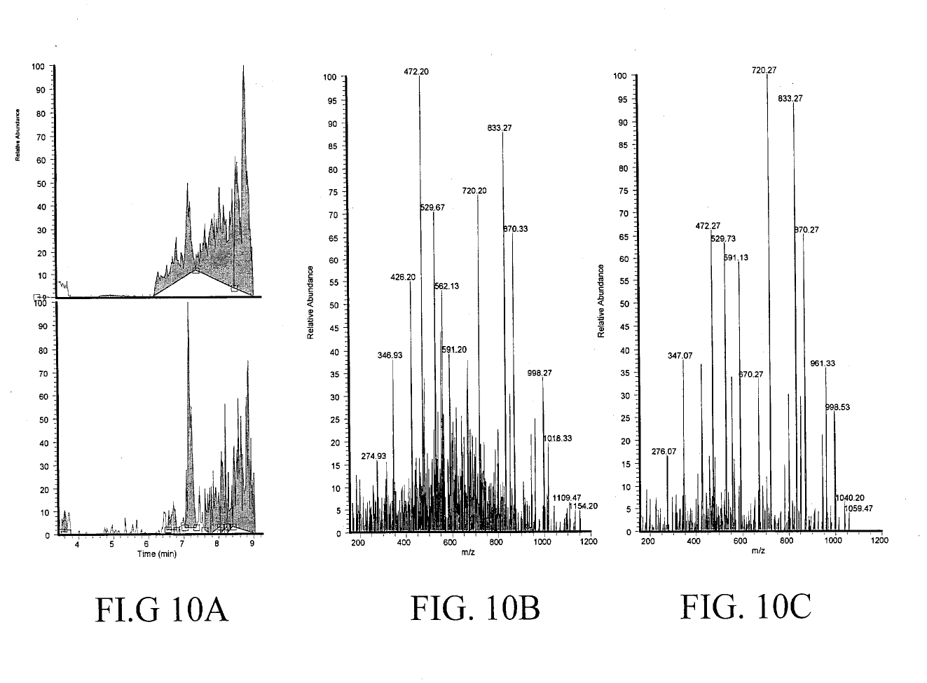

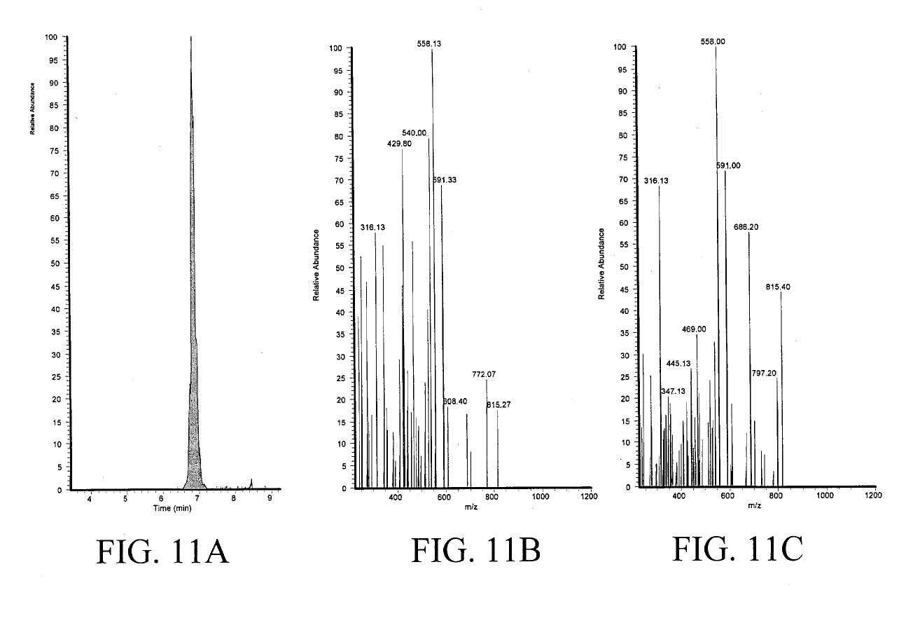

During time-segmented MS² experiments, unknown compounds were observed that were co-released with Met- and Leu-enkephaline. These compounds could be monitored along with the enkephalins, as illustrated by the example in FIG. 8 (see also the peak at 10.1 minutes in the top panel of FIG. 7B). The detection of these compounds even with a narrow isolation width (3 m/z) employed for time-segmented MS² suggested that it might be possible to identify many unknowns collected from the extra-cellular space. To explore this possibility, data-dependent MS² analysis of dialysates was performed. A relatively narrow scan range in the MS² mode (550-600 m/z) was employed during data-dependent MS² experiments in order to maintain high sensitivity. Data-dependent MS² scans of a sample collected during K⁺ stimulation (FIG. 9A) combined with time-segmented MS² data revealed 11 novel peptides. In addition to these unknowns, high-quality MS² spectra for the known peptides Met- and Leu-enkephalin were also obtained in this mode (FIGS. 9C and D, respectively). The data-dependent MS² spectrum for one of the unknown peptides, which eluted at 7.2 minutes, is shown in FIG. 9B. The fragmentation pattern for this unknown suggested a doubly charged peptide ion; however, only a partial sequence was obtained because of the product ion scan range used. Subsequent high-resolution zoom scans confirmed that the unknown peptide was indeed doubly charged (data not shown). Database searching (SEQUEST) identified the peptide as SPQLEDEAKE (SEQ ID NO. 1), a sequence found only within preproenkephalin A. To confirm the identification of this peptide, time-segmented MS² and MS³ scans were performed and compared to synthetic peptide as shown in FIGS. 10 and 11. The retention time (within 2%) and fragmentation pattern (all of the expected b- and y-type ions were observed) were in excellent agreement with the synthetic peptide. In addition, the ion intensities of the standard were within 30% of those for the in vivo sample. Combined, this information gives confident sequence assignment.

The TIC and RIC for the novel peptide in MS² mode (see lower panel of FIG. 10A) shows a surprising amount of chemical noise considering the low background expected for MS² detection. This noise may reflect the complexity of the dialysate samples. However, the S/N is considerably improved for the MS³ TIC (FIG. 11A), suggesting that for some applications this mode of detection may provide improved sensitivity, due to reduced background, over MS² detection despite the loss of signal intensity associated with higher dimensions of mass spectrometry.

From in vivo samples, peptide I₁₋₁₀(SEQ ID NO: 4) was never present above 400 pM (800 amol injected on-column), yet the system was able to identify this peptide from the dialysate. In principle, this approach could be used over a wider scan range to identify more peptides in the dialysate; however, with the weak signals observed (all peptides are at the attomole level), it may be best to perform such characterizations using multiple injections with overlapping, narrow (50 m/z) MS scan ranges. Eleven other peptides were detected and tentatively identified in these data-dependent runs. Further analysis, such as that carried out for peptide I₁₋₁₀, can be utilized to confirm identification of these peptides.

### Example 5

Preproenkephalin A Processing

As indicated above, SPQLEDEAKE (SEQ ID NO: 4) is an amino acid sequence found in preproenkephalin A. Extensive prior studies have not revealed the presence of this peptide as a product of preproenkephalin A processing (Goumon, Y. et al., J. Biol. Chem., 2000, 275:38355-38362; Baird, A. et al., Proc. Soc. Exp. Biol. Med., 1984, 175:304-308; Liston, D. et al., Science, 1984, 225:734-737). Because most work on peptide processing uses antibodies to select peptides with certain epitopes, and because this peptide does not contain any homology with Met- or Leu-enkephalin, it is unlikely that this peptide would have been detected by any conventional processing studies. As depicted in FIG. 12, this peptide is an N-teiminal cleavage product of peptide I (peptide I₁₋₁₀). Peptide I₁₋₁₀ io does not appear to have been produced from peptide I by the cleavage at dibasic sites that is typically associated with endopeptidase activity. A reasonable hypothesis for the production of peptide I₁₋₁₀ is that endopeptidase cleavage of peptide I, a known product of preproenkephalin A in purified bovine chromaffin cells (Stern, A. S. et al., Proc. Natl. Acad. Sci. U.S.A., 1981, 78:1962-1966), yields SPQLEDEAKELQ (SEQ ID NO: 5), which is then cleaved by a carboxypeptidase to yield the SPQLEDEAKE (SEQ ID NO: 4) sequence detected in vivo. This pathway seems likely, given the dibasic site in peptide I and the apparent ubiquity of exopeptidase activity in the brain extracellular space (Zhang, H. et al., J. Mass Spectrom., 1999, 34:377-383).

### Example 6

Discovering Endogenous Neuropeptides by in vivo Microdialysis-CLC-Data-Dependent-MS²

An apparatus of the present invention, as described above in previous Examples, was utilized in these experiments. The system utilizes two six-port valves to select the pump and flow path for preconcentration, desalting and separation/electrospray steps. During the preconcentration and desalting steps the high flow rate pump is selected without splitting of the sample in order to minimize the sample loading time. During the separation/electrospray step the low flow rate pump is selected with splitting of the gradient in order to maximize the separation and electrospray efficiency and to minimize the delay time of the gradient. respectively.

In this work, 8.3 min microdialysis fractions, collected online in a 5 μL sample loop, were analyzed every 35 min by preconcentrating/desalting at 370 nL/min and separating/electrospraying at 10 nL/min. All measurements were made with the following CLC-MS² parameters, unless specified otherwise: preconcentration time=10 min (3.6 μL), desalting time=5 min (1.8 μL), separation/electrospray time=15 min, re-equilibration time=5 min. The mass spectrometer was a quadrupole ion trap (LCQ-Deca, THERMOFINNIGAN, San Jose, Calif.) with the following parameters, unless specified otherwise: automatic gain control (AGC) on, max AGC time=300 msec, q=0.25, isolation width=3 m/z, normalized collision energy=35%, activation time=0.25 msec and the default number of microscans and target count values. Data-dependent MS² spectra were collected in the ‘triple play’ scan mode (MS, zoom, MS²) using precursor ion windows of 550-600 m/z and 500-2000 m/z for 3 and 10 rats, respectively.

Protein identification by shot-gun proteomics is a well-established method with a high success rate. Application of this tool to endogenous peptides in dialysis samples is complicated by: 1) the need to confirm the peptide sequence, not just the protein identity, 2) unknown protease specificity in peptide formation and 3) limited sample availability and low concentrations (Horwitz, W. et al. J. of the Association of Official Analytical Chemists, 1980, 63:1344-1354). Therefore, prior to in vivo experiments, the reproducibility of peptide sequencing and protein precursor identification was explored at the levels expected in these experiments. In addition, it was hypothesized that spectral reproducibility would be important in order to correlate MS² spectra via database and library searching. Unassigned and incorrectly assigned MS² spectra were expected due to non-ideal fragmentation of non-tryptic peptides, product ion signals of low signal-to-noise (S/N) for less abundantly released peptides, and non-peptide contaminants (e.g., plasticizers). Unsuccessful assignment can be a problem even in relatively simple proteomics experiments; for example, in a recent paper, 60% of the MS² spectra observed from a single gel slice remained unassigned following database searching (Simpson, R. J. et al. Electrophoresis, 2000, 21:1707-1732). It was hypothesized that searching a small user library, in addition to a large protein database, would reveal incorrect sequence assignments at attomoles levels. It is well known that mass spectral libraries function well only if good spectral reproducibility is achieved (Yates, J. R. et al. Analytical Chem., 1998, 70:3557-3565; McLafferty, F. W. et al. J. Amer. Soc. for Mass Spectro., 1999, 10:1229-1240; Hough, J. M. et al. Analytical Chem., 2000, 72:2265-2270). Therefore, the present inventors investigated the impact of spectral reproducibility on peptide sequencing and protein precursor identification at attomoles levels. A library of MS² spectra was also constructed in order to find incorrect assignments by the database searching programs.

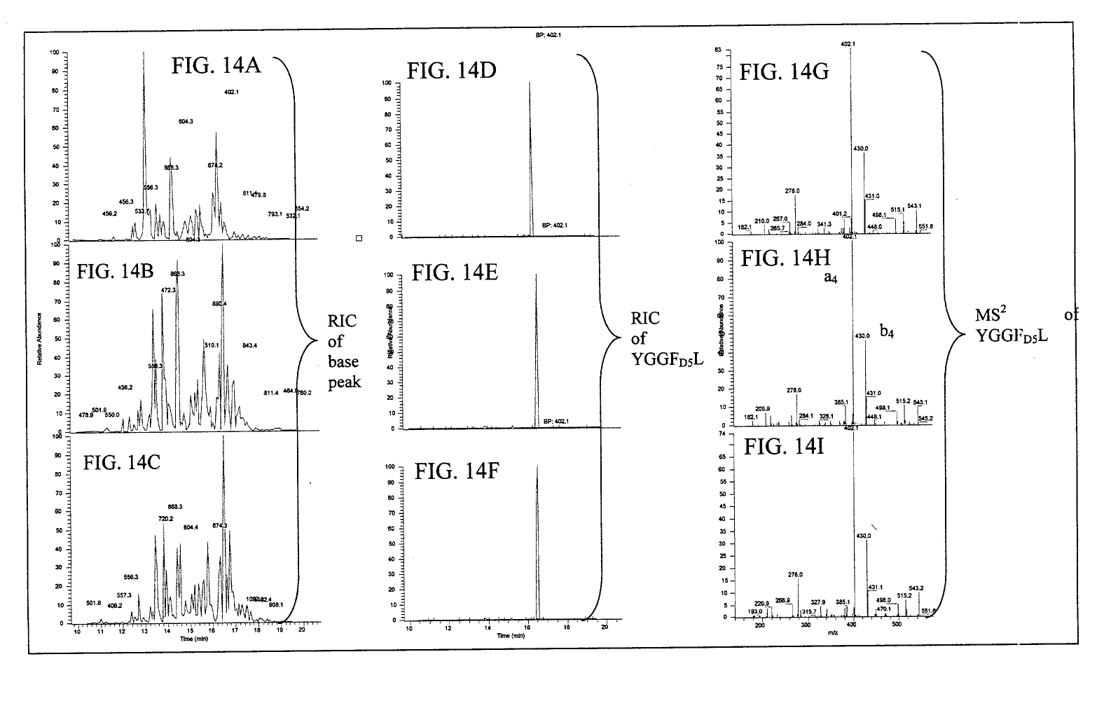

Reproducibility was initially assessed by injecting 0.4 μL of a mixture containing a 10 nM tryptic digest of three proteins (lysozyme, carbonic anyhdrase II, and conalbumin) spiked with 1 nM YGGF_{D5}L in aCSF for CLC-MS² analysis with data-dependent scanning using a 500-2000 m/z precursor ion window. (The concentration of each tryptic peptide is 1 nM). These analyses yielded 132±22 (n=3) data-dependent MS² spectra per chromatogram with a 500-2000 m/z precursor ion window. All errors are reported at the 95% confidence interval for comparison with in vivo results. Examination of the MS² reconstructed ion chromatograms (MS² RIC) in FIGS. 14A-14F showed good reproducibility for the in vitro samples. This observation was supported by subtractive analysis, performed as described in the experimental section, which indicated that 75±18% of the MS² spectra within any two datasets were the same for these in vitro samples. The trace level peptide YGGF_{D5}L (400 amol injected on-column) was observed in every case with good spectral reproducibility (FIGS. 14G-14I).

The results described above indicate the spectral reproducibility that is achieved with this analysis. Determining the reproducibility of peptide sequencing and protein precursor identification was also of interest. Database searching revealed that 62 of the 395 MS² spectra collected (16±7% or 21±6 spectra per sample) were significant (i.e., achieved a SEQUEST ΔCn score greater than 0.1). From these, 40% or 25 MS² spectra (8 were unique) were validated by identifying the same peptide sequence and protein precursor by SEQUEST and Mascot with at least two peptides per protein precursor (see experimental section). Seven of the 8 uniquely validated MS² spectra were observed in all of the samples. All three protein precursors were successfully identified with 3 to 29% sequence coverage. Sixty percent or 37 of the 62 significant MS² spectra were not validated bv a Mascot match. 60% or 37 of 62 were not validated by at least two peptides per protein precursor (i.e., no additional MS² spectra were eliminated by requiring at least two peptides per protein precursor) and 27% or 17 of 62 were redundant (i.e., observed more than once).

The suitability of cle novo sequencing to further validate the MS² spectra was also evaluated. Seventy-five percent (6 of 8 peptide sequences) of the MS² spectra validated by database searching were also validated by de novo sequencing by showing that the 1^{st} ranked de novo-derived partial sequence (Lutefisk program) matched the 1^{st} ranked database-derived sequence better than it matched the 2^{nd} ranked database-derived sequence (Taylor, J. A. and R. S. Johnson Analytical Chem., 2001, 73:2594-2604). In the case of peptide VGDANPALQK from carbonic anhydrase II observed in sample 2, the MS² spectrum yielded a SEQUEST ΔCn equal to 0.31 and a Mascot score indicating homology with greater than 95% probability. The 1^{st} ranked de novo-derived partial sequence from Lutefisk was VGDAN[168.1ILKK, while the 2^{nd} ranked sequences from SEQUEST and MASCOT were GTGKLVALKK and TVAGQVLAKK, respectively.

De novo sequencing was unable to validate database-derived sequences for 2 of the 8 peptides validated by database searching. For example, the peptide FESNFNTQATNR from lysozyme yielded a SEQUEST ΔCn equal to 0.56 and a Mascot score indicating at least homology with 95% probability. The 1^{st} ranked de novo-derived partial sequence from LUTEFISK was [276.1]LAKTNFAM[257.1], while the 2^{nd} ranked sequences from SEQUEST and MASCOT were EFAEYVTNHYR and YEPAQIHLSNTR, respectively. In this case, no sequence homology was observed between the database-derived results and the de novo sequencing-derived results (despite a high quality MS² spectrum with SEQUEST Xcorr=3.7). From these results, it is concluded that validation by de novo sequencing is confirnatory; however lack of a match between de novo sequencing and database searching does not invalidate the sequence identification made by database searching. This is in excellent agreement with results reported previously where the Ist ranked database-derived sequence was found to be more accurate than the 1^{st} ranked de novo-derived sequence (Taylor, J. A. and R. S. Johnson Analytical Chem., 2001, 73:2594-2604).

Previously, the present inventors showed that data-dependent MS² spectra collected using a precursor ion window of 550-600 m/z could be used to provide sequence information for attomole levels of endogenous peptides at picomolar concentrations (Haskins, W. E. et al. Analytical Chem., 2001, 73:5005-5014). While using a narrow precursor ion window (e.g., 50 m/z) during the MS mode of a data-dependent MS² experiment improved selectivity and sensitivity via gas phase fractionation, it was limiting because it allowed detection of a small fraction of the ions observable by the mass spectrometer. Detection of all the observable ions would require multiple overlapping precursor ion windows (Spahr, C. S. et al Proteomics, 2001, 1:93-107; Davis, M. T. etal. Proteomics, 2001, 1:108-117). In this work, the focus was on characterizing the more abundant peptides and protein precursors using a wider precursor ion window than reported previously.

Preliminary experiments on microdialysis samples confirmed earlier observations that 3 peptides (i.e., YGGFM (SEQ ID NO: 1), YGGFL (SEQ ID NO: 7), and SPQLEDEAKE (SEQ ID NO: 4)) could be identified by data-dependent MS² and database searching when using a precursor ion window of 550-600 m/z (animals 1-3). Previous attempts to identify peptides from in vivo microdialysis samples using a 500-2000 m/z window were unsuccessful⁵. In this work, it was found that improving the data-dependent MS² detection limit by increasing the injection volume from 1.8 μL to 3.6 μL and decreasing the separation/electrospray flow rate from 20 nL/min to 10 nL/min enabled collection of many MS² spectra including those for peptides found in the 550-600 m/z range and many novel peptides outside this range.

TABLE 2
Summary of in vivo microdialysis-CLC-MS² results.

| | Spectral | | | Data-Reduction Step | | | | | | | | | | | | | |
|---|---|---|---|---|---|---|---|---|---|---|---|---|---|---|---|---|---|
| | Reproducibility | | | | | | 2 | | | 3 | | 4 | | 5 | | 6 | |
| Animal | | K⁺ₙ_{⁻} | 5% | 1 | | | K⁺ₙ_{⁻} | % | % | | % | | % | | % | | % |
| (n) | Animals | K⁺₍ₙ₊₁₎ | Match | Basal | K⁺ | Total | Basalₙ | Match | Total | No. | Total | No. | Total | No. | Total | No. | Total |
| 1 | 1-2 | 39 | 73 | 149 | 144 | 293 | 51 | 65 | 17 | 34 | 12 | 1, 4, 7, 8 (4) | 1 | 3 | 1 | 4 (1) | 0.3 |
| 2 | 2-3 | 83 | 33 | 128 | 123 | 251 | 56 | 54 | 22 | 22 | 9 | 7, 8 (2) | 1 | 1 | 0 | 0 | 0.0 |
| 3 | 3-4 | 94 | 34 | 148 | 142 | 290 | 49 | 65 | 17 | 20 | 7 | none | 0 | 0 | 0 | 0 | 0.0 |
| 4 | 4-5 | 80 | 16 | 22 | 95 | 117 | 74 | 22 | 63 | 21 | 18 | 4, 6, 8, | 3 | 4 | 3 | 0 | 0.0 |
| | | | | | | | | | | | | 14 (4) | | | | | |
| 5 | 5-6 | 109 | 24 | 138 | 144 | 282 | 85 | 41 | 30 | 28 | 10 | 9, 11, 18, | 4 | 6 | 2 | 9, | 1.4 |
| | | | | | | | | | | | | 21-25, | | | | 11, | |
| | | | | | | | | | | | | 27 (9) | | | | 16, | |
| | | | | | | | | | | | | | | | | 17 | |
| | | | | | | | | | | | | | | | | (4) | |
| 6 | 6-7 | 93 | 29 | 70 | 131 | 201 | 90 | 31 | 45 | 27 | 13 | 10, 11, | 1 | 1 | 0 | 0 | 0.0 |
| | | | | | | | | | | | | 28 (3) | | | | | |
| 7 | 7-8 | 109 | 16 | 96 | 129 | 225 | 93 | 28 | 41 | 25 | 11 | 9, 11, 16, | 4 | 4 | 2 | 0 | 0.0 |
| | | | | | | | | | | | | 17, 20, 25, | | | | | |
| | | | | | | | | | | | | 26, 28, 29 | | | | | |
| | | | | | | | | | | | | (9) | | | | | |
| 8 | 8-9 | 21 | 88 | 151 | 168 | 319 | 32 | 81 | 10 | 24 | 8 | none | 0 | 0 | 0 | 1 (1) | 0.3 |
| 9 | 9-10 | 156 | 10 | 152 | 173 | 325 | 66 | 62 | 20 | 25 | 8 | none | 0 | 0 | 0 | 0 | 0.0 |
| 10 | 10-11 | 48 | 27 | 59 | 66 | 125 | 60 | 9 | 48 | 14 | 11 | 1, 3-9, | 7 | 8 | 6 | 9, 11, | 2.4 |
| | | | | | | | | | | | | 12 (9) | | | | 19 | |
| | | | | | | | | | | | | | | | | (3) | |
| 11 | 11-12 | 128 | 7 | 138 | 137 | 275 | 55 | 60 | 20 | 25 | 9 | 2, 4, 9-16, | 4 | 9 | 3 | 9 (1) | 0.4 |
| | | | | | | | | | | | | 19 (11) | | | | | |
| 12 | 12-13 | 113 | 53 | 172 | 242 | 414 | 63 | 74 | 15 | 26 | 6 | 4 (1) | 0 | 1 | 0 | 0 | 0.0 |
| 13 | 13-1 | 157 | 11 | 155 | 177 | 332 | 85 | 52 | 26 | 31 | 9 | 4, 14, 16 (3) | 1 | 2 | 1 | 0 | 0.0 |
| Total | N/A | 1230 | N/A | 1578 | 1871 | 3449 | 859 | N/A | N/A | 322 | N/A | 55 | N/A | 39 | N/A | 10 | N/A |
| Average | N/A | 95 | 32 | 121 | 144 | 265 | 66 | 50 | 29 | 25 | 10 | 4 | 2 | 3 | 2 | 1 | 0.4 |
| Stand- | N/A | 41 | 25 | 45 | 42 | 83 | 18 | 22 | 16 | 5 | 3 | 4 | 2 | 3 | 2 | 1 | 0.7 |
| ard | | | | | | | | | | | | | | | | | |
| Devi- | | | | | | | | | | | | | | | | | |
| ation | | | | | | | | | | | | | | | | | |
| 95% | N/A | 26 | 15 | 28 | 26 | 51 | 11 | 13 | 10 | 3 | 2 | 3 | 2 | 2 | 1 | 2 | 0.9 |
| Confi- | | | | | | | | | | | | | | | | | |
| dence | | | | | | | | | | | | | | | | | |
| 1 | 2 | 3 | 4 | 5 | 6 | 7 | 8 | 9 | 10 | 11 | 12 | 13 | 14 | 15 | 16 | 17 | 18 |

Columns 1-20 are described from left to right. (1) The animal number. (2-4) Spectral reproducibility was calculated as described in the experimental section. The % match indicates the reproducibility between any two CLC-MS² datasets (n and n+1). (5-20) The number of MS² spectra remaining and the % of the total number of MS² spectra remaining in each data-reduction step (see experimental section). (13) The peptides (listed by the number assigned to the peptide in Table 3) and the total number of peptides observed in each sample (shown in parenthesis).

TABLE 3
Peptides and proteolytic processing patterns observed in vivo.

| | | | | | | | | | Protein |
|---|---|---|---|---|---|---|---|---|---|
| | | | | | | | | | Precursor |
| | | | | | | | | | (no.peptides |
| Animal | | | | | | | | % aa | |
| | (n) | | | | | | | | sequence |
| Peptide | (no. | | | Position | Obs | Mr | Mr | | coverage, |
| (n) | peptides) | Database-Derived Peptide Sequence | De Novo-Derived Peptide Sequence | (aa) | (m/z) | (expt) | (calc) | Delta | no.animals) |
| 1 | 1, 10 (2) | YGGFM (Met-enkephalin) | [220.1]G[278.1] | 100-104, | 574.2 | 573.2 | 573.2 | −0.1 | PEA |
| | | (SEQ ID NO:1) | | 107-111, | | | | | (8, 28 %, 8) |
| | | | | 136-140, | | | | | |
| | | | | 188-192, | | | | | |
| | | | | 212-216, | | | | | |
| | | | | 263-267 | | | | | |
| 2 | 11 (1) | YPVEP | PYVKP | 118-122 | 603.9 | 602.9 | 603.3 | −0.4 | |
| | | (SEQ ID NO:2) | | | | | | | |
| 3 | 10 (1) | YPVEPEEE | YL[226.1]EDPE | 118-125 | 991.4 | 990.4 | 990.4 | 0.0 | |
| | | (SEQ ID NO:3) | | | | | | | |
| 4 | 1, 4, 10- | SPQLEDEAKE (Peptide I₁₋₁₀) | SPKLEDEAKE | 198-207 | 572.9 | | 1143.8 | 1144.5 | −0.8 |
| | 13 (6) | (SEQ ID NO:4) | | | | | | | |
| 5 | 10 (1) | SPQLEDEAKELQ (Peptide I₁₋₁₂) | [174.0]HNEDEAEGALK | 198-209 | 693.3 | 1384.7 | 1385.7 | −1.0 | |
| | | (SEQ ID NO:5) | | | | | | | |
| 6 | 4, 10 (2) | VGRPEWWMDYQ | FYLDEGWMDYK | 219-229 | 733.9 | 1465.8 | 1465.6 | 0.1 | |
| | | (SEQ ID NO:6) | | | | | | | |
| 7 | 1, 2, 10 | YGGFL (Leu-enkephalin) | none | 232-236 | 555.6 | 554.6 | 555.3 | −0.6 | |
| | (3) | (SEQ ID NO:7) | | | | | | | |
| 8 | 1, 2, 4, 1 | YSKEVPEME | FM[168.1]CF[210.2]E | 252-260 | 556.0 | 1110.1 | 1110.5 | −0.4 | |
| | 0 (4) | (SEQ ID NO:8) | | | | | | | |
| 9 | 5, 7, 10, | RKGPGPGGPGGAGGARGGAGGGPSGD | MGWNVEPYAVKNAGGG[226.1]AD | 53-78 | 687.8 | 2060.4 | 2060.0 | 0.4 | Neurogranin |
| | 11 (4) | (SEQ ID NO:9) | (7, 33%, 7) | | | | | | |
| 10 | 6, 11 (2) | KGPGPGGPGGAGGARGGAGGGP | none | 54-75 | 549.6 | 1645.8 | 1644.8 | 1.0 | |
| | | (SEQ ID NO:10) | | | | | | | |
| 11 | 5-7, 11 | KGPGPGGPGGAGGARGGAGGGPSGD | MENGGGAGGRMDRALGGKGN | 54-78 | 635.6 | 1903.7 | 1903.9 | −0.2 | |
| | (4) | (SEQ ID NO:11) | | | | | | | |
| 12 | 10, 11 | GPGPGGPGGAGGARGGAGGGP | [154.1]GGGG[245.0]KGAGGPGGPGH | 55-75 | 759.4 | 1516.8 | 1516.7 | 0.1 | |
| | (2) | (SEQ ID NO:12) | | | | | | | |
| 13 | 11 (1) | GPGPGGPGGAGGARGGAGGGPS | [154.1]GPGAGVKGAGGPGGPGH | 55-76 | 803.0 | 1604.0 | 1603.8 | 0.2 | |
| | | (SEQ ID NO:13) | | | | | | | |
| 14 | 4, 11, 13 | GPGPGGPGGAGGARGGAGGGPSGD | VEHG[259.1]MAGGPGGPGH | 55-78 | 889.1 | 1776.2 | 1775.8 | 0.4 | |
| | (3) | (SEQ ID NO:14) | | | | | | | |
| 15 | 11 (1) | GPGGPGGAGGARGGAGGGPSGD | [154.1]GGPGGAGKRNAGGG[356.2] | 57-78 | 812.0 | 1622.0 | 1621.7 | 0.3 | |
| | | (SEQ ID NO:15) | | | | | | | |
| 16 | 7, 11, 13 | ADTGTTDEFIEAGGDIR | none | 20-36 | 870.5 | 1739.0 | 1738.8 | 0.2 | Fibrinogen |
| | (3) | (SEQ ID NO:16) | | | | | | | Alpha |
| 17 | 7 (1) | DTGTTSEFIEAGGDIR | none | 21-36 | 834.4 | 1666.8 | 1667.8 | −0.9 | Chain |
| | | (SEQ ID NO:17) | | | | | | | Precursor) |
| 18 | 5 (1) | EFIEAGGDIR | [276.1]LEAGDGLR | 27-36 | 553.4 | 1104.8 | 1105.5 | −0.7 | (5, 5%, 4) |
| | | (SEQ ID NO:18) | | | | | | | |
| 19 | 11 (1) | SPVPDLVPG | S[238.1]VSPVPG | 228-236 | 880.4 | 879.4 | 879.5 | −0.1 | |
| | | (SEQ ID NO:19) | | | | | | | |
| 20 | 7 (1) | SQLQEGPPEWK | none | 240-250 | 649.8 | 1297.5 | 1297.6 | −0.1 | |
| | | (SEQ ID NO:20) | | | | | | | |
| 21 | 5 (1) | LVQTQAATTDSDKVDLSIAR | none | 13-32 | 711.0 | 2130.0 | 2131.1 | −1.1 | Fibrinogen |
| | | (SEQ ID NO:21) | | | | | | | Beta Chain |
| 22 | 5 (1) | TTDSDKVDLSIA (Fibrinopeptide B₁₋₁₃) | [220.1]LMDPGKLS[184.1] | 20-31 | 632.9 | 1263.8 | 1263.6 | 0.1 | Precursor |
| | | (SEQ ID NO:22) | | | | | | | (3, 4%, 1) |
| 23 | 5 (1) | TDSDKVDLSIAR (Fibrinopeptide B₂₋₁₄) | none | 21-32 | 660.3 | 1318.7 | 1318.7 | 0.0 | |
| | | (SEQ ID NO:23) | | | | | | | |
| 24 | 5 (1) | IAQDNEPEKPVADSETKM | none | 526-543 | 668.2 | 2001.5 | 2000.9 | 0.6 | Excitatory |
| | | (SEQ ID NO:24) | | | | | | | Amino |
| 25 | 5, 7 (2) | QDNEPEKPVADSETKM | [171.0]AWGAFHPVVJNPKM | 528-543 | 909.6 | 1817.1 | 1816.8 | 0.3 | Acid |
| | | (SEQ ID NO:25) | | | | | | | Transporter |
| 26 | 7 (1) | DNEPEKPVADSETKM | N[244.0][226.1]PVEANSET[259.1] | 529-543 | 845.4 | 1688.8 | 1688.8 | 0.0 | 1 (4, 3%, 2) |
| | | (SEQ ID NO:26) | | | | | | | |
| 27 | 5 (1) | EPEKPVADSETKM | none | 531-543 | 730.9 | 1459.8 | 1459.7 | 0.1 | |
| | | (SEQ ID NO:27) | | | | | | | |
| 28 | 6, 7 (2) | AKAPAPAAPAAEPQAEAPVAS | none | 191-211 | 957.9 | 1913.7 | 1914.0 | −0.3 | Brain |
| | | (SEQ ID NO:28) | | | | | | | Acidic |
| | | | | | | | | | Membrane |
| | | | | | | | | | Protein |
| | | | | | | | | | (2, 13%, 2) |
| 29 | 7 (1) | AKAPAPAAPAAEPQAEAPVASSEQSVAVKE | none | 191-220 | 958.1 | 2871.2 | 2871.5 | −0.2 | |
| | | (SEQ ID NO:29) | | | | | | | |
| 1 | 2 | 3 | 4 | 5 | 6 | 7 | 8 | 9 | 10 |

Columns 1-10 are described from left to right. (1) The peptide number. (2) The animals (listed by the number assigned to the animal in Table 2) and the total number of animals that each peptide was observed in (shown in parenthesis). (3) Database-derived peptide sequences. (4) De novo-derived peptide sequences where regions of de novo-validated sequences with homology to database-derived sequences are underlined. (5) The amino acid positions of the peptide within the protein precursor. (6) The precursor ion's observed mass-to-charge ratio (m/z). (7) The experimental and (8) calculated monoisotopic mass of the peptide. (9) The difference between the experimental and calculated monoisotopic mass. (10) The protein precursors for 29 peptides in vivo. The number of peptides observed, the % sequence coverage by amino acid count and the total number of animals each peptide was observed in, are shown in parenthesis.

Twenty-six-3.6 μL injections of CSF microdialysate, collected on-line from animals during basal and depolarization conditions, yielded 121±28 (n=13) and 144±26 (n=13) data-dependent MS² spectra per chromatogram, respectively, with a 550-600 m/z (n=3) and a 500-2000 m/z (n=10) precursor ion window. (Similar numbers of validated MS² spectra were obtained with the two precursor ion windows as shown in Table 2; therefore the results are presented together). FIGS. 15-18 are examples of results obtained in vivo.

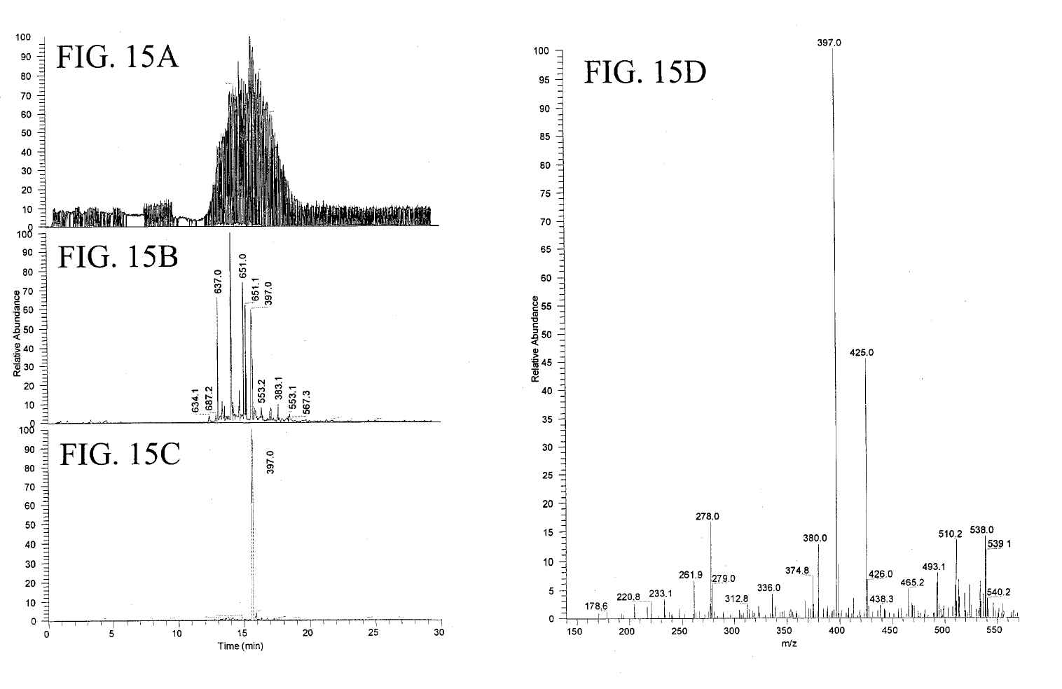

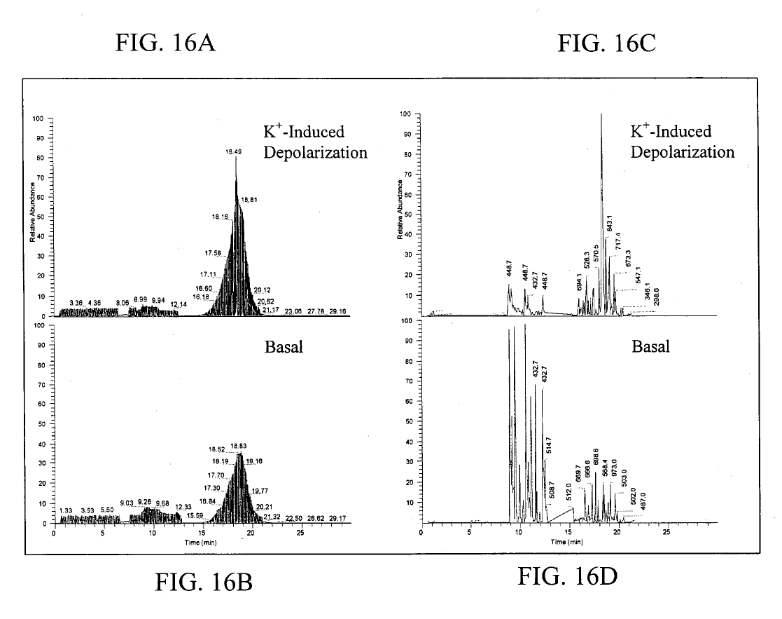

Results for a single animal are presented in FIGS. 15A-15D and FIGS. 16A-16D. FIG. 15A shows the total ion chromatogram (TIC), FIG. 15B shows the MS² RIC of the most abundant product ions, and FIG. 15C shows the RIC of the 556→397+425 transition for YGGFL. FIG. 15D shows the MS² spectrum for YGGFL. As expected, the a₄ and b₄ ions of YGGFL observed in vivo are 5 m/z less than the a₄ and b₄ ions of YGGF_{D5}L observed in vitro (FIG. 14D). Thus, the YGGF_{D5}L standard used to calculate recovery through the microdialysis probe and confirm sensitivity did not interfere with the analysis. FIGS. 16A-16D compares the MS² RIC for the most abundant product ions observed in vivo for basal (FIGS. 16B and 16D) and depolarization (FIGS. 16A and 16C) conditions for a single animal. Clear differences in the chromatograms suggest detection of a many changes in the chemical environment of the brain extracellular space as a result of this manipulation. In this animal, 96 and 129 MS² spectra were collected during basal and depolarization conditions, respectively (Table 2). Subtraction of the two datasets (as described in the experimental section) yielded 93 MS² spectra that were observed only during depolarization. The other MS² spectra were primarily due to contaminants from the microdialysis probe. Database searching revealed that 25 of these MS² spectra were significant. From these, 9 MS² spectra were validated by identifying the same peptide sequence and protein precursor by SEQUEST and MASCOT with at least two peptides per protein precursor. Thus, 9 peptide sequences and 4 protein precursors were observed during depolarization conditions in this animal (Table 3).

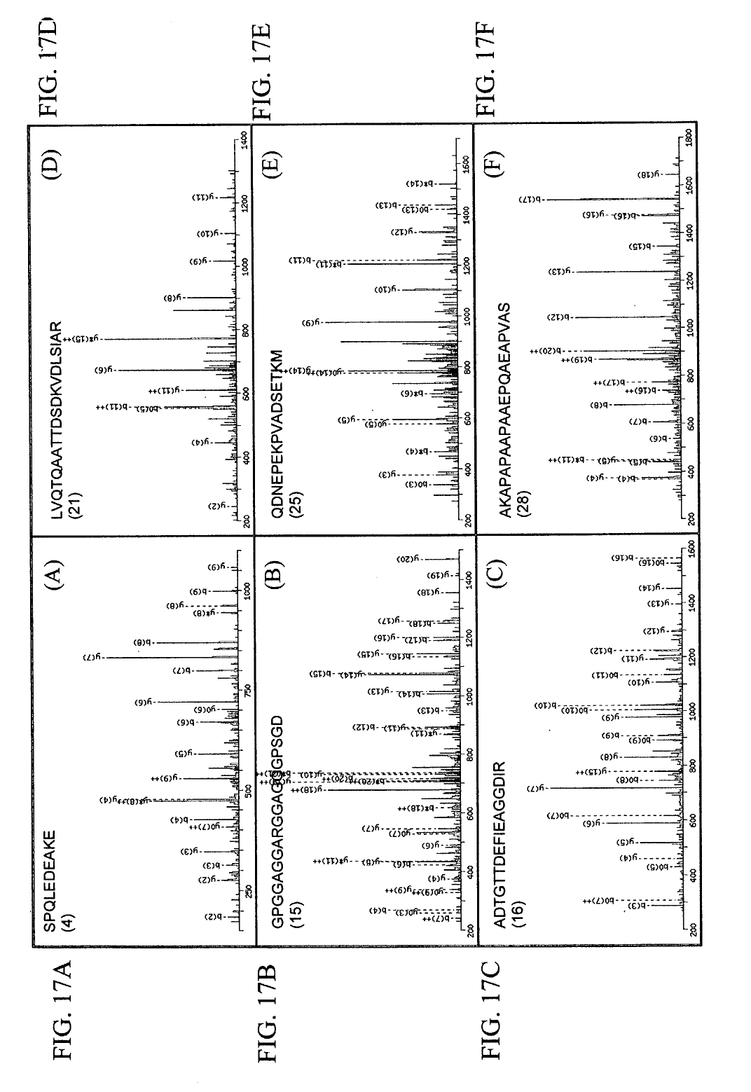

Results for all 13 animals are presented in Tables 2 and 3. Subtractive analysis revealed that 859 of the 3,349 MS² spectra collected (25±10% or 66±11 MS² spectra per animal) were found only during depolarization (Table 2). (Emphasis for data analysis was placed on the spectra observed during depolarization because signaling neuropeptides are expected to be released under these conditions.) Database searching showed that 322 of 3,349 MS² spectra (10±2% or 25±3 MS² spectra per animal) were significant (i.e., achieved a SEQUEST ΔCn score greater than 0.1). From these, 17% or 55 of 322 MS² spectra (29 were unique) were validated by identifying the same peptide sequence and protein precursor by SEQUEST and MASCOT with at least two peptides per protein precursor (see experimental section). In individual samples, anywhere from zero to 9 of the 29 uniquely validated MS² spectra were observed. Six protein precursors were successfully identified with 3 to 28% sequence coverage (Table 3). Eighty-three percent or 267 of the 322 significant MS² spectra (SEQUEST) were not validated by a MASCOT match, 83% or 267 of 322 were not validated by at least two peptides per protein precursor (i.e., no additional MS² spectra were eliminated by requiring at least two peptides per protein precursor) and 17% or 56 of 322 were redundant. Because of redundancy in the animals, 71% or 230 of 322 significant MS² spectra corresponded to unique peptide sequences. Twenty-nine of 230 were uniquely validated and 201 of 230 were unique but not validated. Representative MS² spectra and sequences for the 29 uniquely validated peptides produced from proteolytic processing of PEA, neurogranin, brain acidic membrane protein, fibrinogen α and β and excitatory amino acid transporter are shown in FIGS. 17A-17F to illustrate the quality of data obtained in this work.

Additional validation was obtained for 62% (18 of 29) of the peptides by showing that the 1^{st} ranked LUTEFISK de novo-derived partial sequence matched the 1^{st} ranked database-derived sequence better than it matched the 2^{nd} ranked database-derived sequence (Taylor, J. A. and R. S. Johnson Analytical Chem., 2001, 73:2594-2604). In the case of peptide SPQLEDEAKE (SEQ ID NO: 4) from PEA observed in animal 10, the MS² spectrum yielded a SEQUEST ΔCn equal to 0.16 and a MASCOT score indicating at least homology with 95% probability. The 1^{st} ranked de novo-derived partial sequence from LUTEFISK was SPKLEDEAKE (SEQ ID NO: 4) (Q and K are isobaric in the quadrupole ion trap), while the 2^{nd} ranked sequences from SEQUEST and MASCOT were RRIDEAKE and VNVRSIAGEMGA, respectively. As with the in vitro study, this is in excellent agreement with results reported previously where the 1^{st} ranked database-derived sequence was found to be more accurate than the 1^{st} ranked de novo-derived sequence (Taylor, J. A. and R. S. Johnson Analytical Chem., 2001, 73:2594-2604).

Automated de novo sequencing was able to validate only a fraction (62%) of the database-derived sequences (see Table 2). For the peptide AKAPAPAAPAAEPQAEAPVAS from brain acidic membrane protein, the MS² spectrum yielded a SEQUEST ΔCn equal to 0.44 and a Mascot score indicating at least homology with 95% probability. No quality sequences were found by Lutefisk, while the 2^{nd} ranked sequences from SEQUEST and MASCOT were EGGEAEAPAAEGGKDEAAGGAA and LTRRIGVGVAVLNRLLY, respectively. In this case, no sequence homology was observed between the database-derived results and the de novo sequencing-derived results. The exact reason for this is unknown; however, it is not due to a low S/N MS² spectrum. FIG. 17F shows a high S/N MS² spectrum for this peptide. Clearly, automated de novo sequencing with LUTEFISK is a more stringent criterion for sequence validation than comparing sequences from two different database-searching programs. As with the in vitro study, from these results, it was concluded that validation by automated de novo sequencing is confirmatory; however lack of a match between de novo sequencing and database searching does not invalidate the sequence identification made by database searching.

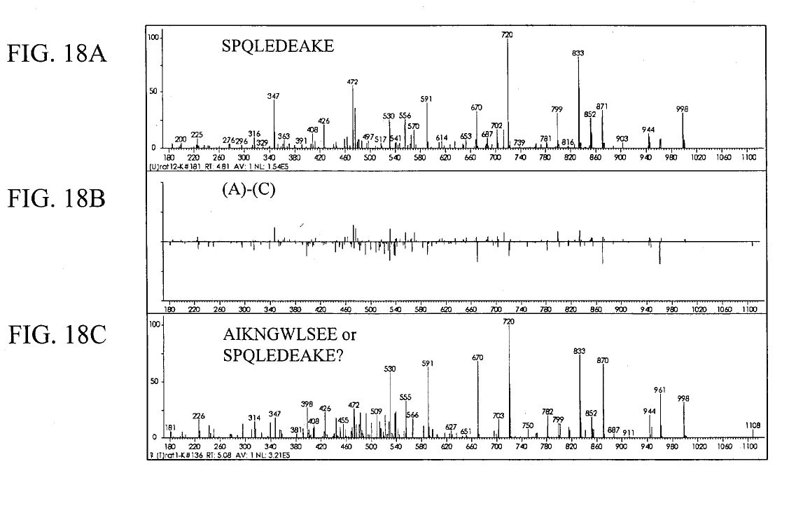

The use of a “library” of spectra collected in vivo was useful in determining if MS² spectra had been incorrectly assigned. Library searching of the 55 MS² spectra validated by database searching against the 322 significant MS² spectra revealed 10 (3%) incorrect assignments by the database searching programs as shown in Table 2. In the case of SPQLEDEAKE (SEQ ID NO: 4) observed in rat 1, the MS² spectrum was incorrectly interpreted for the precursor ion at 574.4 m/z (z=+2) due to a low SIN MS² spectrum (particularly in the 300-600 m/z region) as shown in FIG. 18C. The database-derived sequence was AIKNGWLSEE with a SEQUEST ΔCn equal to 0.11 and a MASCOT score indicating at least homology with 95% probability. The MS² spectrum was successfully correlated with SPQLEDEAKE (SEQ ID NO: 4) by searching the in vivo library for matching MS² spectra as shown by the difference spectrum in FIG. 18B. Library searching was performed using XCALIBUR (ver. 1.2) in conjunction with NIST MS Search (ver. 1.7). A simple similarity search, where the algorithm weights the MS² spectra by mass to find MS² spectra that are similar to the query MS² spectrum, was selected with the default NIST MS² Search options. The MS² spectrum for SPQLEDEAKE from rat 1 (FIG. 18C) matched the significant MS² spectra from the other rats in the in vivo library with less than a 38% difference in the product ion intensities of the MS² spectra. For example, the MS² spectrum for rat 12, with a SEQUEST ΔCn equal to 0.23, is shown in FIG. 18A and the difference spectrum (i.e., FIGS. 18A-18C) is shown in FIG. 18B. Based on the frequency of these observations (i.e., SPQLEDEAKE was observed in 6 of 13 animals while AIKNGWLSEE was observed only once), the SEQUEST ΔCn scores and the MS² spectrum for the synthetic peptide SPQLEDEAKE (Haskins, W. E. et al. Anal. Chem., 2001, 73:5005-5014), the correct assignment for the MS² spectrum in (FIG. 18C) is SPQLEDEAKE rather than AIKNGWLSEE. Co-release of other PEA-derived peptide sequences provides additional evidence in support of this argument. It was found that library searching ‘rescued’ a few incorrectly assigned MS² spectra and thus slightly improved the reproducibility of peptide sequencing and protein precursor identification in vivo.

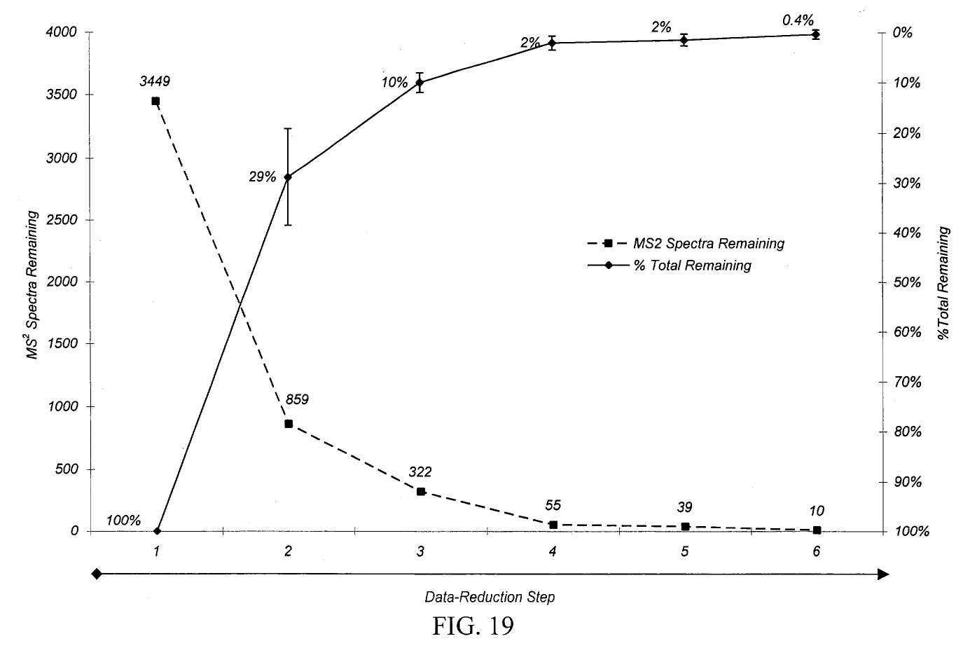

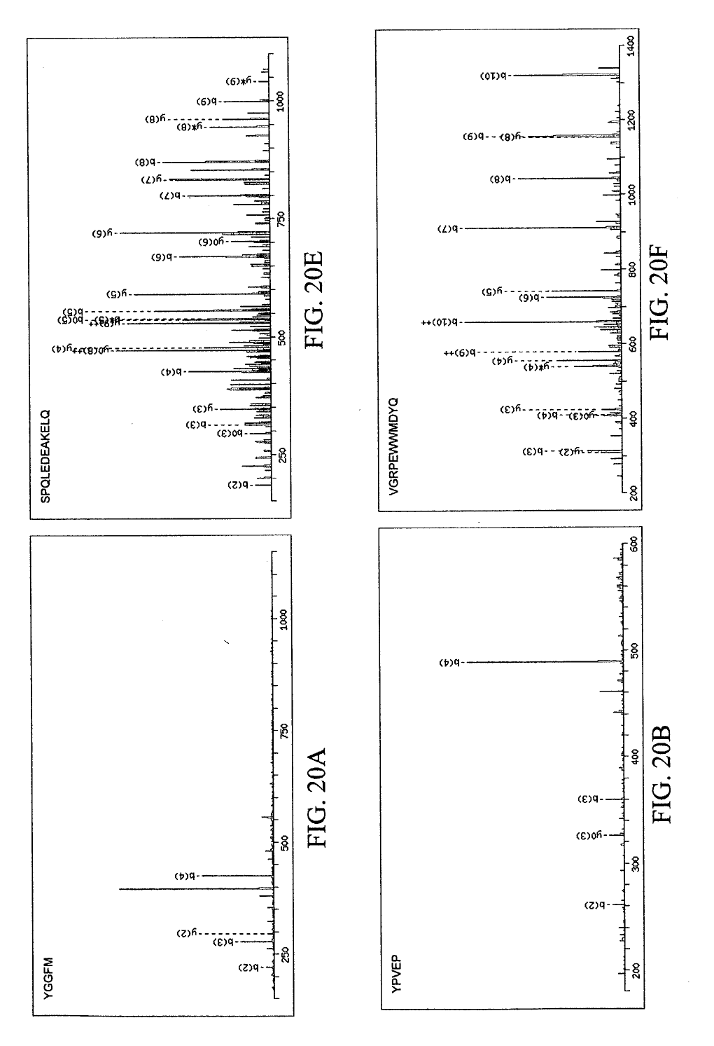

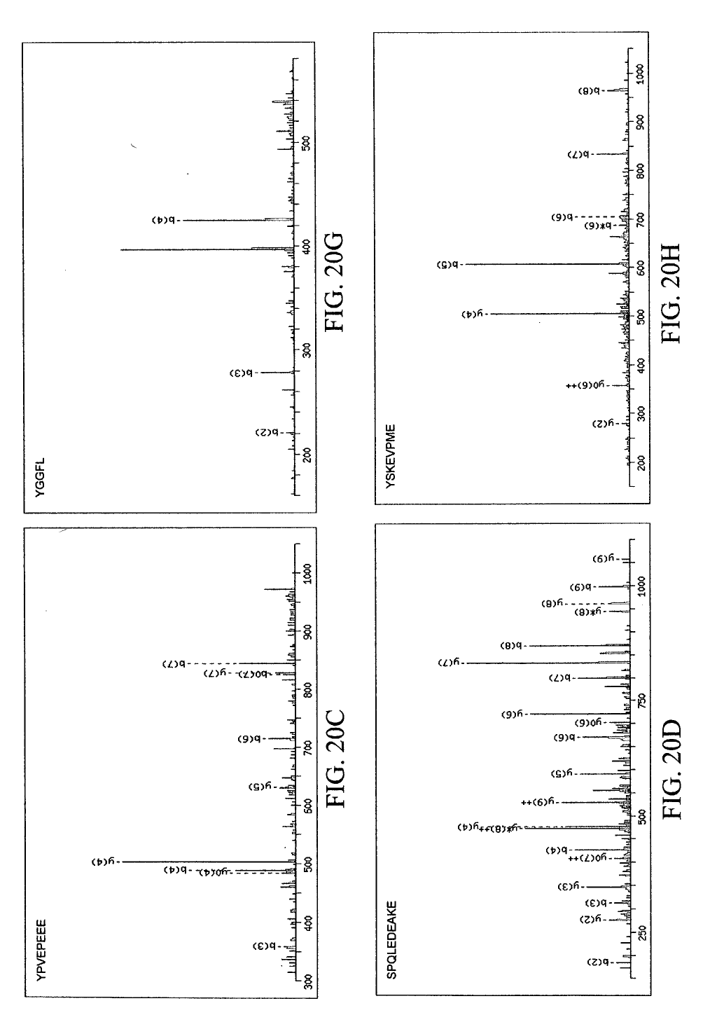

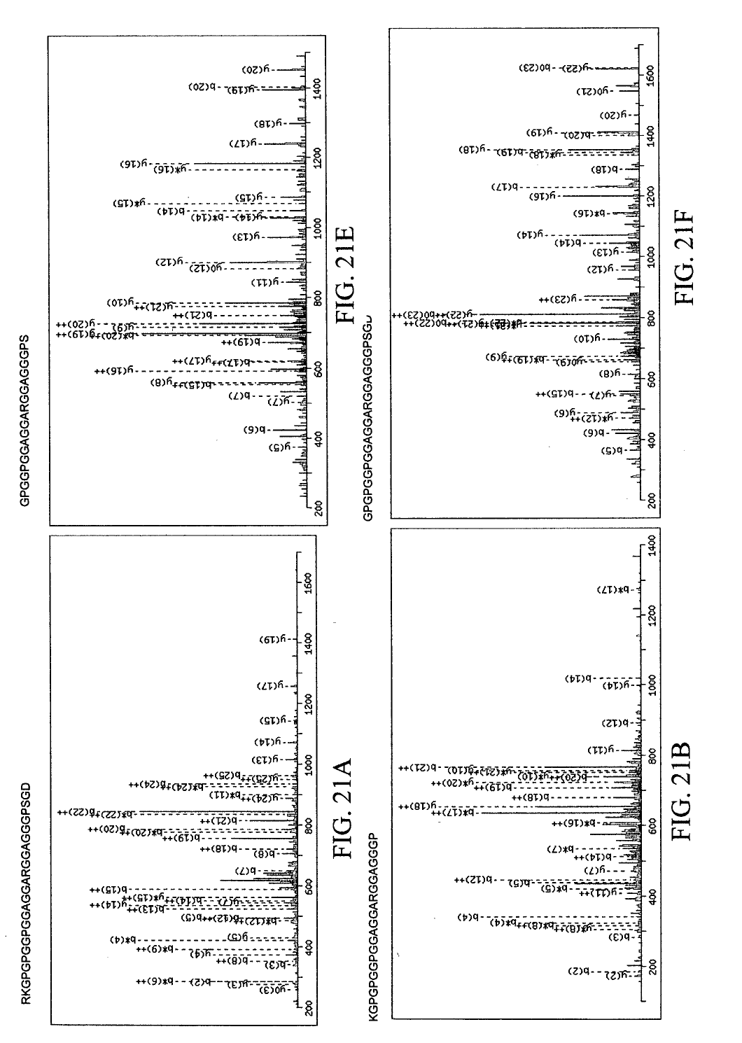

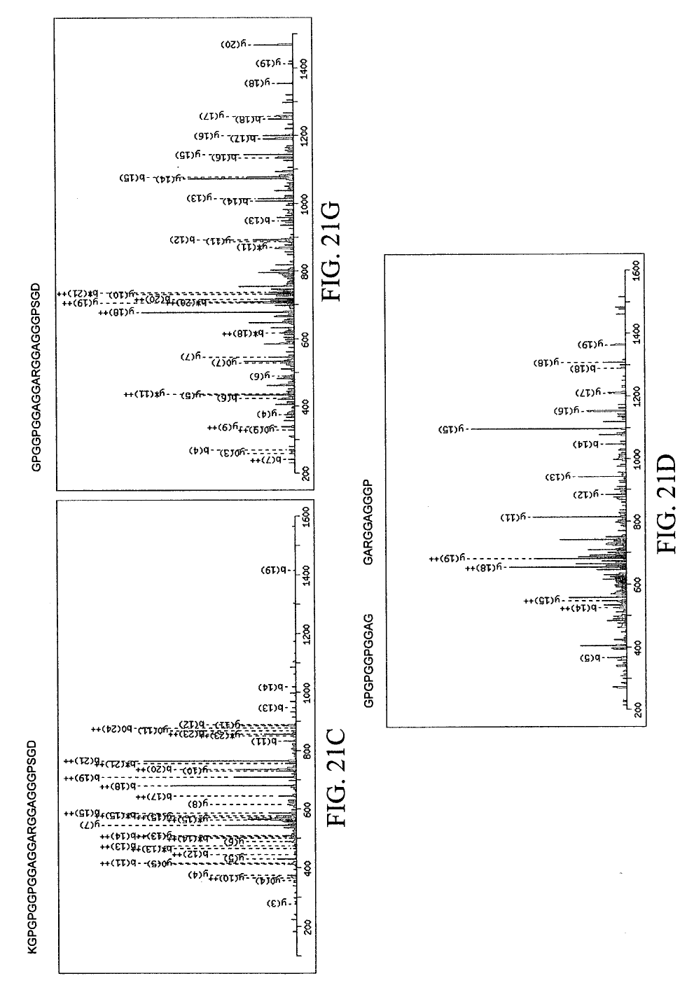

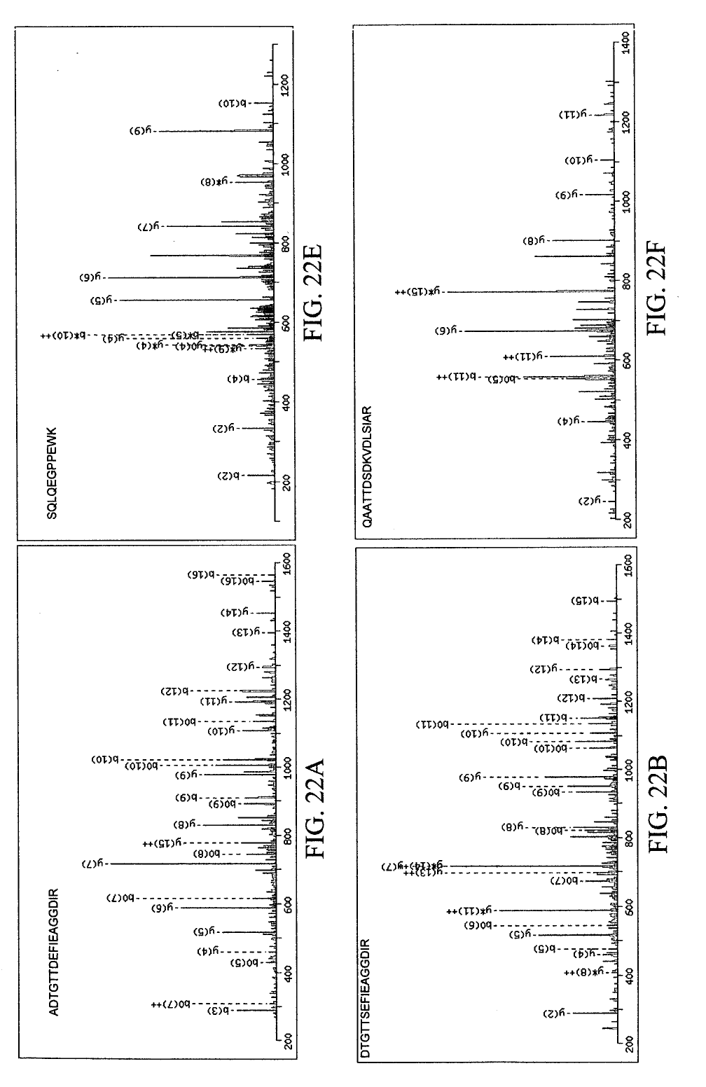

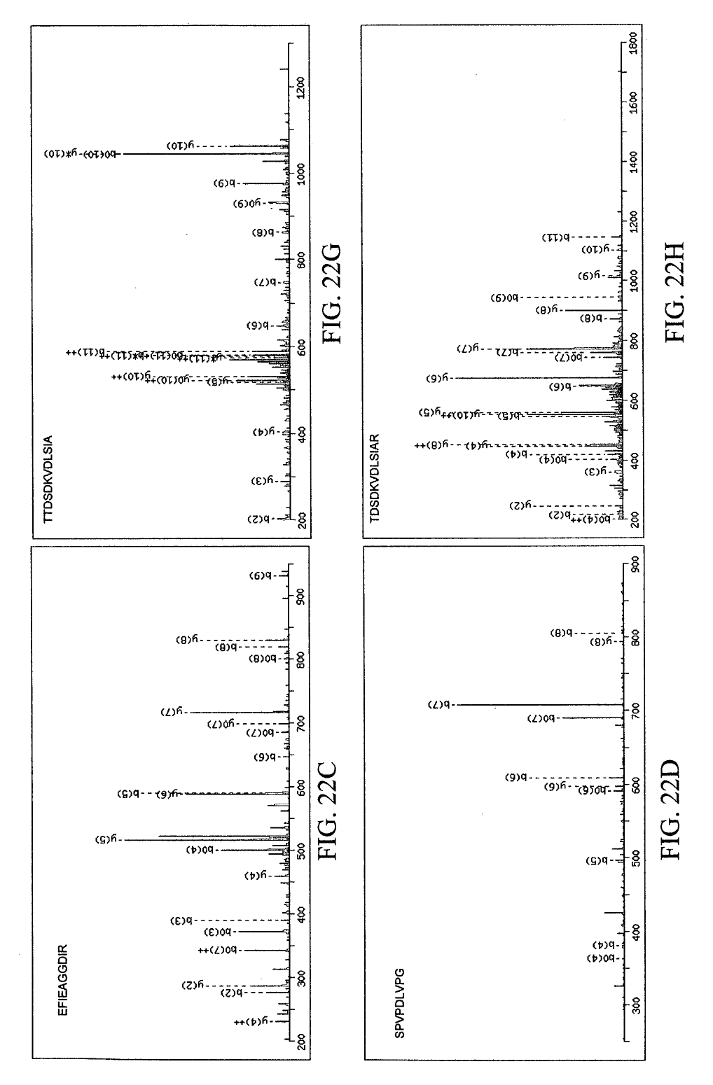

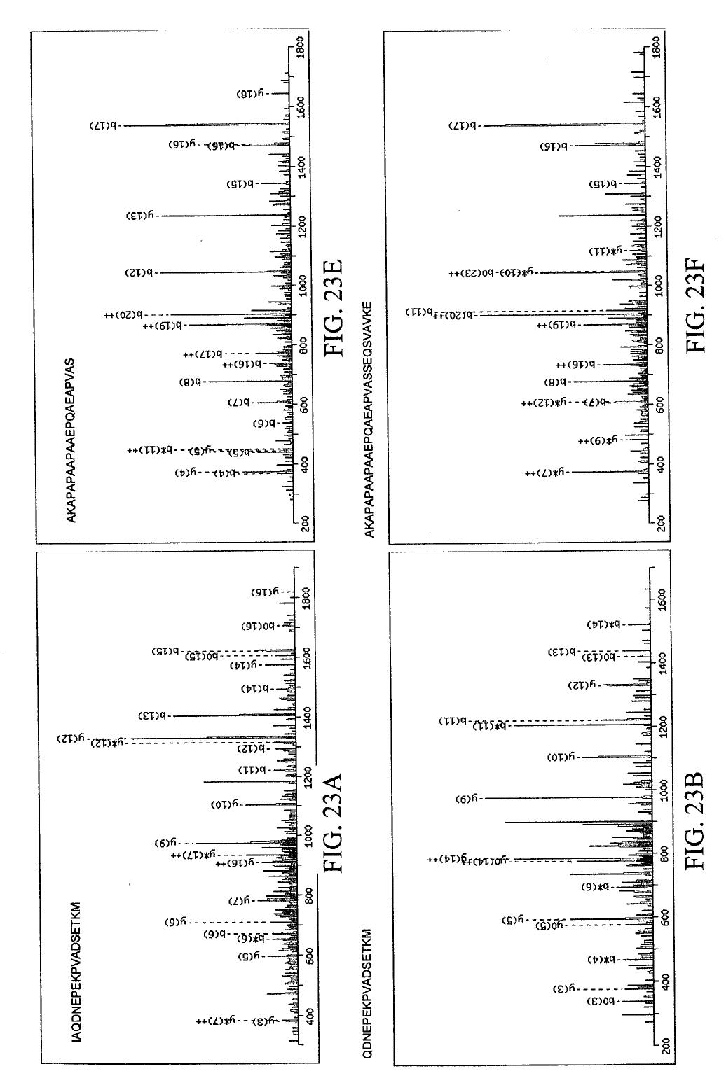

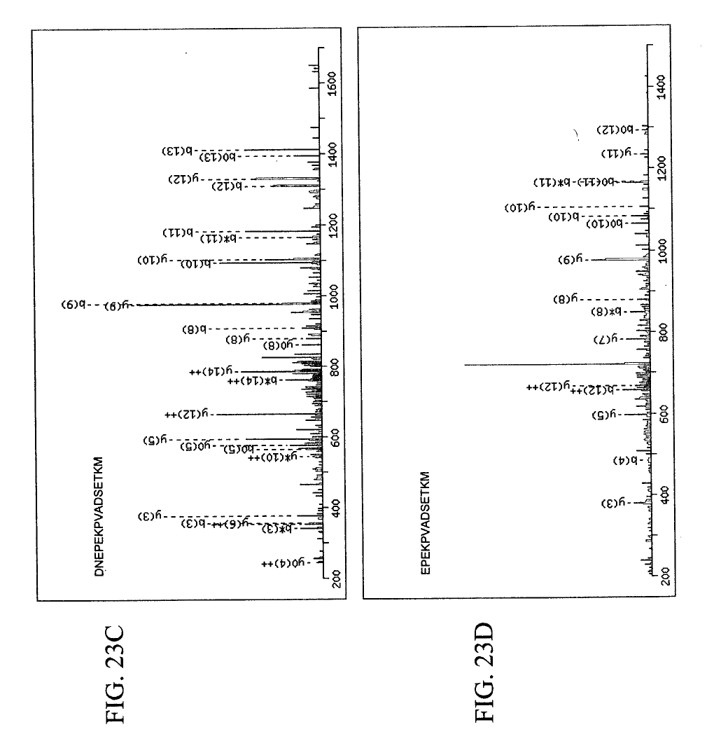

A summary of the in vivo results is shown in FIG. 19 to illustrate the data-reduction strategy described in the experimental section. A decrease in the number of MS² spectra remaining at each data-reduction step clearly indicates successively more stringent criteria for sequence validation. Of the MS² spectra collected in vivo, there were 3,449 MS² spectra remaining following data-reduction step 1, 859 depolarization-specific MS² spectra (29±10%) remaining in step 2, 322 significant MS² spectra (10±2%) remaining in step 3, 55 database-validated MS² spectra (2±1%) remaining from step 4 (29 were unique), 39 de novo-validated MS² spectra (2±1%) remaining in step 5 and 10 incorrectly assigned MS² spectra (0.4±0.9%) remaining in step 6. Subtraction of the 55 validated MS² spectra and 10 incorrectly assigned MS² spectra from the 322 significant MS² spectra yielded 257 MS² spectra that did not meet the criteria for validation. From the remaining 257 MS² spectra, 56 were redundant and 201 were unique (i.e., they did not match other MS² spectra in the library). In order to prevent false-positives, further validation will be required to confirm the corresponding peptide sequences and protein precursors.

No MS² spectra collected during basal conditions could be validated in any of the animals. This suggests that the peptide sequences that were observed during depolarization are not simply the products of neuronal injury caused by the microdialysis probe. This also suggests that with anesthetized animals and the sampling methods used, not enough peptide can be recovered during basal conditions to routinely measure peptides and proteins present in the extracellular space. However, the use of K⁺-induced depolarization can raise levels due to secretion and other processes to allow peptide sequencing and protein precursor identification in vivo. Significantly, subtraction of poor quality MS² spectra (i.e., basal conditions) from high quality MS² spectra (i.e., depolarization conditions) prior to database searching did not result in a loss of information. Rather, subtractive analysis simply reduced the number of MS² spectra requiring database searching from 3,349 to 859 and significantly reduced processing time ˜4-fold. This was confirmed by obtaining the same validated MS² spectra without subtractive analysis prior to database searching for several animals (n=3).

Spectral reproducibility for in vivo samples was determined by comparing data sets from different animals using subtractive analysis. This evaluation revealed that on average 32±15% or 132±22 MS² spectra collected per animal (specific to depolarization) were the same in comparing samples from any two animals. Spectral reproducibility in vivo was significantly lower than the 75% found for the tryptic digests discussed above. Interanimal variability can result not only from real differences between animals, but also experimental differences such as probe placement. The present inventors estimate the peptide concentrations to be 100-2000 pM (Haskins, W. E. et al. Analytical Chem., 2001, 73:5005-5014) corresponding to 360-7200 attomoles injected on-column in vivo. Because the estimated peptides concentrations were 1 nM corresponding to 400 attomoles injected on-column in vitro, it is improbable that the increased variability in vivo was simply due to lower levels of peptides in the dialysate than in the tryptic digest.

The results indicate that proteomics tools can be applied, with some degradation in performance, to dialysate samples despite the difficulties of limited samples and unknown protease specificity. A comparison of the results from the in vitro experiments to the in vivo experiments provides a sense of the limitations of the in vivo approach. Spectral reproducibility was 75±18% in vitro and 32±15% in vivo. This reduction in reproducibility was most likely due to inter-animal variability from real differences between animals and from experimental differences such as probe placement. Reproducibility of peptide sequencing and protein precursor identification was also markedly reduced in the in vivo experiments. For the in vitro experiments, 7 of 8 peptides were sequenced in 3 samples. In contrast, 0 to 9 peptides were sequenced in 13 animals. It is likely that greater reproducibility can be obtained in vivo by optimizing the gradient steepness parameter (Haskins, W. E. et al. Analytical Chem., 2001, 73:5005-5014), precursor ion windows and other data-dependent MS² scan functions (e.g. collision energy) for protein sequence coverage and by minimizing probe placement variability and, if possible, inter-animal variability.

In vivo samples were analyzed using both a 500-2000 m/z precursor ion window and a 550-600 m/z window. Using a 30-fold wider scan range, one would expect the presence of many more peptides to detect. With the narrower scan range, it was expected that greater sensitivity would be achieved allowing more low level peptides to be detected within this range. Since similar numbers of MS² spectra were collected and validated with both approaches, it appears that the two effects compensate each other. Thus, it is expected that maximal coverage of the dialysate proteome would be achieved by multiple injections of samples with narrow scan ranges (e.g., 50 m/z). However, this is a time-consuming process. The wider scan range allows peptides across the entire m/z range to be detected; albeit only the higher level peptides. Presumably the use of the wider scan function detects higher level peptides which may be of greater interest as possible functional peptides.

Single peptide sequences may be used for protein identification provided they are sufficiently validated. While single peptide sequences are often used for protein identification, they are seldom validated despite recent acknowledgement of false-positives using widely accepted scoring criteria (Peng, J. M. et al. J. Proteome Res., 2003, 2:43-50; MacCoss, M. J. et al. Analytical Chem., 2002, 74:5593-5599). Moreover, no rigorous examination of the false-positive rate for protein identification as a function of the number of peptides sequenced has been performed. The six step data-reduction strategy (FIG. 19) described in the experimental section yielded only 2% or 55 of 3,349 MS² spectra (29 were unique) that were validated by database searching. In step 4, MS² spectra remaining were considered validated by database searching if the same peptide sequence and protein precursor were identified by SEQUEST and MASCOT (step 4a), and if at least two peptides per protein precursor were found (step 4b). The overlap between the 1^{st} ranked SEQUEST-derived sequences (322 MS² spectra received a ΔCn score greater than 0.1 in step 3) and the 1^{st} ranked MASCOT-derived sequences (19% or 62 of 322 MS² spectra indicated homology with greater than 95% probability) was 17% or 55 of 322 MS² spectra (step 4a). All of these overlapping MS² spectra, 17% or 55 of 322, were found to correspond to at least two peptides per protein precursor (step 4b). Thus, Mascot effectively reduced false-positives, but no additional MS² spectra were eliminated by requiring at least two peptides per protein precursor. This suggests that the selection criteria in data-reduction step 4a were more stringent than the selection criteria in step 4b. In other words, it was unnecessary to require at least two peptides per protein precursor if the 1^{st} ranked SEQUEST- and MASCOT-derived sequences matched each other. Automated de novo sequencing provided further validation for 12% or 39 of 322 MS² spectra (step 5); however, in many cases it failed to validate MS² spectra despite validation by database searching programs (step 4). Thus, the selection criterium in step 5 was more stringent than the selection criteria in step 4. Library searching (step 6) revealed that only 3% or 10 of 322 significant MS² spectra (those that received a SEQUEST a ΔCn score greater than 0.1 in step 3) were incorrectly assigned by database searching. Again, the selection criterium in step 6 was more stringent than the selection criteria in previous steps. While it is possible that many of the 201 unique but not validated MS² spectra were properly assigned by SEQUEST, greater confidence in peptide sequencing and protein precursor identification at the attomole level was obtained by applying successively more stringent data-reduction steps. Therefore, comparing sequences obtained from two different database-searching programs (the 2^{nd} more stringent than the 1^{st}) and investigating additional validation by one de novo sequencing program was our preferred method of single peptide sequence validation and protein precursor identification. This is preferable to sequence validation by peptide synthesis or manual sequence interpretation, which are expensive and time-consuming, respectively.

TABLE 4
Summary of in vitro CLC-MS² results.

| | | | | Data-Reduction Step | | | | | | | | |
|---|---|---|---|---|---|---|---|---|---|---|---|---|
| | Spectral Reproducibility | | | 1 | 3 | | 4 | | 5 | | 6 | |
| | | | % | | | % | | % | | % | | % |
| Sample (n) | Samples | n − (n + 1) | Match | Total | No. | Total | No. | Total | No. | Total | No. | Total |
| 1 | 1-2 | 39 | 68 | 122 | 23 | 19 | 1-3, 5-8, YGGF_{D5}L (8) | 21 | 6 | 6 | 0 | 0 |
| 2 | 2-3 | 24 | 82 | 134 | 18 | 13 | 1-3, 5-8, YGGF_{D5}L (8) | 33 | 6 | 5 | 0 | 0 |
| 3 | 3-1 | 36 | 74 | 139 | 21 | 15 | 1-8, YGGF_{D5}L (9) | 25 | 7 | 6 | 0 | 0 |
| Total | N/A | 99 | N/A | 395 | 62 | N/A | 25 | N/A | 19 | N/A | 0 | N/A |
| Average | N/A | 33 | 75 | 132 | 21 | 16 | 8 | 26 | 6 | 6 | 0 | 0 |
| Standard Deviation | N/A | 8 | 7 | 9 | 3 | 3 | 1 | 7 | 1 | 0 | 0 | 0 |
| 95% Confidence | N/A | 20 | 18 | 22 | 6 | 7 | 1 | 16 | 1 | 1 | 0 | 0 |
| 1 | 2 | 3 | 4 | 5 | 6 | 7 | 8 | 9 | 10 | 11 | 12 | 13 |

Columns 1-13 are described from left to right. (1) The sample number. (2-4) Spectral reproducibility was calculated as described in the experimental section. The % match indicates the reproducibility between any two CLC-MS² datasets (n and n+1). (5-13) The number of MS² spectra remaining and the% of the total number of MS² spectra remaining in each data-reduction step (see experimental section). (8) The peptides (listed by the number assigned to the peptide in Table 5) and the total number of peptides observed in each sample (shown in parenthesis).

TABLE 5
Peptides and proteolytic processing patterns observed in vitro.

| | | | | | | | | | Protein Precursor |
|---|---|---|---|---|---|---|---|---|---|
| | | | | | | | | | (no.peptides, % aa |
| Peptide | Sample (n) | Database-Derived | De Novo-Derived Peptide | Position | Obs | Mr | Mr | | sequence coverage, |
| (n) | (no.peptides) | Peptide Sequence | Sequence | (aa) | (m/z) | (Expt) | (calc) | Delta | no.samples) |
| 1 | 1-3 (3) | FESNFNTQATNR | [276.1]LAKTNFAMTR | 34-45 | 715.3 | 1428.7 | 1427.6 | 1.0 | Lysozyme (3, 29%, 3) |
| 2 | 1-3 (3) | NTDGSTDYGILQINSR | [262.1]LLKLLGYDTVNSR | 46-61 | 878.0 | 1754.0 | 1752.8 | 1.2 | |
| 3 | 1-3 (3) | GTDVQAWIR | none | 117-125 | 523.8 | 1045.7 | 1044.5 | 1.1 | |
| 4 | 3 (1) | DFPIANGER | [262.1]PLANGER | 18-26 | 509.8 | 1017.5 | 1017.5 | 0.0 | Carbonic Anhydrase II |
| 5 | 1-3 (3) | ALVYGEATSR | none | 47-56 | 534.4 | 1066.7 | 1065.6 | 1.2 | (3, 11%, 3) |
| 6 | 1-3 (3) | VGDANPALQK | VGDAN[168.1]LKK | 148-157 | 506.8 | 1011.7 | 1011.5 | 0.1 | |
| 7 | 1-3 (3) | DGKGDVAFVK | DGAGGD[154.1]YVK | 200-209 | 518.4 | 1034.7 | 1034.5 | 0.2 | Conalbumin (2, 3%, 3) |
| 8 | 1-3 (3) | AQSDFGVDTK | [199.1]SDFRD[229.0] | 270-279 | 534.5 | 1067.0 | 1066.5 | 0.5 | |
| 1 | 2 | 3 | 4 | 5 | 6 | 7 | 8 | 9 | 10 |

Columns 1-10 are described from left to right. (1) The peptide number. (2) The sample (listed by the number assigned to the sample in Table 4) and the total number of samples that each peptide was observed in (shown in parenthesis). (3) Database-derived peptide sequences. (4) De novo-derived peptide sequences where regions of de novo-validated sequences with homology to database-derived sequences are underlined. (5) The amino acid positions of the peptide within the protein precursor. (6) The precursor ion's observed mass-to-charge ratio (m/z). (7) The experimental and (8) calculated monoisotopic mass of the peptide. (9) The difference between the experimental and calculated monoisotopic mass. (10) The protein precursors for 8 peptides in vitro. The number of peptides observed, the % sequence coverage by amino acid count and the total number of samples each peptide was observed in, are shown in parenthesis.

### Example 7

Investigation of Endogenous Neuropeptide Processing by in vivo Microdialysis-CLC-MS²

The objective of these experiments was to describe the biological significance of peptide sequences and protein precursors discovered in the ECF by in vivo microdialysis-capillary liquid chromatography (CLC)-tandem mass spectrometry (MS²). Previously, the present inventors demonstrated that CLC columns with integrated electrospray emitters operated at low nanoliter/min flow rates interfaced to quadrupole ion trap mass spectrometers could be used to monitor and discover endogenous peptides at attomole levels in microdialysis samples (Haskins, W. E. et al. Anal. Chem., 2001, 73:5005-5014). In Example 6, proteolytic processing (i.e., the production of peptides by the action of proteases on proteins) was investigated by in vivo microdialysis-CLC-data-dependent-MS². Here, proteolytic processing is investigated, as a function of the putative proteases involved in the cleavage of each peptide sequence from its protein precursor, of PEA and 5 other proteins whose peptide products were measured simultaneously. Evidence for biological activity of novel PEA-derived peptides discovered by this approach is also presented. These studies are expected to be particularly important for microdialysis-based proteomics applications in neuroscience.

A brief description of the results obtained in vivo is given here to facilitate a discussion of the biological significance of the peptide sequences and protein precursors discovered in this work. The development, evaluation and application of a proteomics-based data-reduction strategy in conjunction with the in vivo microdialysis-CLC-MS² system for discovering peptides are described in Example 6 and the Materials and Methods section.

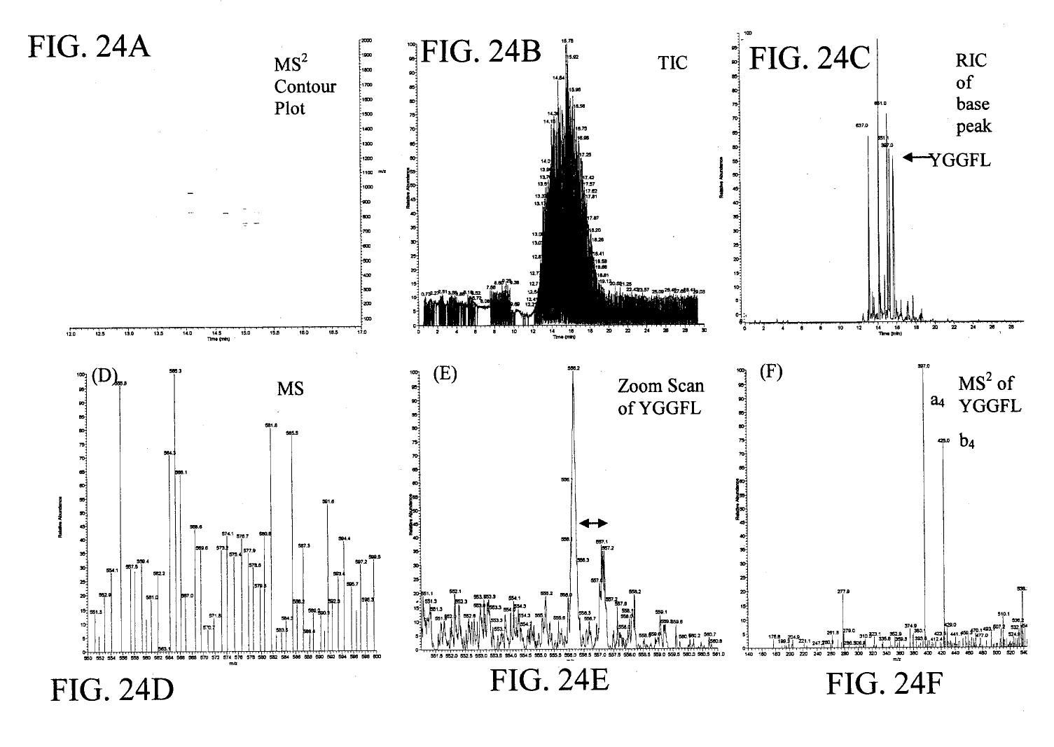

A total of 3,349 MS² spectra were collected from 13 different animals under basal conditions (with artificial CSF perfusing the probe) and during localized depolarization evoked by infusion of a high K⁺, low Na⁺ solution through the dialysis probe. Subtractive analysis revealed a total of 322 MS² spectra that were observed only during depolarization and received a significant score by database searching. From these MS² spectra, 29 peptide sequences and 6 protein precursors were identified using the database searching programs SEQUEST and MASCOT. FIGS. 24A-24F is an example of results obtained in vivo during depolarization-induced release of peptides into the ECF.

Stimulation by infusion of a high K⁺, low Na⁺ solution causes neuronal depolarization, Ca²⁺ influx, fusion of SVs with the plasma membrane, and release of the contents of SVs into the ECF by exocytosis (Yamamoto, Y. et al. Neuroscience Letters, 1997, 224:127-130). Therefore, the subtractive analysis method was expected to reveal only neuropeptides that were stored in neurons and released under de-polarizing conditions. Co-release of the known neuropeptides, the opioids Met- and Leu-enkephalin, along with 6 other PEA-derived peptides were found by this approach. Depolarization also caused increases in other peptides including neurogranin-, fibrinogen α and β and excitatory amino acid transporter 1-derived peptides.

The presence of these other peptides during K⁺-induced depolarization is of interest as not all of these were expected to be released from synaptic vesicles. One possibility is that depolarization raised the levels of proteases in the extracellular space which degraded the extracellular domains of synaptic or surface proteins and raised the levels of peptides in the ECF. Another possibility is that depolarization caused cell injury resulting in disruption of the blood brain barrier (BBB) and subsequent elevation of the levels of proteases, proteins and peptides in the ECF. Irrespective of the mechanism that raised the levels of peptides in the ECF during K⁺-induced depolarization, proteolytic processing can be described as a function of the putative proteases involved in the cleavage of each peptide sequence from its protein precursor.

The putative proteases involved in cleavage of 29 peptide sequences (27 novel) from 6 protein precursors are shown in Table 6. Proteases were identified for the N- and C-terminus cleavage sites of each peptide by searching the MEROPS protease database, which contains greater than 241 proteases for Rattus norvegicus and 539 for Homo sapiens (Rawlings, N. D. et al. Nucl. Acids, Res., 2002, 30:343-346). Serine-, cysteine-, thiol-, aspartic-, metallo-, amino- and carboxy-endopeptidases are all represented in Table 6. Many of the proteases in Table 6 have not yet been found in the rat brain; however, these are listed in Table 6 because genes encoding proteases are high conserved (Goumon, Y. et al. J. Biolog. Chem., 2000, 275:38355-38362) and many novel proteases with homology to these known proteases are expected to be discovered in forthcoming years. Proteases in bold are specific to 3 or more amino acids in the peptide sequence (starting one amino acid past the cleavage site), while other proteases are specific only to the 2 amino acids spanning the cleavage site. While it cannot definitively be determined which proteases were involved in cleavage of each peptide and protein by this approach, inspection of Table 6 by protease specificity, where bold proteases are the most significant, provides valuable information.

TABLE 6
Putative proteases involved in the proteolytic processing patterns observed in vivo.

| | | | Protein Precursor | Putative Peptidases from MEROPS Database | | | |
|---|---|---|---|---|---|---|---|
| Peptide | Position | | (no.peptides, % aa sequence | N-terminus | | C-terminus | C-Teminus |
| (n) | (aa) | Peptide Sequence | coverage, no.animals) | Cleavage Point | N-Teminus Peptidases | Cleavage Point | Peptidases |
| 1 | 100-104, | YGGFM (Met-enkephalin) | Preproenkephalin A (8, 28%, 8) | R/Y | Proprotein convertase 1, 2 | M/R | Cathepsin X |
| | 107-111, | | | K/Y | Polyporopepsin; Neurosporapepsin; | M/K | Carboxypeptidase |
| | 136-140, | Hyicolysin; Tryptase alpha; u- | | M; | | | |
| | 188-192, | Plasminogen activator; Angiotensin | | Pappalysin-1; | | | |
| | 212-216, | converting enzyme multipeptidase | | Kallikrein 1 | | | |
| | 263-267 | | | | | | |
| 2 | 118-122 | YPVEP | | R/Y | Proprotein convertase 1, 2 | P/E | Unknown |
| 3 | 118-125 | YPVEPEEE | | | | E/A | Gastricsin; |
| | | | | | | | Cathepsin E; |
| | | | | | | | Rhizopuspepsin; |
| | | | | | | | Phytepsin; |
| | | | | | | | Nepenthesin; |
| | | | | | | | Soytalidopepsin |
| | | | | | | | B; |
| | | | | | | | Aspergillopepsin |
| | | | | | | | II: Staphopain |
| | | | | | | | A; |
| | | | | | | | Aerugonolysin; |
| | | | | | | | Insulysin; |
| | | | | | | | Penicillolycin; |
| | | | | | | | Deuterolysin; |
| | | | | | | | Glutamyl |
| | | | | | | | aminopeptidase; |
| | | | | | | | Glycyl |
| | | | | | | | aminopeptidase; |
| | | | | | | | Proteinase |
| | | | | | | | K; Mername- |
| | | | | | | | AA054 |
| | | | | | | | peptidase; |
| | | | | | | | Archaean |
| | | | | | | | proteosome |
| | | | | | | | beta; Prohead |
| | | | | | | | proteinase |
| 4 | 198-207 | SPQLEDEAKE (Peptide I₁₋₁₀) | | R/S | Proprotein convertase 1, 2, 4, 5 and | E/L | Matrilysin |
| 5 | 198-209 | SPQLEDEAKELQ (Peptide I₁₋₁₂) | | | 7; Yapsin 1; Separase; Corin; | Q/K | Canditropsin; |
| | | | | | Kallikrein 1, mK1, mK6, mk9, rK1, rK10; | | Bontoxilysin; |
| | | | | | Snake venom factor V activator; | | CPG70 |
| | | | | | Complement factor | | carboxypeptidase |
| | | | | | 1, factorB, component2; Thrombin; | | |
| | | | | | Coagulation Factor C; Protein C; | | |
| | | | | | Falvivirin; PACE 4 proprotein | C | |
| | | | | | convertase; tripeptidyl-peptidase C | | |
| 6 | 219-229 | VGRPEWWMDYQ | | R/V | Cycstinyl aminopeptidase; dipeptidyl | Q/K | Canditropsin; |
| | | | | | peptidase III; Easter endopeptidase; | | Bontoxilysin; |
| | | | | | Coagluation factor VIIa, XIa, XIIa; | | CPG70 |
| | | | | | Plasma kallikrein; Acrosin; | | carboxypeptidase |
| | | | | | Hepatocyte growth factor activator; | | |
| | | | | | u- and t-Plasminogen activator; | | |
| | | | | | Intestinal Arg-specific | | |
| | | | | | endopeptidase | | |
| 7 | 232-236 | YGGFL (Leu-enkephalin) | | R/Y | Proprotein Convertase 1, 2 | L/K | Pepsin A; |
| | | | | | | | Renin; |
| | | | | | | | Cathespin D; |
| | | | | | | | Rhizopuspepsin; |
| | | | | | | | Barrier |
| | | | | | | | Pepsin; Felin |
| | | | | | | | immunodeficiency |
| | | | | | | | virus |
| | | | | | | | retropepsin; |
| | | | | | | | Carboxypeptidase |
| | | | | | | | M; CPG70 |
| | | | | | | | Carboxypeptidase |
| 8 | 252-260 | YSKEVPEME | | S/Y | Halylysin; Lysosomal dipeptidase I; | E/K | Olgopeptidase |
| | | | | | Acylaminoacyl-peptidase | | A; CPG70 |
| | | | | | | | Carboxypeptidase; |
| | | | | | | | Peptido- |
| | | | | | | | Lys- |
| | | | | | | | metalloendopeptidase |
| 9 | 53-78 | RKGPGPGGPGGAGGARGGAGGGPSGD | | G/R | Cathepsin B, X; Carboxypeptidase | None | N/A |
| | | | | | B,U; foot-and-mouth disease virus L | | |
| | | | | | proteinase: Aut2 peptidase; | | |
| | | | | | Leucolysin | | |
| 10 | 54-75 | KGPGPGGPGGAGGARGGAGGGP | Neurogranin (7, 33%, 7) | R/K | Proprotein convertase 2; Separase; | P/S | Ig A1-specific |
| 11 | 54-78 | KGPGPGGPGGAGGARGGAGGGPSGD | | | Carboxypeptidase U; Pitrilysin; | | serine |
| | | | | | Nardilysin; Hyicolysin; Tryptase | | endopeptidase |
| | | | | | alpha; Granzyme K; Complement | | |
| | | | | | factor D; Complement component | None | N/A |
| | | | | | activated C1s; Mannan-binding | | |
| | | | | | lectin-associated serine protease | | |
| | | | | | 1, 2; Protein C; Intestinal Arg-specific | | |
| | | | | | endopeptidase | | |
| 12 | 55-75 | GPGPGGPGGAGGARGGAGGGP | | K/G | Proprotein converatase 2; Foot-and- | P/S | Ig A1-specific |
| | | | | | mouth disease virus L proteinase | serine | |
| | | | | | endopeptidase | | |
| 13 | 55-76 | GPGPGGPGGAGGARGGAGGGPS | | | | S/G | Nuclear |
| | | | | | | | inclusion a- |
| | | | | | | | endopeptidase |
| 14 | 55-78 | GPGPGGPGGAGGARGGAGGGPSGD | | | | | |
| 15 | 57-78 | GPGGPGGAGGARGGAGGGPSGD | | P/G | Halylysin; Cytophagalysin; | None | N/A |
| | | | | | Membrane Pro-X carboxypeptidase; None | NA | |
| | | | | | Prolyl oligopeptidase; Dipeptidyl | | |
| | | | | | peptidase II | | |
| 16 | 20-36 | ADTGTTDEFIEAGGDIR | | A/A | Saccharolysin: Peptidyl dipeptidase | R/R | Proprotein |
| | | | | | Dcp; Gametolysin; Peptidase T; | | convertase 2, |
| | | | | | Bontoxilysin, Glycyl aminopeptidase; | | Omptin; |
| | | | | | Beta-Ala-His dipeptidase; | | Separase; |
| | | | | | Lysosomal dipeptidase I; Membrane | | Thimet |
| | | | | | Pro-X-carboxypeptidase; Pancreatic | | oligopeptidase; |
| | | | | | Elastase: Cytomegalovirus | | Carboxypeptidase |
| | | | | | assemblin; Signal peptidase 1 | | U; Insulysin; |
| | | | | | Dipeptidyl-peptidase II; C-terminal | | Nardilysin; |
| | | | | | processing protease-1 | | Penicillolysin; |
| 17 | 21-36 | DTGTTSEFIEAGGDIR | | A/D | Procollagen C-proteinase; | | Dipeptidyl- |
| | | | | Flavastacin | | peptidase III; | |
| 18 | 27-36 | EFIEAGGDIR | | S/E | Cathespin X | | BlaR1- |
| | | | | | | | peptidase; |
| | | | | | | | Kallikrein rK1, |
| | | | | | | | mK1, mK6, |
| | | | | | | mK9; Intestinal | |
| | | | | | | | Arg-specific |
| | | | | | | | endopeptidase |
| 19 | 228-236 | SPVPDLVPG | Fibrinogen Alpha Chain | M/S | Papain; Serralysn: Met-X | G/S | Cathepsin X; |
| | | | Precursos (5, 5%, 4) | | dipeptidase; Lysosomal dipeptidase | | Leishmanolysin; |
| | | | | | I | | Aeruginolysin; |
| | | | | | | | Halyllysin; |
| | | | | | | | Ecarin; |
| | | | | | | | Deuterolysin; |
| | | | | | | | Kallidin- |
| | | | | | | | releasing |
| | | | | | | | enzyme; |
| | | | | | | | Physarum |
| | | | | | | | serine carboxyl |
| | | | | | | | protease; |
| | | | | | | | Archaean |
| | | | | | | | proteasome- |
| | | | | | | | beta |
| | | | | | | | component; |
| | | | | | | | Meprin A |
| | | | | | | | peptidase |
| | | | | | | | complex |
| 20 | 240-250 | SQLQEGPPEWK | K/S | Trypsin I | K/A | Proprotein | |
| | | | | | | | convertase 1, 2; |
| | | | | | | | Penicillopepsin; |
| | | | | | | | Rhizopuspepsin; |
| | | | | | | | Rhodotorulapepsin; |
| | | | | | | | Bontoxilysin; |
| | | | | | | | Plasma |
| | | | | | | | Kallikrein |
| 21 | 13-32 | LVQTQAATTDSDKVDLSIAR | Fibranogen Beta Chain | S/L | Lysosomal dipeptidase I; Brevilysin | R/G | Proprotein |
| | | | Precursor (3, 4%, 1). | | L6; Horrilysin; Acutolysin; Fragilysin; | converatse 1, 2; | |
| | | | | | Rous sarcoma virus retropepsin | Thrombin; | |
| | | | | | | | Protein C; |
| | | | | | | | Clotting |
| | | | | | | | Enzyme; |
| | | | | | | | Flavirin; Furin; |
| | | | | | | | Peptidyl- |
| | | | | | | | glycinamdiase: |
| | | | | | | | Gaunidino |
| | | | | | | | benzoatase; |
| | | | | | | | Venombin AB; |
| | | | | | | | Angiotensin |
| | | | | | | | converting |
| | | | | | | | enzyme |
| | | | | | | | multipeptidase; |
| | | | | | | | Ancrod; |
| | | | | | | | Crotalase; |
| | | | | | | | Batroxobin; |
| | | | | | | | Trypsin 1: |
| | | | | | | | FoFibs |
| | | | | | | | endopeptidase; |
| | | | | | | | Deuterolysin; |
| | | | | | | | Hemerrhagic |
| | | | | | | | metalloproteinase |
| | | | | | | | a; Kistomin; |
| | | | | | | | Bilitoxin; |
| | | | | | | | Aeruginolysin; |
| | | | | | | | Gingipain R; |
| | | | | | | | Clostripain; |
| | | | | | | | Poliovirus-type |
| | | | | | | | picornain 2a; |
| | | | | | | | Grapevine- |
| | | | | | | | leaf-type |
| | | | | | | | nepovirus |
| | | | | | | | pecornain 3c |
| 22 | 20-31 | TTDSDKVDLSIA (Fibrinopeptide B₁₋₁₃) | | A/T | Presenelin 1; Tymovirus | A/R | Carboxypeptidase |
| | | | | | endopeptidase | | B, M, N, T, E, U; |
| | | | | | | | CPG70 |
| | | | | | | | carboxypeptidase; |
| | | | | | | | Dipeptidyl- |
| | | | | | | | peptidase 7; |
| | | | | | | | Carboxypeptidase |
| | | | | | | | D |
| | | | | | | | multipeptidase |
| 23 | 21-32 | TDSDKVDLSIAR (Fibrinopeptide B₂₋₁₄) | | T/T | Unknown | R/G | Proprotein |
| | | | | | | | converatse 1, 2; |
| | | | | | | | Thrombin; |
| | | | | | | | Protein C; |
| | | | | | | | Clotting |
| | | | | | | | Enzyme; |
| | | | | | | | Flavirin; Furin; |
| | | | | | | | Peptidyl- |
| | | | | | | | glycinamdiase; |
| | | | | | | | Gaunidino |
| | | | | | | | benzoatase; |
| | | | | | | | Venombin AB; |
| | | | | | | | Angiotensin |
| | | | | | | | converting |
| | | | | | | | enzyme |
| | | | | | | | multipeptidase; |
| | | | | | | | Ancrod; |
| | | | | | | | Crotalase; |
| | | | | | | | Batroxobin; |
| | | | | | | | Trypsin 1; |
| | | | | | | | PoFibs |
| | | | | | | | endopeptidase; |
| | | | | | | | Deuterolysin; |
| | | | | | | | Hemerrhagic |
| | | | | | | | metalloproteinase |
| | | | | | | | a; Kistomin; |
| | | | | | | | Bilitoxin; |
| | | | | | | | Aeruginolysin; |
| | | | | | | | Gingipain R; |
| | | | | | | | Clostripain; |
| | | | | | | | Poliovirus-type |
| | | | | | | | p cornain 2a; |
| | | | | | | | Grapevine- |
| | | | | | | | leaf-type |
| | | | | | | | nepovirus |
| | | | | | | | pecornain 3c |
| 24 | 526-543 | IAQDNEPEKPVADSETKM | Excitatory Amino Acid | L/I | Hyicolysin; Streptogrisin B | None | N/A |
| 25 | 528-543 | QDNEPEKPVADSETKM | Transporter 1 (4, 3%, 2) | A/Q | Procollagen I N-endopeptidase | | |
| 26 | 529-543 | DNEPEKPVADSETKM | | Q/D | Peptidyl-Asp metallo endopeptidase; | | |
| | | | | | Rice-tungro spherical virus-type | | |
| | | | | | endopeptidase | | |
| 27 | 531-543 | EPEKPVADSETKM | | N/E | Unknown | | |
| 28 | 191-211 | AKAPAPAAPAAEPQAEAPVAS | Brain Acidic Membrane Protein | A/A | Saccharolysin; Peptidyl dipeptidase | S/S | Vibriolysin; |
| | | | (2, 13%, 2) | | Dcp; Gametolysin; Peptidase T; | Lysosomal | |
| | | | | | Bontoxilysin, Glycyl | | dipeptidase I |
| 29 | 191-220 | AKAPAPAAPAAEPQAEAPVASSEQSVAVKE | | | aminopeptidase; Beta-Ala-His | None | N/A |
| | | | | | dipeptidase; Lysosomal dipeptidase | | |
| | | | | | I; Membrane Pro-X | | |
| | | | | | carboxypeptidase; Pancreatic | | |
| | | | | | Elastase; Cytomegalovirus | | |
| | | | | | assemblin; Signal peptidase I; | | |
| | | | | | Dipeptidyl-peptidase II; C-terminal | | |
| | | | | | processing protease-1 | | |
| 1 | 2 | 3 | 4 | 5 | 6 | 7 | 8 |

Proteolytic processing patterns are described as a function of the putative proteases involved in N- and C-terminus bond cleavage of each peptide sequence from its protein precursor. Proteases in bold are specific to 3 or more amino acids in the peptide sequence (starting one amino acid past the cleavage site).

Based on observations of specific peptides during K⁺-induced depolarization and the observations of others, the serine proteases appear to be particularly important in the ECF of the brain. Serine proteases, their natural inhibitors, serpins, and their receptors are found throughout the brain (Vernigora, A. N. and M. T. Gengin Biochemistry-Moscow, 1996, 61:555-564; Turgeon, V. L. and Houenou, L. J. Brain Res., 1997, 25:85-95). These enzymes contain a catalytic site consisting of a serine, histidine and aspartic acid residue. The exact proteases involved in the production of the 29 peptides remain to be conclusively determined. However, co-release of PEA-derived peptides (e.g., Met- and Leu-enkephalin) and previously unknown fibrinopeptides (e.g., fibrinopeptide B₁₋₁₃ and B₂₋₁₄) suggest that the serine proteases (e.g., PC1/3, PC2 and thrombin) are exceptionally important in the ECF of the brain.

The PEA-derived opioids, including Met- and Leu-enkephalin, are produced by the action of PC1/3, PC2 and numerous other proteases. PC 1/3 and PC2 are calcium-dependent, serine proteases that are known to cleave PEA at mono- and di-basic sites in the Ca²⁺ rich, acidic environment of SV in the brain (Rouille, Y. et al. Frontiers in Neuroendocrinology, 1995, 16:322-361). Differential processing of PEA by PC1/3 and PC2 (Breslin, M. B. et al. J. Biolog. Chem., 1993, 268:27084-27093) corresponding with different distributions in neuronal tissue (e.g., striatum) and cell lines (Lindberg, I. et al. Mol. Cell. Neuroscience, 1994, 5:614-622) has also been shown. Eight PEA-derived peptides, 6 novel peptides and Met- and Leu-enkephalin, were observed in the ECF of the brain by in vivo microdialysis-CLC-MS².

Seven of the 8 PEA-derived peptides observed support the general mechanism for proteolytic processing. FIG. 12 shows the amino acid sequence of PEA and PEA-derived peptides discovered in this work. None of the 8 peptides observed originating from PEA (269 aa) were tryptic peptides (i.e., C-terminal R or K). This was expected as carboxypeptidase (e.g., carboxypeptidase C or H) cleavage of C-terminal mono- and di-basic amino acid residues following endopeptidase (e.g., PC1/3 and PC2) cleavage at mono- and di-basic sites is well-documented for PEA. Only 3 of the 8 peptides observed (i.e., peptide 1-YGGFM or Met-enkephalin, peptide 7-YGGFL or Leu-enkephalin and peptide 6-VGRPEWWMDYQ) can be explained solely by PC1/3 and PC2 cleavage. Four of the remaining 5 peptides (peptides 2-5 in Table 6) can be explained by PC1/3 and PC2 cleavage followed by carboxypeptidase cleavage.

However, peptide 8 cannot be explained simply by the action of the serine protease, acylaminoacyl peptidase, followed by carboxypeptidase cleavage (Table 6). Acylaminoacyl peptidase cleaves and acetylates the N-terminus of α-melaocyte stimulating hormone (α-MSH) in the brain, but peptide 8 was found to be unmodified. Therefore, another mechanism or unknown protease must produce peptide 8. Table 6 indicates that the metallopeptidase, lysosomal dipeptidase I, with specificity for cleaving SY bonds, is a good possibility.

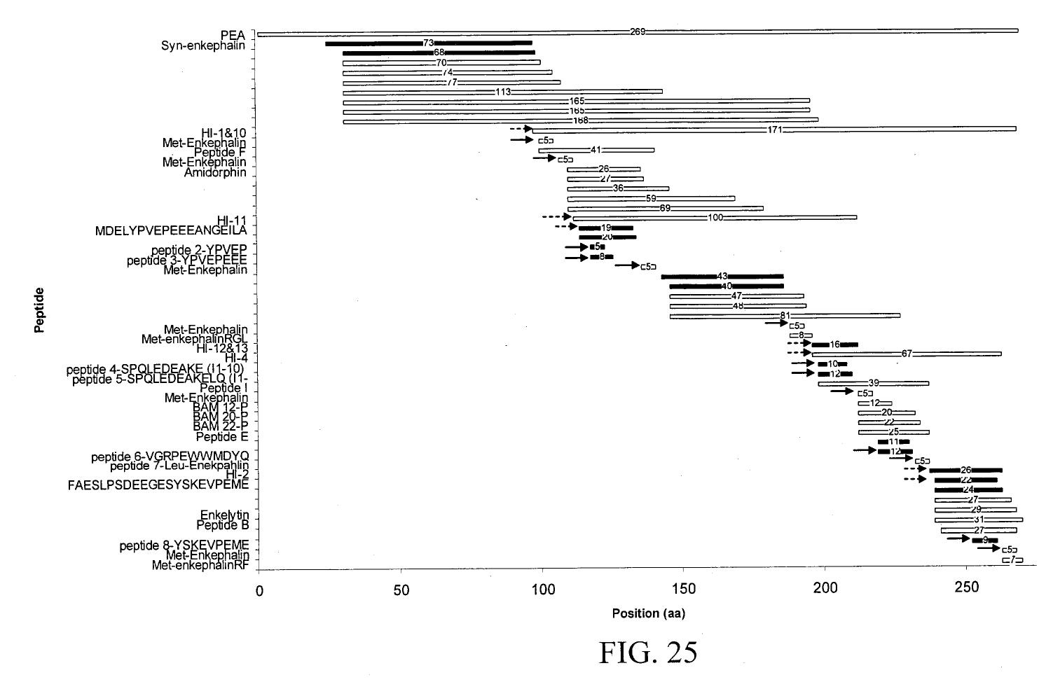

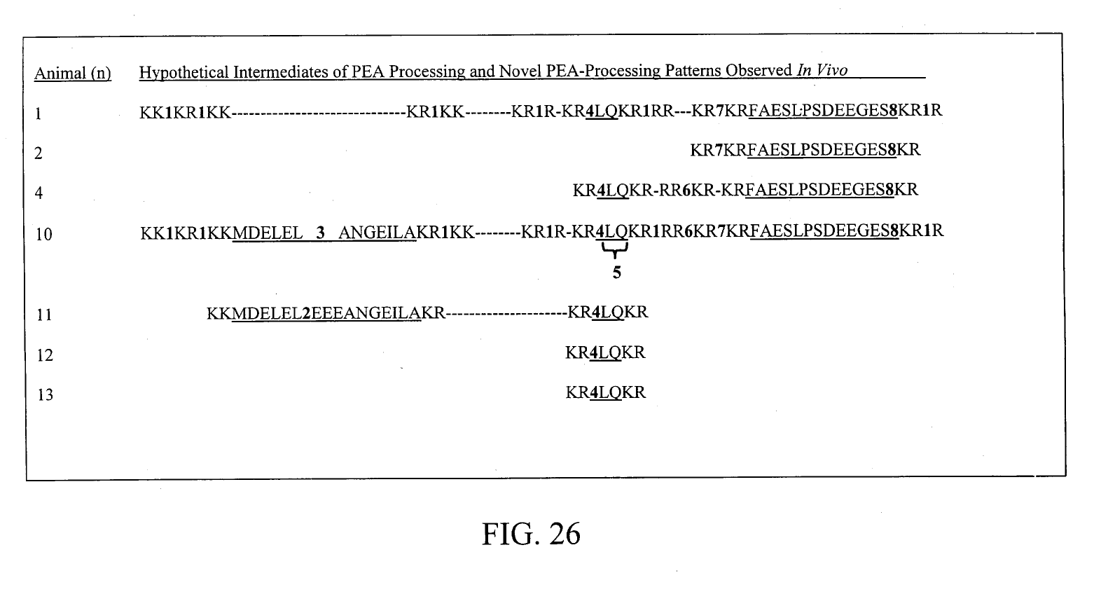

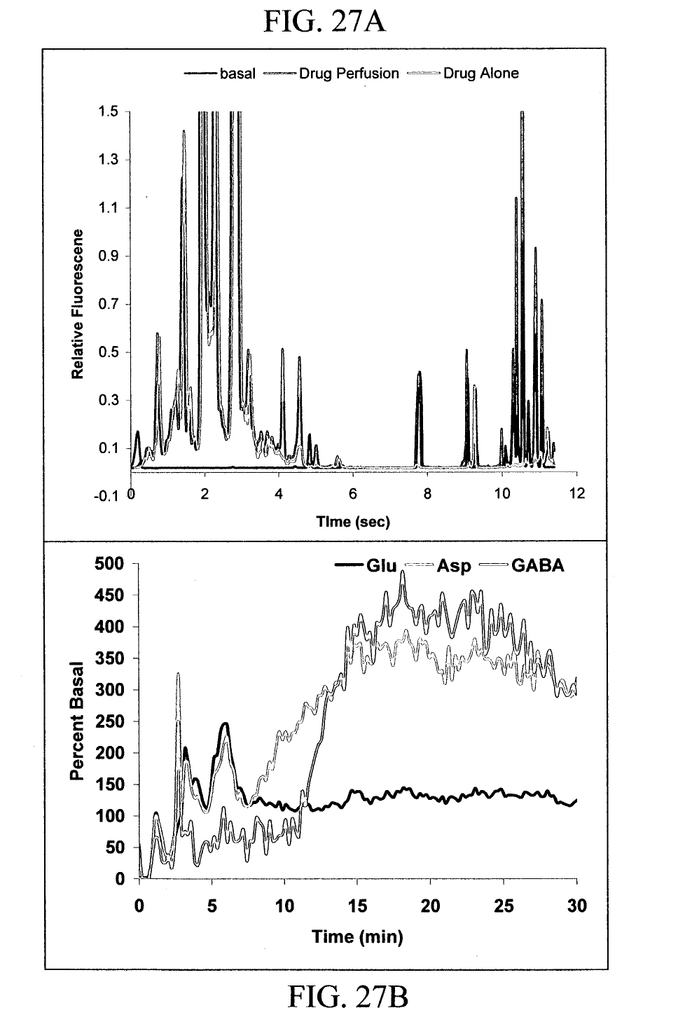

Extensive prior studies in brain tissue (Liston, D. et al. Science, 1984, 225:734-737; Fayada, C. et al. Neuropeptides, 1987, 9:9-17; Patey, G. et al. Life Sciences, 1983, 33:117-120; Patey, G. et al. Febs Letters, 1984, 172:303-308; Patey, G. et al. Neuroscience, 1985, 15:1035-1044) and bovine chromaffin cells (Wang, H. P. and Dass,C. Peptides, 2002, 23:2143-2150; Hook, V. and Metz-Boutigue,M. H. Chromaffin Cell: Trnsmitter Biosynthesis, Storage, Release, Actions, and Informatics, 2002, 971:397-405; Lembo, P. M. C. et al. Nature Neuroscience, 2002, 5:201-209; Goumon, Y. et al. Journal of Biological Chemistry, 2000, 275:38355-38362; Condamine, E. et al. Peptides, 1999, 20:865-871; Hook, V. Y. H. et al. Endocrinology, 1999, 140:3744-3754; Yasothornsrikul, S. et al. Biochemistry, 1999, 38:7421-7430; Goumon, Y. et al. Journal of Biological Chemistry, 1998, 273:29847-29856; Krieger, T. J. et al. Journal of Neurochemistry, 1992, 59:26-31; Wilson, S. P. et al. Journal of Neurochemistry, 1991, 57:870-875; Mindroiu, T. et al. Biochimica et Biophysica Acta, 1991, 1076:9-14; Ase, A. et al. Medicina-Buenos Aires, 1987, 47:606-607; Birch, N. P. and Christie, D. L. Journal of Biological Chemistry, 1986, 261:2213-2221; Watkinson, A. and Dockray, G. J. Regulatory Peptides, 1987, 18:350; Dumont, M. and Lemaire, S. Federation Proceedings, 1987, 46:1448; Birch, N. P. and Christie, D. L. Journal of Biological Chemistry, 1986, 261:2213-2221) have not revealed the presence of these 6 novel PEA-derived peptides. Because most previous work on proteolytic processing used antibodies to select peptides with certain (Cupo, A. et al. Neuropeptides, 1984, 4:389-401) epitopes, and because these peptides do not contain any homology with Met- or Leu-enkephalin, it is unlikely that these peptides would have been detected by any conventional processing studies. FIG. 25 shows a non-exhaustive depiction of selected PEA-derived peptides by amino acid position. Unshaded peptides contain the sequence YGGFX where X is M or L while shaded peptides do not. Peptides observed in this work are indicated by an arrow and hypothetical intermediates (HIs) deduced from mono- and di-basic cleavage sites are indicated by a dashed arrow. Clearly, in vivo microdialysis-CLC-MS² has revealed a previously unknown subset of PEA-derived peptides without homology to Met- or Leu-enkephalin.

HIs for all of the PEA-derived peptides observed in this work are shown in FIGS. 25 and 26 for the 7 of 13 animals where PEA processing was observed (i.e., animals 1,2,4 and 10-13 in Table 2). An HI is defined herein as the smallest precursor peptide (i.e., propeptide) containing all of the peptide sequences observed in each animal. Thus, HIs are relatively large peptides containing all of the peptide sequences observed in each animal. HIs are shown with and without (underlined) mono- or di-basic sites in FIGS. 25 and 26 for completeness. Predicted mono- and di-basic cleavage sites (i.e., K, R, KR, KK and RR), the peptides observed (bold and numbered by amino acid position according to Table 6) and selected other amino acids comprising the HI sequences (e.g., Q for peptides 4-7) are also shown (There are 6 copies of peptide 1-YGGFM (Met-enkephalin) in PEA and only 1 copy of peptide 7-YGGFL (Leu-enkephalin) as shown in FIG. 12. Which copy of Met-enkephalin that was observed in the ECF cannot be determined by this approach; therefore, all amino acid positions are shown in FIGS. 25 and 26). Five HIs with mono- or di-basic sites and 3 without (underlined) were deduced from the peptide sequences observed in each animal (FIG. 26). Spectral reproducibility studies in vitro and in vivo suggest that variability in the peptide sequences observed and the HIs predicted are, in fact, due to interanimal variability.

The HI for peptide 4-SPQLEDEAKE (peptide I₁₋₁₀), peptide 5-SPQLEDEAKELQ (peptide I₁₋₁₂), is produced in the ECF of the brain (FIG. 26). Peptide 4-SPQLEDEAKE (peptide I₁₋₁₀) is an N-terminal cleavage product of peptide I, and it does not appear to have been produced from peptide I by bond cleavage of both the N- and C-terminus at mono- and di-basic sites that is typically associated with endopeptidase activity (e.g., YGGFM and YGGFL). The N-terminus appears to have been produced by cleavage at a dibasic site (i.e., KR); the C-terminus does not (i.e., LQ). Previously, the present inventors hypothesized that the production of peptide I₁₋₁₀ occurs by endopeptidase (e.g., PC 1/3 and 2) cleavage of peptide I-SPQLEDEAKELQKRYGGFMRRVGRPEWWMDY, a known product of PEA-processing in purified BAM cells (Hunt, D. F. et al. Proc. Natl. Acad. Sci. USA, 1986, 83:6233-6237), to yield the intermediate SPQLEDEAKELQ (SEQ ID NO: 5) which is then cleaved by a carboxypeptidase to yield the SPQLEDEAKE (SEQ ID NO: 4) sequence detected in vivo. This pathway seemed likely, given the di-basic site in peptide I and the apparent ubiquity of exopeptidase activity in the brain extracellular space (Viklund, C. et al. Chem. of Materials, 1997, 9:463-471). Measurement of peptide 5-SPQLEDEAKELQ (SEQ ID NO: 5) (peptide I₁₋₁₂) in the ECF provides evidence for the proteolytic processing mechanism proposed above.

Other HIs may also be observed by in vivo microdialysis-CLC-MS². Relatively small HIs without mono- or di-basic sites (e.g., MDELYPVEPEEEANGEILA for peptides 2 and 3 and FAESLPSDEEGESYSKEVPEME for peptide 8) are amenable to sequencing by MS². However, many of the HIs with mono- or di-basic sites shown in FIGS. 25 and 26 (e.g., HI-11 or KKMDELEL2EEEANGEILAKR - - - KR4LQKR for peptide 2) are too large (e.g., 100aa for HI-ll) for peptide sequencing by low energy collision-induced dissociation (CID) in the QIT. Thus, these experiments revealed a previously unknown subset of all possible bioactive peptides and intermediates in the ECF.

Comparison of these results for PEA-processing in brain ECF with recent results for PEA-processing in BAM cells (Goumon, Y. et al. J. Biol. Chem., 2000, 275:38355-38362) and brain tissue (Skold, K. et al. Proteomics, 2002, 2:447-454) illustrates the importance of live animal studies for discrimination between intracellular peptides and those that are actually released into brain ECF. In the BAM cell study, preparative scale HPLC, antibodies for specific PEA-derived peptides, Edman sequencing and matrix-assisted laser desorption/ionization-time of flight mass spectrometry (MALDI-TOFMS) were combined to reveal 30 PEA-derived peptides (Goumon, Y. et al. J. Biol. Chem., 2000, 275:38355-38362). In addition, several phosphorylated peptides were also found. All of the unmodified peptides that were discovered in that work are contained within FIG. 4 (i.e., renumbered for the 30 amino acid signal peptide in PEA). The putative proteases cited for the production of these peptides included PC1/3, PC2, PAM, prohormone thiol-protease, propiomelanocortin-converting enzyme, serine proteases and carboxypeptidases. Five of the 30 peptides, corresponding to 3 novel cleavage sites (i.e., LA at aa positions 99-100, AK at 100-1001 and ME at 259-260) observed in the ECF during nicotine stimulation, were from the N- and C-terminal domain of PEA. In the brain tissue study, focused microwave irradiation and CLC-MS² were combined to reveal 550 endogenous peptides in the hypothalamus (Skold, K. et al. Proteomics, 2002, 2:447-454). Also, several peptides with various PTMs were found. Nine unmodified PEA-derived peptides, corresponding to 3 novel cleavage sites (i.e., RS at 197-198, RV at 218-219 and YG at 263-264), spanned almost the entire domain of PEA. The putative proteases cited for production of these peptides included convertases, such as PC2, and carboxypeptidases. Interestingly, a PC2-derived peptide was also observed in that work.

None of the 30 PEA-derived peptides from the BAM cell study and only 4 of the 9 PEA-derived peptides from the brain tissue study (peptides 1, 4, 6 and 7) were observed in the present experiments. Moreover, the 8 peptides observed in the present experiments in brain ECF during K⁺-stimulation, corresponding to 6 novel cleavage sites (i.e., LY at aa positions 117-118 for peptides 2 and 3, RS at 197-198 for peptides 4 and 5, RV at 218-219 for peptide 6 and SY at 251-252 for peptide 8), spanned almost the entire domain of PEA.

Many of the BAM studies used trypsin followed by carboxypeptidase B cleavage to generate PEA-derived peptides for bioactivity assays such as the guinea pig ileum or opiate receptor binding assays. These and other PEA-derived peptides were found to be important in a large number of physiological and behavioral functions including pain and analgesia, immunity, appetite regulation and emotion, tolerance and dependence, mental illness and cardiovascular response (Bodnar, R. J. and Hadjimarkou, M. M. Peptides, 2002, 23:2307-2365).

The presence of peptides derived from PEA in the ECF is not surprising given the expression of this protein in the brain and the expectation that peptides “left-over” from the production of Met- and Leu-enkephalin by PEA-processing would be present in neuronal vesicles and co-released with peptides of known activity. It has been suggested that PEA-derived peptides without bioactivity may direct bioactive peptides to specific target sites (Jones, B. N. et al. Proc. Natl. Acad. Sci. USA, 1982, 79:1313-1315).

In order to determine bioactivity of PEA-derived peptides, peptide 11lo was infused into the dialysis probe while simultaneously monitoring the levels of neuroactive amino acids such as γ-aminobutryric acid (GABA), glutamate, aspartate, glycine, and serine by in vivo microdialysis-capillary electrophoresis with laser-induced fluorescence detection (n=2) (Bowser, M. T. and Kennedy, R. T. Electrophoresis, 2001, 22:3668-3676). An increase in aspartate and GABA was observed upon infusion of 10 μM peptide I₁₋₁₀ while glutamate levels remained the same.

GABA and its precursor, glutamate, are the major inhibitory and excitatory neurotransmitters in the brain, respectively. Consequently, the balance between these two is a subject of intense pharmacological investigation (Watanabe, M. et al. International Review of Cytology—A Survey of Cell Biology, 2002, 213:1-47; Stein, V. and Nicoll, R. A. Neurochemical Research, 1998, 23:563-570; Petroff, O. A. C. Neuroscientist, 2002, 8:562-573; Owens, D. F. and Kriegstein, A. R Nature Reviews Neuroscience, 2002, 3:715-727; Osborne, P. G. et al. Journal of Neuroscience Methods, 1990, 34:99-105). Previous microdialysis studies have shown that Leu-enkephalin activates opioid receptors and inhibits nicotine-stimulated GABA production (Nong, Y. et al. Brain Res., 2003, 961:45-52). In contrast, apomorphine stimulates GABA production while glutamate levels remain the same (Grobin, A. C. and Deutch, A. Y. J. Pharma. Exper. Therapeutics, 1998, 285:350-357). Receptor assays probing the binding of peptide I₁₋₁₀ to opioid receptors proved negative (data not shown). Therefore, activity of peptide I₁₋₁₀ is not mediated by the opioid receptors. Based on these results, it is hypothesized that peptide I₁₋₁₀-stimulated GABA release is involved in the regulation of other bioactive peptides such as Met- and Leu-enkephalin.

Observed were peptides from the C-terminal domain of neurogranin (NG) (Slemmon, J. R. et al. Molecular Neurobiology, 2000, 22:99-113; Mons, N. et al. Journal of Neurochemistry, 2001, 79:859-867; Li, G. L. et al. Apmis, 2000, 108:98-106; Gerendasy, D. D. and Sutcliffe, J. G. Molecular Neurobiology, 1997, 15:131-163; Chakravarthy, B. et al. Trends in Neurosciences, 1999, 22:12-16; Baudier, J. et al. Journal of Biological Chemistry, 1991, 266:229-237), brain acidic membrane protein (BAMP) (Yamamoto, Y. et al. Neuroscience Letters, 1997, 224:127-130; Maekawa, S. et al. Journal of Biological Chemistry, 1993, 268:13703-13709; lino, S. et al. Neuroscience, 1999, 91:1435-1444) and excitatory amino acid transporter 1 (EAAT1) (Gegelashvili, G. and Schousboe, A. Molecular Pharmacology, 1997, 52:6-15) in the ECF of the brain. All of these proteins are substrates for protein kinase C (PKC) involved in synaptic plasticity and neurotransmitter release (Slemmon, J. R. et al. Molecular Neurobiology, 2000, 22:99-113;Gegelashvili, G. and Schousboe, A. Molecular Pharmacology, 1997, 52:6-15). However, none of these proteins have any known peptide products. Seven novel peptides derived from NG, 2 from BAMP, and 4 from EAAT1 were discovered by in vivo microdialysis-CLC-MS².

It is hypothesized that the peptides observed from the C-terminal domains of these proteins are from extracellular domains that were exposed to proteases during depolarization. However, the orientation of NG remains unknown and the discovery that the C-terminal domain of BAMP resides on the external surface of SVs (Yamamoto, Y. et al. Neuroscience Letters, 1997, 224:127-130) does not support this theory. Moreover, the existing models for EAAT1 (Gegelashvili, G. and Schousboe, A. et al. Molecular Pharmacology, 1997, 52:6-15; Mitrovic, A. D. et al. Journal of Biological Chemistry, 1998, 273:14698-14706), suggest that the C-terminal domain is intracellular, rather than extracellular, during depolarization-induced transporter-reversal (Raiteri, L. et al. Journal of Neurochemistry, 2002, 80:706-714 (2002); Phillis, J. W. et al. Brain Research, 2000, 868:105-112). Thus, no support for this hypothesis was provided by previous work on these proteins, and further investigations will be required.

NG and BAMP belong to a small family of acidic, synaptic proteins that bind and sequester calmodulin (CaM) (Yamamoto, Y. et al. Neuroscience Letters, 1997, 224:127-130; Chakravarthy, B. et al. Trends in Neurosciences, 1999, 22:12-16). Neuronal depolarization leads to Ca²⁺ influx, phosphorylation of these proteins by PKC, release of CaM, and Ca²⁺-CaM stimulated release of SVs (Chakravarthy, B. et al. Trends in Neurosciences, 1999, 22:12-16). Therefore, it is hypothesized that NG and BAMP are Ca²⁺ bound, unmodified and free of CaM during depolarization (Chakravarthy, B. et al. Trends in Neurosciences, 1999, 22:12-16). There were no NG- or BAMP-derived peptides, phosphorylated or unmodified, containing known phosphorylation sites for PKC observed. However, the knowledge that the excitatory neurotransmitter glutamate is released from SVs during depolarization, combined with observations such as glutamate-stimulated NG phosphorylation (Rodriguez Sanchez, P. et al. Neuroscience Letters, 1997, 221:137-140) and enhanced NG phosphorylation during long-term potentiation (Chen, S. J. et al. Brain Research, 1997, 749:181-187), provide evidence in support of this hypothesis.

NG-, BAMP-, and EAAT1-derived peptides support the general mechanism for proteolytic processing. Five of the 7 NG-derived peptides (peptides 10-14 in Table 6) that were observed in the ECF of the brain can be explained either solely by PC2 cleavage or by PC2 cleavage followed by carboxypeptidase cleavage. The remaining 2 peptides (peptides 9 and 15 in Table 6) can be explained by various endopeptidases (e.g., the cysteine protease, cathepsin B) and by the serine protease dipeptidyldipeptidase II, respectively. The 2 BAMP-derived peptides (peptides 28 and 29 in Table 6) can be explained by various endopeptidases (e.g., the metallopeptidase, bontoxilysin) and, in the case of peptide 28, this is followed by carboxypeptidase cleavage. Three of the 4 EAAT1-derived peptides (peptides 24-26 in Table 6) can be explained by various endopeptidases (e.g., the serine protease, streptogrisin B). However, no putative proteases for peptide 27-EPEKPVADSETKM, cleaving the NE bond at the N-terminus, were found in the MEROPS database. This suggests that a novel protease, in addition to the novel cleavage site at aa positions 530-531, may have been discovered.

Decreased levels of NG (78 aa) have been observed during sleep-deprivation (Neunerjehle, M. et al. Brain Research, 1995, 685:143-153), aging (Mons, N. et al. Journal of Neurochemistry, 2001, 79:859-867), and brain injury studies (Li, G. L. et al. Apmis, 2000, 108:98-106) while very little is known about BAMP (220 aa). Various calcium-dependent cysteine proteases (e.g., calpain and caspase) released into the ECF during cell death may explain the decreased levels of NG observd in these earlier studies (Wang, K. K. W Trends in Neurosciences, 2000, 23:20-26). However, NG and BAMP are not known substrates for these proteases, and no evidence for the involvement of these proteases was found in this work.

EAAT1 (543 aa) is a Na⁺-dependent glutamate/aspartate transport protein that mediates glutamate uptake mechanisms using a Na⁺, K⁺ electrochemical gradient as a driving force (Gegelashvili, G. and Schousboe, A. Molecular Pharmacology, 1997, 52:6-15). Glutamate uptake is enhanced during EAAT1 phosphorylation by PKC. It is hypothesized that EAAT1 ceased to transport excess glutamate, possibly causing neurotoxic degeneration, disruption of the BBB and cell death. This is supported by greatly reduced extracellular Na⁺ levels during K⁺-induced depolarization.

Fibrinogen (550 aa) is involved with thrombin in blood coagulation, and fibrinogen processing has been studied considerably in blood (Blomback, B. et al. Acta Chemica Scandinavica, 1965, 19:1789-; Blomback, B. et al. Acta Chemica Scandinavica, 1965, 19:1788-; Tegemil, A. C. and Blomback, B. Acta Chemica Scandinavica, 1967, 21:307-). It is composed of 3 polypeptide chains (α, β and γ) and is proteolytically processed to fibrinopeptide A (α-derived) and fibrinopeptide B (β-derived) following binding to the protease thrombin. Fibrinopepides A and B assemble fibrin which coagulates blood by forming a polymeric clot. Fibrinogen-derived peptides are particularly important in diabetes (Ceriello, A. Diabetologia, 1993, 36:1119-1125) and cancer (Rickles, F. R. et al. Cancer and Metastasis Reviews, 1992, 11:237-248) research. Five novel peptides derived from fibrinogen α and 3 from fibrinogen β were discovered in this work.

Fibrinogen-derived peptides support the general mechanism for proteolytic processing. All of the 5 fibrinogen 0:-derived peptides (peptides 16-20 in Table 6) can be explained by various endopeptidases (e.g., the serine protease kallikrein). Two of the 3 fibrinogen β-derived peptides (peptides 21-22 in Table 6) can be explained by various endopeptidases (e.g., the serine protease thrombin that produces fibrinopeptide B). However, no putative proteases for peptide 23-TDSDKVDLSIAR, cleaving the TT bond at the N-terminus, were found in the MEROPS database. This suggests that a novel protease, in addition to the novel cleavage site at aa positions 530-531, may have been discovered.

Two of the 3 fibrinogen β-derived peptides, peptide 22-TTDSDKVDLSIA (fibrinopeptide B₁₋₁₃) and peptide 23- TDSDKVDLSIAR (fibrinopeptide B₂₋₁₄), are homologous with fibrinopeptide B (TTDSDKVDLSTAR), suggesting that depolarization caused disruption of the BBB and these peptides are involved in blood coagulation. While this remains a distinct possibility, the serine proteases have recently been shown to participate in various neuronal activities (Turgeon, V. L. and Houenou, L. J. Brain Research Reviews, 1997, 25:85-95).

For example, proteolytically activated receptors (PARs), were found to be cleaved by thrombin (Turgeon, V. L. and Houenou, L. J. Brain Research Reviews, 1997, 25:85-95; Mackie, E. J. et al. Iubmb Life, 2002, 53:277-281; Wang, H. and Reiser, G. Biological Chemistry, 2003, 384:193-202). PARs are activated by cleavage of an extracellular domain to form a new N-terminus which acts as a tethered ligand to bind to another extracellular domain for intracellular signaling. This phenomenon supports the hypothesis that the peptides observed from the C-terminal domains of NG, BAMP and EAAT1 are from extracellular domains that were exposed to proteases during depolarization. It also suggests the possibility that these (e.g., fibrinogen) and other proteins may also be proteolytically activated.

The biological significance of the 29 peptide sequences and 6 protein precursors discovered by in vivo microdialysis-CLC-MS² in the ECF during K⁺-induced depolarization remains unknown because the exact proteases involved and the mechanism that raised the levels of peptides in the ECF during K⁺-induced depolarization remain unknown. However, examination of the peptide sequences as a function of the putative proteases involved provides important information. Depolarization caused increases in 8 PEA-derived peptides (corresponding to 6 novel peptides and 6 novel cleavage sites) and other peptides including neurogranin-, fibrinogen α and β- and excitatory amino acid transporter 1-derived peptides. Co-release of PEA-derived peptides (e.g., Met- and Leu-enkephalin) and previously unknown fibrinopeptides (e.g., fibrinopeptide B₁₋₁₃ and B₂₋₁₄) suggest that the serine proteases (e.g., PC1/3, PC2 and thrombin) are exceptionally important in the ECF of the brain. Seven of the 8 PEA-derived peptides can be explained via cleavage by PC1/3 and PC2 followed by carboxypeptidase cleavage (e.g., carboxypeptidase H or C), supporting the general mechanism for proteolytic processing. Bioactivity of peptide I₁₋₁₀, a novel PEA-derived peptide, and co-release of known neuropeptides (i.e., the opioids Met- and Leu-enkephalin) suggests that important inter-neuron signaling mechanisms have been discovered.

### Example 8

### Identification of Localized Neurochemical Changes

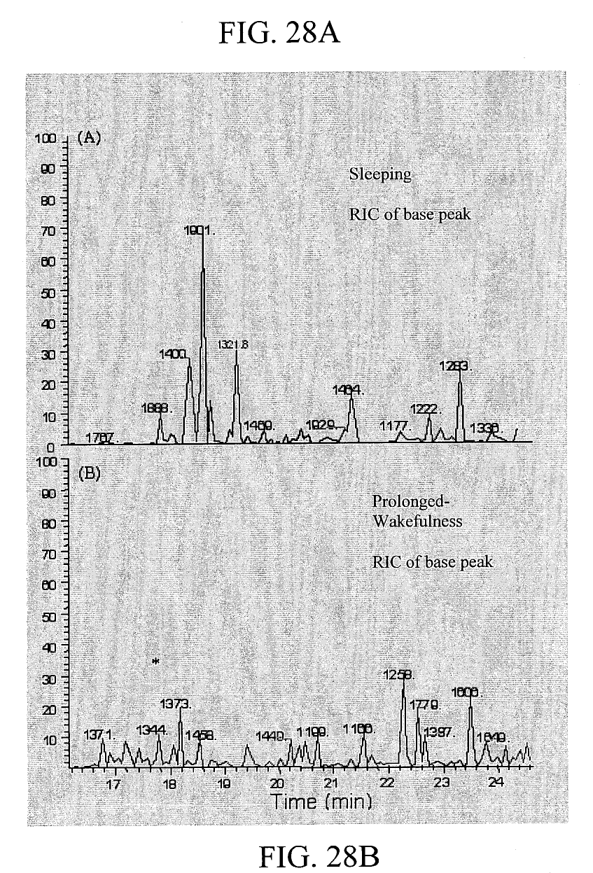

The apparatus and methods of the present invention can be used to identify localized neurochemical changes that occur in response to any physiological or behavioral state. This application was investigated by comparing in vivo microdialysis-CLC-MS² data obtained from animals during sleep and prolonged wakefulness as shown in FIGS. 28A-28B (Strecker, R. E. et al. Neuroscience, 1987, 22:169-178). While the detected compounds have yet to be identified, the data clearly illustrate a differential peptide profile in the extracellular space under these two conditions. Detection of these chemical changes could be used to identify compounds that control the sleep state. One can envision many applications for identifying biomarkers and/or signaling molecules of behaviors or physiological states using the apparatus and methods of the present invention.

Preparative 2-dimensional electrophoresis of human cerebral spinal fluid followed by trypsin proteolysis and matrix-assisted laser desorption/ionization-time of flight mass spectrometry (MALDI-TOFMS) revealed 21 proteins Onot previously observed by traditional methods (Davidsson, P. et al. Proteomics, 2001, 1:444-452) and CLC-MS² has allowed identification of over 1500 peptides in brain tissue samples (Skold, K. et al. Proteomics, 2002, 2:447-454). These techniques offer complementary sequence information for in vivo microdialysis-CLC-MS² studies (from peptides that ionize well by MALDI and from peptides found in brain tissue, respectively). Another interesting approach is the detection of neuropeptides in carboxypeptidase E deficient (i.e., ‘knockout’) mice. The lack of this protease results in elevated levels of neuropeptides which facilitated identification of many novel peptides in tissue samples by CLC-MS² (Che, F. Y. et al. Proc. Natl. Acad. Sci. USA, 2001, 98:9971-9976). The combined use of CE with MALDI-MS to analyze dialysates (Zhang, H. et al. Journal of Mass Spectrometry, 1999, 34:377-383) is also intriguing. While the technique is not yet sensitive enough for detecting endogenous peptides, it has been demonstrated that addition of exogenous peptide to the dialysis probe allows observation of in vivo processing of the peptide.

Recent advances in ‘top-down’ protein sequencing by electron capture dissociation (ECD) in a Fourier-transform ion cyclotron resonance mass spectrometer (FT-ICRMS) (McLafferty, F. W. International Journal of Mass Spectrometry, 2001, 212:81-87) suggest that larger peptides will soon be accessible by CLC-MS². However, this poses even greater computational problems for peptide sequencing and protein precursor identification by database searching. There are no elegant computational solutions at this time; although, probability-based scoring systems such as Mascot significantly improve confidence in the results (MacCoss, M. J. et al. Analytical Chemistry, 2002, 74:5593-5599; Perkins, D. N. et al. Electrophoresis, 1999, 20:3551-3567; Loh, S. Y. and McLafferty, F. W. Analytical Chemistry, 1991, 63:546-550; Stauffer, D. B. and McLafferty, F. W. Organic Mass Spectrometry, 1986, 21:313-315; Atwater, B. L. et al. Analytical Chemistry, 1985, 57:899-903; Stauffer, D. B. et al. Analytical Chemistry, 1985, 57:1056-1060; Pesyna, G. M. et al. Analytical Chemistry, 1976, 48:1362-1368; Mclaffer, F. W. et al. Organic Mass Spectrometry, 1974, 9:690-702).

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. An isolated peptide selected from the group consisting of YPVEP (SEQ ID NO: 2), YPVEPEEE (SEQ ID NO: 3), SPQLEDEAKE (SEQ ID NO: 4), SPQLEDEAKELQ (SEQ ID NO: 5), VGRPEWWMDYQ (SEQ ID NO: 6), YSKEVPEME (SEQ ID NO: 8), RKGPGPGGPGGAGGARGGAGGGPSGD (SEQ ID NO: 9), KGPGPGGPGGAGGARGGAGGGP (SEQ ID NO: 10), KGPGPGGPGGAGGARGGAGGGPSGD (SEQ ID NO: 11), GPGPGGPGGAGGARGGAGGGP (SEQ ID NO: 12), GPGPGGPGGAGGARGGAGGGPS (SEQ ID NO: 13), GPGPGGPGGAGGARGGAGGGPSGD (SEQ ID NO: 14), GPGGPGGAGGARGGAGGGPSGD (SEQ ID NO: 15), ADTGTTDEFIEAGGDIR (SEQ ID NO: 16), DTGTTDEFIEAGGDIR (SEQ ID NO: 17), EFIEAGGDIR (SEQ ID NO: 18), SPVPDLVPG (SEQ ID NO: 19), SQLQEGPPEWK (SEQ ID NO: 20), LVQTQAATDSDKVDLSIAR (SEQ ID NO: 21), TTDSDKVDLSIA (SEQ ID NO: 22), TDSDKVDLSIAR (SEQ ID NO: 23), IAQDNEPEKPVAKSETKM (SEQ ID NO: 24), QDNEPEKPVADSETKM (SEQ ID NO: 25), DNEPEKPVADSETKM (SEQ ID NO: 26), EPEKPVADSETKM (SEQ ID NO: 27), AKAPAPAAPAAEPQAEAPVAS (SEQ ID NO: 28), AKAPAPAAPAAEPQAEAPVASSEQSVAVKE (SEQ ID NO: 29), SEQ ID NO: 31, SEQ ID NO: 32, SEQ ID NO: 33, SEQ ID NO: 34, SEQ ID NO: 35, SEQ ID NO: 36, and SEQ ID NO: 37, or fragments and variants of any of the foregoing.

2. An isolated nucleic acid sequence encoding a peptide selected from the group consisting of YPVEP (SEQ ID NO: 2), YPVEPEEE (SEQ ID NO: 3), SPQLEDEAKE (SEQ ID NO: 4), SPQLEDEAKELQ (SEQ ID NO: 5), VGRPEWWMDYQ (SEQ ID NO: 6), YSKEVPEME (SEQ ID NO: 8), RKGPGPGGPGGAGGARGGAGGGPSGD (SEQ ID NO: 9), KGPGPGGPGGAGGARGGAGGGP (SEQ ID NO: 10), KGPGPGGPGGAGGARGGAGGGPSGD (SEQ ID NO: 11), GPGPGGPGGAGGARGGAGGGP (SEQ ID NO: 12), GPGPGGPGGAGGARGGAGGGPS (SEQ ID NO: 13), GPGPGGPGGAGGARGGAGGGPSGD (SEQ ID NO: 14), GPGGPGGAGGARGGAGGGPSGD (SEQ ID NO: 15), ADTGTTDEFIEAGGDIR (SEQ ID NO: 16), DTGTTDEFIEAGGDIR (SEQ ID NO: 17), EFIEAGGDIR (SEQ ID NO: 18), SPVPDLVPG (SEQ ID NO: 19), SQLQEGPPEWK (SEQ ID NO: 20), LVQTQAATDSDKVDLSIAR (SEQ ID NO: 21), TTDSDKVDLSIA (SEQ ID NO: 22), TDSDKVDLSIAR (SEQ ID NO: 23), IAQDNEPEKPVAKSETKM (SEQ ID NO: 24), QDNEPEKPVADSETKM (SEQ ID NO: 25), DNEPEKPVADSETKM (SEQ ID NO: 26), EPEKPVADSETKM (SEQ ID NO: 27), AKAPAPAAPAAEPQAEAPVAS (SEQ ID NO: 28), AKAPAPAAPAAEPQAEAPVASSEQSVAVKE (SEQ ID NO: 29), SEQ ID NO: 31, SEQ ID NO: 32, SEQ ID NO: 33, SEQ ID NO: 34, SEQ ID NO: 35, SEQ ID NO: 36, and SEQ ID NO: 37, or fragments and variants of any of the foregoing.

3. A host cell transformed with a nucleic acid sequence, wherein said nucleic acid sequence encodes a peptide selected from the group consisting of YGGFM (SEQ ID NO: 1), YPVEP (SEQ ID NO: 2), YPVEPEEE (SEQ ID NO: 3), SPQLEDEAKE (SEQ ID NO: 4), SPQLEDEAKELQ (SEQ ID NO: 5), VGRPEWWMDYQ (SEQ ID NO: 6), YGGFL (SEQ ID NO: 7), YSKEVPEME (SEQ ID NO: 8), RKGPGPGGPGGAGGARGGAGGGPSGD (SEQ ID NO: 9), KGPGPGGPGGAGGARGGAGGGP (SEQ ID NO: 10), KGPGPGGPGGAGGARGGAGGGPSGD (SEQ ID NO: 11), GPGPGGPGGAGGARGGAGGGP (SEQ ID NO: 12), GPGPGGPGGAGGARGGAGGGPS (SEQ ID NO: 13), GPGPGGPGGAGGARGGAGGGPSGD (SEQ ID NO: 14), GPGGPGGAGGARGGAGGGPSGD (SEQ ID NO: 15), ADTGTTDEFIEAGGDIR (SEQ ID NO: 16), DTGTTDEFIEAGGDIR (SEQ ID NO: 17), EFIEAGGDIR (SEQ ID NO: 18), SPVPDLVPG (SEQ ID NO: 19), SQLQEGPPEWK (SEQ ID NO: 20), LVQTQAATDSDKVDLSIAR (SEQ ID NO: 21), TTDSDKVDLSIA (SEQ ID NO: 22), TDSDKVDLSIAR (SEQ ID NO: 23), IAQDNEPEKPVAKSETKM (SEQ ID NO: 24), QDNEPEKPVADSETKM (SEQ ID NO: 25), DNEPEKPVADSETKM (SEQ ID NO: 26), EPEKPVADSETKM (SEQ ID NO: 27), AKAPAPAAPAAEPQAEAPVAS (SEQ ID NO: 28), AKAPAPAAPAAEPQAEAPVASSEQSVAVKE (SEQ ID NO: 29), SEQ ID NO: 31, SEQ ID NO: 32, SEQ ID NO: 33, SEQ ID NO: 34, SEQ ID NO: 35, SEQ ID NO: 36, and SEQ ID NO: 37, or fragments and variants of any of the foregoing.

4. The host cell of claim 3, wherein said nucleic acid sequence further comprises a promoter sequence operably linked to said nucleic acid sequence, wherein said promoter sequence drives expression of said nucleic acid sequence within said host cell.

5. The host cell of claim 3, wherein said host cell is prokaryotic.

6. The host cell of claim 3, wherein said host cell is eukaryotic.

7. A recombinant construct a nucleic acid sequence, wherein said nucleic acid sequence encodes a peptide selected from the group consisting of YGGFM (SEQ ID NO: 1), YPVEP (SEQ ID NO: 2), YPVEPEEE (SEQ ID NO: 3), SPQLEDEAKE (SEQ ID NO: 4), SPQLEDEAKELQ (SEQ ID NO: 5), VGRPEWWMDYQ (SEQ ID NO: 6), YGGFL (SEQ ID NO: 7), YSKEVPEME (SEQ ID NO: 8), RKGPGPGGPGGAGGARGGAGGGPSGD (SEQ ID NO: 9), KGPGPGGPGGAGGARGGAGGGP (SEQ ID NO: 10), KGPGPGGPGGAGGARGGAGGGPSGD (SEQ ID NO: 11), GPGPGGPGGAGGARGGAGGGP (SEQ ID NO: 12), GPGPGGPGGAGGARGGAGGGPS (SEQ ID NO: 13), GPGPGGPGGAGGARGGAGGGPSGD (SEQ ID NO: 14), GPGGPGGAGGARGGAGGGPSGD (SEQ ID NO: 15), ADTGTTDEFIEAGGDIR (SEQ ID NO: 16), DTGTTDEFIEAGGDIR (SEQ ID NO: 17), EFIEAGGDIR (SEQ ID NO: 18), SPVPDLVPG (SEQ ID NO: 19), SQLQEGPPEWK (SEQ ID NO: 20), LVQTQAATDSDKVDLSIAR (SEQ ID NO: 21), TTDSDKVDLSIA (SEQ ID NO: 22), TDSDKVDLSIAR (SEQ ID NO: 23), IAQDNEPEKPVAKSETKM (SEQ ID NO: 24), QDNEPEKPVADSETKM (SEQ ID NO: 25), DNEPEKPVADSETKM (SEQ ID NO: 26), EPEKPVADSETKM (SEQ ID NO: 27), AKAPAPAAPAAEPQAEAPVAS (SEQ ID NO: 28), AKAPAPAAPAAEPQAEAPVASSEQSVAVKE (SEQ ID NO: 29), SEQ ID NO: 31, SEQ ID NO: 32, SEQ ID NO: 33, SEQ ID NO: 34, SEQ ID NO: 35, SEQ ID NO: 36, and SEQ ID NO: 37, or fragments and variants of any of the foregoing.

8. The recombinant construct of claim 7, wherein said recombinant construct is a viral vector.

9. The recombinant construct of claim 7, wherein said recombinant construct is a non-viral vector.

10. The recombinant construct of claim 7, wherein said recombinant construct further comprises a promoter sequence operably linked to said nucleotide sequence, wherein said promoter sequence drives expression of said nucleotide sequence.

11. A method for increasing endogenous levels of gamma-aminobutyric acid (GABA) or aspartate within a subject, wherein said method comprises administering a peptide or a nucleotide sequence encoding said peptide to the subject, wherein said peptide is selected from the group consisting of YGGFM (SEQ ID NO: 1), YPVEP (SEQ ID NO: 2), YPVEPEEE (SEQ ID NO: 3), SPQLEDEAKE (SEQ ID NO: 4), SPQLEDEAKELQ (SEQ ID NO: 5), VGRPEWWMDYQ (SEQ ID NO: 6), YGGFL (SEQ ID NO: 7), YSKEVPEME (SEQ ID NO: 8), RKGPGPGGPGGAGGARGGAGGGPSGD (SEQ ID NO: 9), KGPGPGGPGGAGGARGGAGGGP (SEQ ID NO: 10), KGPGPGGPGGAGGARGGAGGGPSGD (SEQ ID NO: 1 1), GPGPGGPGGAGGARGGAGGGP (SEQ ID NO: 12), GPGPGGPGGAGGARGGAGGGPS (SEQ ID NO: 13), GPGPGGPGGAGGARGGAGGGPSGD (SEQ ID NO: 14), GPGGPGGAGGARGGAGGGPSGD (SEQ ID NO: 15), ADTGTTDEFIEAGGDIR (SEQ ID NO: 16), DTGTTDEFIEAGGDIR (SEQ ID NO: 17), EFIEAGGDIR (SEQ ID NO: 18), SPVPDLVPG (SEQ ID NO: 19), SQLQEGPPEWK (SEQ ID NO: 20), LVQTQAATDSDKVDLSIAR (SEQ ID NO: 21), TTDSDKVDLSIA (SEQ ID NO: 22), TDSDKVDLSIAR (SEQ ID NO: 23), IAQDNEPEKPVAKSETKM (SEQ ID NO: 24), QDNEPEKPVADSETKM (SEQ ID NO: 25), DNEPEKPVADSETKM (SEQ ID NO: 26), EPEKPVADSETKM (SEQ ID NO: 27), AKAPAPAAPAAEPQAEAPVAS (SEQ ID NO: 28), AKAPAPAAPAAEPQAEAPVASSEQSVAVKE (SEQ ID NO: 29), SEQ ID NO: 30, SEQ ID NO: 31, SEQ ID NO: 32, SEQ ID NO: 33, SEQ ID NO: 34, SEQ ID NO: 35, SEQ ID NO: 36, and SEQ ID NO: 37, or biologically active fragments and variants of any of the foregoing.

12. The method of claim 11, wherein the subject is a mammal.

13. The method of claim 11, wherein the subject is human.

14. A method for increasing endogenous levels of gamma-aminobutyric acid (GABA) or aspartate within a subject, wherein said method comprises administering preproenkephalin A (PEA), or a biologically active fragment thereof to the subject.

15. The method of claim 14, wherein said biologically active fragments are selected from the group consisting of YGGFM (SEQ ID NO: 1), YPVEP (SEQ ID NO: 2), YPVEPEEE (SEQ ID NO: 3), SPQLEDEAKE (SEQ ID NO: 4), SPQLEDEAKELQ (SEQ ID NO: 5), VGRPEWWMDYQ (SEQ ID NO: 6), YGGFL (SEQ ID NO: 7), and YSKEVPEME (SEQ ID NO: 8).

16. The method of claim 14, wherein the subject is a mammal.

17. The method of claim 14, wherein the subject is human.

18. A method for increasing levels of gamma-aminobutyric acid (GABA) or aspartate in vitro, wherein said method comprises administering a peptide or a nucleotide sequence encoding said peptide to a plurality of cells in vitro, wherein said peptide is selected from the group consisting of YGGFM (SEQ ID NO: 1), YPVEP (SEQ ID NO: 2), YPVEPEEE (SEQ ID NO: 3), SPQLEDEAKE (SEQ ID NO: 4), SPQLEDEAKELQ (SEQ ID NO: 5), VGRPEWWMDYQ (SEQ ID NO: 6), YGGFL (SEQ ID NO: 7), YSKEVPEME (SEQ ID NO: 8), RKGPGPGGPGGAGGARGGAGGGPSGD (SEQ ID NO: 9), KGPGPGGPGGAGGARGGAGGGP (SEQ ID NO: 10), KGPGPGGPGGAGGARGGAGGGPSGD (SEQ ID NO: 11), GPGPGGPGGAGGARGGAGGGP (SEQ ID NO: 12), GPGPGGPGGAGGARGGAGGGPS (SEQ ID NO: 13), GPGPGGPGGAGGARGGAGGGPSGD (SEQ ID NO: 14), GPGGPGGAGGARGGAGGGPSGD (SEQ ID NO: 15), ADTGTTDEFIEAGGDIR (SEQ ID NO: 16), DTGTTDEFIEAGGDIR (SEQ ID NO: 17), EFIEAGGDIR (SEQ ID NO: 18), SPVPDLVPG (SEQ ID NO: 19), SQLQEGPPEWK (SEQ ID NO: 20), LVQTQAATDSDKVDLSLIR (SEQ ID NO: 21), TTDSDKVDLSIA (SEQ ID NO: 22), TDSDKVDLSIAR (SEQ ID NO: 23), IAQDNEPEKPVAKSETKM (SEQ ID NO: 24), QDNEPEKPVADSETKM (SEQ ID NO: 25), DNEPEKPVADSETKM (SEQ ID NO: 26), EPEKPVADSETKM (SEQ ID NO: 27), AKAPAPAAPAAEPQAEAPVAS (SEQ ID NO: 28), AKAPAPAAPAAEPQAEAPVASSEQSVAVKE (SEQ ID NO: 29), SEQ ID NO: 30, SEQ ID NO: 31, SEQ ID NO: 32, SEQ ID NO: 33, SEQ ID NO: 34, SEQ ID NO: 35, SEQ ID NO: 36, and SEQ ID NO: 37, or biologically active fragments and variants of any of the foregoing.

19. The method of claim 19, wherein said peptide is selected from the group consisting of YGGFM (SEQ ID NO: 1), YPVEP (SEQ ID NO: 2), YPVEPEEE (SEQ ID NO: 3), SPQLEDEAKE (SEQ ID NO: 4), SPQLEDEAKELQ (SEQ ID NO: 5), VGRPEWWMDYQ (SEQ ID NO: 6), YGGFL (SEQ ID NO: 7), and YSKEVPEME (SEQ ID NO: 8).

20. An apparatus for separation and characterization of a biological sample, said apparatus comprising:
a) a means for collecting a biological sample;
b) a means for chromatographic separation in fluid communication with said collecting means, wherein said chromatographic separation means comprises a column with an inlet end and an outlet end, an inner diameter, and a length;
c) a means for detecting the biological sample separated by said chromatographic separation means;
d) a means for removeably positioning the outlet end of said column adjacent to said detecting means; and
e) a multi-pressure pumping system for transporting said biological sample from said sampling means to and through said column.

21. The apparatus of claim 20, wherein said collecting means is selected from a group consisting of a microdialysis probe and a sample port.

22. The apparatus of claim 20, wherein said collecting means is a microdialysis probe, wherein said microdialysis probe comprises inlet tubing and an outlet capillary, wherein said outlet capillary comprises fused-silica with an inner diameter and an outlet diameter.

23. The apparatus of claim 22, wherein said inner diameter of said outlet capillary is about 25 μm and said outlet capillary outer diameter is about 360 μm.

24. The apparatus of claim 22, further comprising said microdialysis probe having an active length of about 4 mm.

25. The apparatus of claim 20, wherein said column is a capillary comprising fused-silica.

26. The apparatus of claim 20, wherein said column is within the range of about 2 cm to about 20 cm in length.

27. The apparatus of claim 20, wherein said column is about 10 cm in length.

28. The apparatus of claim 20, wherein said column is about 2 cm in length.

29. The apparatus of claim 20, wherein said column's inner diameter is within the range of about 20 μm to about 30 μm.

30. The apparatus of claim 20, wherein said column further comprises particles packed within the inner diameter of said column, a frit contained within the inner diameter of said column located downstream of said particles, and an electrospray emitter at the outlet end of said column.

31. The apparatus of claim 30, wherein said particles are reversed-phase particles, wherein said reversed-phase particles have a diameter.

32. The apparatus of claim 30, wherein said diameter of said reversed-phase particles is about 5 μm.

33. The apparatus of claim 30, wherein said particles are packed within a 2 cm segment of said column.

34. The apparatus of claim 30, wherein said particles are adjacent to said frit.

35. The apparatus of claim 30, wherein said frit is macroporous.

36. The apparatus of claim 30, wherein said frit comprises a mixture of glycidyl methacrylate and trimethylolpropane trimethacrylate, and wherein said mixture is photopolymerized.

37. The apparatus of claim 30, wherein said frit is located about 3 cm or less from said electrospray emitter.

38. The apparatus of claim 30, wherein said frit is about 1 cm from said electrospray emitter.

39. The apparatus of claim 30, wherein said inner and outer diameters of said electrospray emitter narrow and terminate into a spray orifice having an inner diameter.

40. The apparatus of claim 39, wherein said inner diameter of said spray orifice is within the range of about 2 μm to about 5 μm.

41. The apparatus of claim 39, wherein said inner diameter of said electrospray emitter at said spray orifice is 3 μm.

42. The apparatus of claim 39, wherein said spray orifice is etched into sharp edges.

43. The apparatus of claim 20, wherein said detection means is a mass spectrometer.

44. The apparatus of claim 20, wherein said detection means is a quadruple ion trap spectrometer.

45. The apparatus of claim 20, wherein said positioning means comprises an actuating apparatus that provides sufficient force to align said outlet end of said column with said detecting means.

46. The apparatus of claim 20, wherein said positioning means comprises a servomotor-driven translation stage and motion controller and driver.

47. The apparatus of claim 20, wherein said positioning means comprises an X-Y-Z positioner.

48. The apparatus of claim 20, wherein said positioning means is located within the range of about 0.1 to about 5.0 mm from said detecting means when said positioning means and said detecting means are adjacent.

49. The apparatus of claim 20, wherein said positioning means is located about 0.5 mm from said detecting means when said positioning means and said detecting means are adjacent.

50. The apparatus of claim 20, wherein said pumping system comprises a preconcentrating pump and a plurality of gradient elution pumps with outlet streams, wherein said outlet streams of said plurality of gradient elution pumps are joined into one flow stream.

51. The apparatus of claim 50, wherein said preconcentrating pump is capable of operating at a flow rate limited by the pressure of said column.

52. The apparatus of claim 50, wherein said preconcentrating pump is capable of operating at a flowrate of about 350 nL/min to about 400 nL/min at a pressure of about 3200 psi.

53. The apparatus of claim 50, wherein said preconcentrating pump is capable of operating at a flowrate of about 370 nL/min at a pressure of about 3200 psi.

54. The apparatus of claim 50, wherein said plurality of gradient elution pumps comprises a first pump for pumping an organic phase and a second pump for pumping a solvent.

55. The apparatus of claim 50, wherein said gradient elution pumps are capable of operating at a flowrate sufficient to maintain stable chromatographic separating means.

56. The apparatus of claim 50, wherein said gradient elution pumps are capable of operating at a combined flowrate within the range of about 1 nL/min to about 5,000 nL/min at a pressure within the range of about 100 psi to about 150 psi.

57. The apparatus of claim 50, wherein said apparatus further comprises a tee comprising a first outlet and a second outlet, wherein said first outlet is connected to said chromatographic separation means and said second outlet is connected to a first splitter.

58. The apparatus of claim 50, wherein said gradient elution pumps operate at a combined flowrate of about 4 nL/min at a pressure of about 100 psi.

59. The apparatus of claim 50, further comprising a plurality of valves.

60. The apparatus of claim 59, wherein said plurality of valves comprises a first valve that actuates to select said preconcentrating pump or said gradient elution pumps and a second valve that actuates to select the biological sample for transfer onto the separation means or to select the biological sample for transfer to waste.

61. The apparatus of claim 60, wherein said first and second valves are six-port valves.

62. The apparatus of claim 61, wherein said first valve and said second valve are connected with at least one of the group selected from a capillary, a tube, a pipe, and a duct.

63. The apparatus of claim 59, further comprising a tee upstream of said chromatographic separating means, wherein said tee comprises a first outlet and a second outlet.

64. The apparatus of claim 63, wherein said first outlet comprises a capillary aligned with said chromatographic separating means.

65. The apparatus of claim 63, wherein said second outlet comprises a capillary and a HV union, wherein said capillary has a termination point that is selected from the group consisting of a shut-off valve and a second splitter.

66. The apparatus of claim 65, wherein said HV union is connected to a voltage source.

67. The apparatus of claim 65, wherein said second splitter is a capillary terminating at a waste stream.

68. The apparatus of claim 63, further comprising means for automating control of said collecting means, said chromatographic separating means, said detecting means, said positioning means and said multi-pressure pumping system.

69. The apparatus of claim 68, wherein said automating means comprises a computer process control program.

70. The apparatus of claim 68, further comprising a means for removing contaminants from said outlet end of said column.

71. The apparatus of claim 70, wherein said removing means comprises passing a pulse of nitrogen gas past said outlet end of said column.

72. A method for analyzing a biological sample, said method comprising:
a) collecting the biological sample from an organism;
b) loading the biological sample onto a separation column integrated with an electrospray emitter;
c) separating the biological sample into components;
d) preparing the separation column for transfer of components to a means for detecting the biological sample, which is separated into components;
e) detecting the biological sample, which is separated into components; and
f) actuating a plurality of valves to control said collecting, said loading, said separating, said preparing, and said detecting steps.

73. The method of claim 72, wherein the biological sample is collected at basal conditions.

74. The method of claim 72, wherein the biological sample is collected at stimulated conditions.

75. The method of claim 74, wherein said stimulated conditions are initiated by the infusion of a potassium ion rich solution to the organism.

76. The method of claim 72, wherein the plurality of valves are six-port valves, wherein said actuating comprises a first position and a second position.

77. The method of claim 76, wherein the six-port valves comprise a first valve with first and second positions, wherein the first position selects a preconcentrating pump and the second position selects gradient elution pumps, and a second valve with first and second positions, wherein the first position transfers the collected biological sample to the separation column and the second position transfers the collected biological sample to waste.

78. The method of claim 72, wherein said collecting step comprises obtaining the biological sample and loading the biological sample onto a sample loop upstream of the column.

79. The method of claim 78, wherein said obtaining step is selected from at least one of the group consisting of manually withdrawing the biological sample from an organism and automatically absorbing the biological sample using a microdialysis probe inserted into an anatomical area of an organism.

80. The method of claim 78, wherein said loading step is selected from at least one of the group consisting of manually injecting the biological sample through a sample and port into the sample loop and automatically transporting the biological sample from the microdialysis probe to the sample loop.

81. The method of claim 72, wherein said loading step comprises:
a) transferring the biological sample and an weak mobile phase to the separation column;
b) preconcentrating the biological sample in the weak mobile phase; and
c) desalting the separation column with the weak mobile phase.

82. The method of claim 81, wherein said transferring and said preconcentrating steps comprises actuating the first valve to the first position and actuating the second valve to the first position.

83. The method of claim 81, wherein said desalting step comprises transferring the weak mobile phase to the column and transferring the remainder of the biological sample to waste.

84. The method of claim 81, wherein said desalting step comprises removing weakly bound molecules from the column.

85. The method of claim 81, wherein said desalting step comprises actuating the first valve to the first position and the second valve to the second position.

86. The method of claim 81, wherein the weak mobile phase is 1% acetic acid in water.

87. The method of claim 81, wherein said separating step comprises eluting an strong mobile phase with an organic content through the separation column as a gradient, wherein the organic content of the strong mobile phase is increased at a rate sufficiently slow to elute strongly bound molecules from the column.

88. The method of claim 81, wherein said eluting step occurs at a flowrate within the range of 10 nL/min to about 4 μL/min.

89. The method of claim 87, wherein the strong mobile phase is 1% acetic acid in methanol.

90. The method of claim 72, wherein said separating step comprises actuating the first valve to the second position and actuating the second valve to the second position.

91. The method of claim 72, wherein said preparing step comprises:
a) removing any contaminants from the outlet of the separation column;
b) positioning the separation column adjacent to a means for detecting; and
c) applying a voltage to a HV union to initiate electrospray to detection means.

92. The method of claim 91, wherein said removing step comprises passing a pulse of nitrogen gas across the outlet of the separation column.

93. The method of claim 91, wherein said positioning step is actuating an apparatus with sufficient force to align said column with detecting means.

94. The method of claim 91, wherein said removing and positioning steps occur simultaneously.

95. The method of claim 91, wherein said removing step occurs before said positioning step.

96. The method of claim 91, wherein said removing, said positioning, and said applying steps comprise transferring an strong mobile phase to the column and transferring the biological sample to waste.

97. The method of claim 91, wherein said removing, said positioning, and said applying steps comprise actuating the first valve to the second position and actuating the second valve to the second position.

98. The method of claim 72, wherein said loading step occurs at a flowrate of about 350 nL/min to about 400 nL/min.

99. The method of claim 72, wherein said loading step occurs at a flowrate of about 370 nL/min.

100. The method of claim 72, further comprising simultaneously infusing at least one biologically active agent.

101. A method for analyzing a biological sample, said method comprising:
a) collecting a biological sample from an organism;
b) pumping, at high pressure and high flowrate, the biological sample and an weak mobile phase to a means for separating;
c) pumping, at low pressure and low flowrate, the biological sample and an strong mobile phase through the separating means;
d) separating the biological sample into components; and
e) characterizing the biological sample that is separated.

102. The method of claim 101, wherein said pumping at high pressure and high flowrate comprises pumping at a pressure of about 3200 psi within the range of about 350 nL/min to about 400 nL/min.

103. The method of claim 101, wherein said pumping at high pressure and high flowrate comprises pumping at a pressure of about 3200 psi at about 370 nL/min.

104. The method of claim 101, wherein said pumping at low pressure and low flowrate comprises pumping at about 150 psi within the range of about 1 nL/min to about 5,000 nL/min.

105. The method of claim 101, wherein said pumping at low pressure and low flowrate comprises pumping at about 150 psi at about 4 μL/min at the gradient pump and about 20 nL/min at the separating means.

106. A method for tuning a detection apparatus comprising:
a) collecting a standard for a biological sample;
b) infusing a tuning solution into a means for separating the standard, wherein the separating means is in fluid communication with an electrospray emitter;
c) eluting the tuning solution and the standard through the separating means;
d) removing any contaminant present on the electrospray emitter;
e) aligning the separating means with a means to detect components in the standard f) applying voltage to a liquid junction to initiate electrospray of components in the standard, wherein the electrospray transports components of the standard to the detection means; and
g) tuning the detection means.

107. The method of claim 106, wherein the standard is a 18 nM solution of neurotensin 1-11.

108. The method of claim 107, wherein the 18 nM solution of neurotensin is dissolved in a 50% methanol and 1% acetic acid solution.

109. The method of claim 106, wherein the electrospray emitter has an outlet, and wherein said removing comprises applying a pulse of gas across the outlet of the electrospray emitter.

110. The method of claim 106, further comprising:
h) splitting the tuning solution into an eluting stream and a waste stream.
